(19) United States
(12) Patent Application Publication
(10) Pub. No.: US 2023/0348665 A1
(43) Pub. Date: Nov. 2, 2023

(54) CHELATION CROSSLINKED POLYMERS, METHODS OF MAKING SAME, AND USES THEREOF

(71) Applicant: CORNELL UNIVERSITY, Ithaca, NY (US)

(72) Inventors: Yadong Wang, Ithaca, NY (US); Ying Chen, Ithaca, NY (US)

(21) Appl. No.: 18/006,476

(22) PCT Filed: Aug. 2, 2021
(86) PCT No.: PCT/US2021/044230

§ 371 Date: Jan. 23, 2023

Related U.S. Application Data
(60) Provisional application No. 63/060,057, filed on Aug. 1, 2020.

(51) Int. Cl. C08G 63/685; C08L 67/03; C08K 5/00

(52) U.S. Cl. CPC C08G 63/6856; C08L 67/03; C08K 5/0091; C08L 2203/02; C08L 2203/12; C08L 2203/14; C08L 2203/16; C08L 2201/06; C08L 2312/00

(57) ABSTRACT

The chelation crosslinked polymers include a polymer backbone having one or more chelation crosslinking group(s) at least partially bonded to one or more cation(s). The chelation crosslinking groups may be within a polymer backbone and/or pendant from the polymer backbone. The chelation crosslinked polymers may be chelation crosslinked polyesters. The chelation crosslinked polymers can be used in tissue engineering applications to form tissue grafts and scaffolds.

### CROSS-REFERENCE TO RELATED APPLICATIONS

This application claims the benefit of U.S. Provisional application No. 63/060,057 filed Aug. 1, 2020, the contents of the above-identified application are hereby fully incorporated herein by reference in their entirety.

### BACKGROUND OF THE DISCLOSURE

An elastomer is typically a polymer with a glass transition temperature (T g) lower than room temperature and with low plastic deformability. Elastic recoil makes an elastomer important for maintaining functions of natural tissues and man-made structures. Covalent bonds link random coiled polymers into elastomers such as elastin, resilin, silicone and vulcanized rubber. Weak bonds perform the same task in polyurethanes, polyamide and polyvinyl chloride. Each of these elastomers has its own chemistry that dictates a specific bond to crosslink into a network. Thus, the design must be tailored to each polymer and each resultant polymer will have a specific set of properties. This makes the elastomer design a laborious process and limits the versatility and range of properties of the resultant material.

### SUMMARY OF THE DISCLOSURE

In an aspect, the present disclosure provides a chelation crosslinked polymer. In various examples, a chelation crosslinked polymer (e.g., a chelation crosslinked polyester or the like) comprises a polymer backbone (e.g., a polyester backbone or the like) containing one or more repeat group(s) (e.g., ester group(s) or the like). In various examples, a chelation crosslinked polyester comprises a polyester backbone containing one or more ester group(s). In various examples, a polymer backbone (e.g., a polyester backbone or the like) comprises one or more chelation crosslinking group(s) within and/or pendant from the polymer backbone (e.g., the polyester backbone or the like). In various examples, a polyester backbone comprises one or more chelation crosslinking group(s) within and/or pendant from the polyester backbone. In various examples, a chelation crosslinked polymer (e.g., a chelation crosslinked polyester or the like) further comprises one or more cation(s). In various examples, a chelation crosslinked polyester further comprises one or more cation(s). In various examples, at least a portion of cation(s) is/are bonded to at least a portion of chelation crosslinking group(s) via one or more chelation crosslinking bond(s). In various examples, chelation crosslinking bond(s) crosslink a polymer backbone (e.g., a polyester backbone or the like). In various examples, chelation crosslinking bond(s) crosslink a polyester backbone. In various examples, one or more (or all) chelation crosslinking group(s) is/are pendant from a polymer backbone (e.g., a polyester backbone or the like). In various examples, one or more (or all) chelation crosslinking group(s) is/are pendant from a polyester backbone. A chelation crosslinked polymer (e.g., a chelation crosslinked polyester or the like) can comprise various types of chelation crosslinking bond(s) between chelation crosslinking group(s) and cation(s). In various examples, at least a portion of (or all) cation(s) is/are bonded to at least a portion of chelation crosslinking group(s) via bonds chosen from ionic bonds, coordinate covalent bonds, and the like, and combinations thereof. In various examples, crosslinking is reversible.

In various examples, a chelation crosslinked polymer (e.g., a chelation crosslinked polyester or the like) comprises a copolymer backbone (e.g., a polyester copolymer backbone or the like). In various examples, a chelation crosslinked polyester comprises a polyester copolymer backbone. A copolymer backbone (e.g., a polyester copolymer backbone) can comprise various structures. In various examples, a copolymer backbone (e.g., a polyester copolymer backbone) is a block copolymer backbone (e.g., a polyester block copolymer backbone) comprising one or more other block(s) (other block(s) are block(s) other than polymer backbone blocks(s) (e.g., a polyester backbone block(s) or the like)) chosen from one or more other hydrophilic block(s), one or more other hydrophobic block(s), and the like, and combinations thereof.

A chelation crosslinked polymer (e.g., a chelation crosslinked polyester or the like) can comprise various chelation crosslinking groups. In various examples, chelation crosslinking group(s) is/are chosen from polydentate chelation crosslinking groups, and the like, and combinations thereof. A chelation crosslinked polymer (e.g., a chelation crosslinked polyester or the like) can comprise various cation types and charges. In various examples, cation(s) is/are chosen from Group(II) cations, transition metals, and the like, and combinations thereof.

In an aspect, the disclosure provides compositions. In various examples, a composition comprises one or more chelation crosslinked polymer(s) (e.g., chelation crosslinked polyester(s) or the like) of the present disclosure. In various examples, a composition comprises one or more chelation crosslinked polyester(s). The compositions can be used in various applications. In various examples, a composition is a biomedical composition, a pharmaceutical composition, a chewing gum base, a sealant, or the like. In various examples, a composition is a fiber, a film, a monolith, a tube, a foam, or the like. In an aspect, the disclosure provides fibers. In various examples, a fiber comprises one or more chelation crosslinked polymer(s) (e.g., chelation crosslinked polyester(s) or the like) of the present disclosure. In various examples, a fiber comprises a blend of chelation crosslinked polymer(s) (e.g., chelation crosslinked polyester(s) or the like) and one or more other polymer(s) (other polymer(s) are not chelation crosslinked polymer(s) (e.g., chelation crosslinked polyester(s) or the like)) and/or one or more other polymeric material(s) (other polymeric material(s) do not comprise chelation crosslinked polymer(s) (e.g., chelation crosslinked polyester(s) or the like)).

In an aspect, the disclosure provides materials. In various examples, a material comprises a plurality of fibers of the present disclosure. A material can comprise various forms. In various examples, a material is a fabric or the like. In various examples, a fabric is a weave or braid of fibers or the like. In various examples, a material comprises one or more fibers having chelation crosslinked polymer(s) (e.g., chelation crosslinked polyester(s) or the like) and further comprises one or more other fiber(s) (other fiber(s) do(es) not comprise chelation crosslinked polymer(s) (e.g., chelation crosslinked polyester(s) or the like)). In various examples, a material comprises one or more fibers having chelation crosslinked polyester(s) and further comprises one or more other fiber(s) (other fiber(s) do(es) not comprise chelation crosslinked polyester(s)).

In an aspect, the disclosure provides tissue grafts. In various examples, a tissue graft comprises one or more chelation crosslinked polymer(s) (e.g., chelation crosslinked polyester(s) or the like) of the present disclosure. In various examples, a tissue graft comprises one or more chelation crosslinked polyester(s) of the present disclosure. A tissue graft can comprise various forms. In various examples, a tissue graft is a soft tissue graft or the like. In various examples, a soft tissue graft is a vascular graft or the like. In various examples, a vascular graft is an arterial graft or the like. In various examples, an arterial graft comprises a lumen diameter of 6 mm or less.

In an aspect, the disclosure provides articles of manufacture. In various examples, an article of manufacture comprises one or more chelation crosslinked polymer(s) (e.g., chelation crosslinked polyester(s) or the like) of the present disclosure. In various examples, an article of manufacture comprises one or more chelation crosslinked polyester(s) of the present disclosure. An article of manufacture can comprise various forms. In various examples, an article of manufacture is chosen from consumer goods, tires, gloves, gaskets, washers, toys, chewing gum, hoses, and balloons, and the like, and combinations thereof.

### BRIEF DESCRIPTION OF THE FIGURES

For a fuller understanding of the nature and objects of the disclosure, reference should be made to the following detailed description taken in conjunction with the accompanying figures herein.

FIGS. 1A-1C illustrate a material design and a polymer characterization. FIGS. 1A(i)-1A(iii): Synthesis of a 2-[[(2-Hydroxyphenyl)methylene]amino]-1,3-propanediol (HPA) monomer (FIG. 1A(i)), a poly(Propanediol-HPA-Sebacate) (PAS) (FIG. 1A(ii)) polymer, and a M-PAS (FIG. 1A(iii)) elastomer (x=ligand amounts, M=metal ions). FIGS. 1B(i)-1B(iii): Structural formula of a PAS polymer and an ¹H NMR (500 Hz) spectra of a 6-PAS polymer (FIG. 1B(i)), a 9-PAS polymer (FIG. 1B(ii)), and a 14-PAS polymer (FIG. 1B(iii)) in acetone-d₆. A ratio of integral of H_{b} and Hₐ determines an actual ligand amount. FIG. 1C: A gel permeation chromatography (GPC) of a 6-PAS polymer, a 9-PAS polymer and a 14-PAS polymer. Inset: a weight average molecular weight (Mw) and a polydispersity (PDI) of a 6-PAS polymer, a 9-PAS polymer, and a 14-PAS polymer. Data represent mean±SD, n=3.

FIGS. 2A-2B illustrates a gas chromatography-mass spectrometry (GC-MS) analysis of a synthesized HPA monomer. FIG. 2A: GC of HPA monomer. FIG. 2A Inset: a gross appearance of HPA monomer crystals and a structure of an HPA monomer. FIG. 2B: MS of HPA monomer.

FIGS. 3A-3B illustrate: (FIG. 3A) An ¹H NMR spectrum of an HPA monomer in an acetone-d₆ solvent. Inset: a structure of an HPA monomer; (FIG. 3B) Magnified peaks from a ¹H NMR spectrum of a HPA monomer. An integral area of Hₐ to Hₜ: A_{H}_{ᵢ}=1.0173, A_{H}_{ₑ}=1.000, A_{H}_{_{g}}=0.9719, A_{H}_{_{d}}=1.0557, A_{H}_{_{f+h}}=1.9006, A_{H}_{ₐ}=1.9105, A_{H}_{_{b}}=2.2751, A_{H}_{_{b}}=2.3617, A_{H}_{_{c}}=1.1736.

FIGS. 4A-4C illustrate: (FIG. 4A) a chelation reaction of an HPA monomer and CuCl₂ and a structure of a Cu(HPA)₂ chelate; (FIG. 4B) A UV-visible spectra of an HPA and a Cu(HPA)₂ chelate with a ligand/metal ratio of 2; (FIG. 4C) An ¹H NMR spectrum of an HPA monomer (black curve) and a Cu(HPA)₂ chelate (green curve) in acetone-d₆. Inset: images of an HPA and a Cu(HPA)₂ chelate solution. All peaks are broadened due to paramagnetic copper (II). Changes in chemical shifts are caused by the chelation between copper (II) and Schiff-base.

FIGS. 5A-5C illustrate: (FIG. 5A) a structure of a 6-PAS polymer; (FIG. 5B) An ¹H NMR spectrum of a 6-PAS polymer in acetone-d₆; (FIG. 5C) Magnified peaks from an ¹H NMR spectrum of a 6-PAS polymer. An integral area of Hₐ to Hₘ: A_{H}_{ₖ}=0.0022, A_{H}_{_{b}}=0.0620, A_{H}_{_{g}}=0.0150, A_{H}_{ᵢ}=0.0158, A_{H}_{ₕ₊ⱼ}=0.0346, Aₑ=0.0368, A_{H}_{ₗ}=0.8787, A_{H}_{_{f}}=0.0164, A_{H}_{ₑ}=0.0426, A_{H}_{_{d}}=0.9641, A_{H}_{ₘ}=0.3740, A_{H}_{ₐ}=1.0000, A_{H}_{_{c}}=2.1151.

FIG. 6 illustrates a typical GPC chromatogram for an intermediate, a polycondensation product of 1, 3-propanediol and sebacic acid with a molar ratio of 0.85:1, 0.80:1 or 0.75:1. Inset: weight average molecular weight (Mw) and polydispersity (PDI) of an intermediate product. Data represent mean±SD, N=3.

FIGS. 7A-7D illustrate: (FIG. 7A) DSC curves for a 6-PAS polymer; (FIG. 7B) a 9-PAS polymer; (FIG. 7C) 14-PAS polymer (FIG. 7C); (FIG. 7D) a summary of a glass transition temperature (T_{g}), melting temperature (Tₘ), a crystallization temperature (T_{c}), an enthalpy of melting (ΔHₘ) and an enthalpy of crystallization (ΔH_{c}) for a 6-PAS polymer, a 9-PAS polymer and a 14-PAS polymer.

FIGS. 8A-8B illustrate: (FIGS. 8A(i)-8A(iv)) a gross appearance of a 14-PAS polymer cured at 30 mTorr and 150° C. for 8 hours and then cooled for 30 min (min=minute(s)) to room temperature (FIGS. 8A(i)-8(ii)). A cured 14-PAS polymer becomes brittle solid after being cooled overnight (FIG. 8A(iii)) and can dissolve in acetone completely (FIG. 8A(iv)). (FIGS. 8B(i)-8B(ii)) ¹H NMR spectra of a 14-PAS polymer before (FIG. 8B(i)) and after (FIG. 8B(ii)) curing at 150° C. and 30 mTorr for 8 hours.

FIG. 9 illustrates a comparison of an FTIR spectra of a 9-PAS polymer and a 9-Fe-PAS elastomer.

FIGS. 10A-10D illustrate versatility, degradation and cytocompatibility of an M-PAS elastomer. (FIGS. 10A(i)-10A(iv)) A 9-Cu-PAS elastomer film (FIG. 10A(i)), a 9-Cu-PAS elastomer foam (FIG. 10A(ii)) and a 9-Cu-PAS elastomer porous tube (FIG. 10A(iii)). A typical M-PAS elastomer is highly elastic. The shape recovers rapidly after release of external force. SEM images of cross-sections of a 9-Cu-PAS elastomer porous tube (FIG. 10A(iv)). (FIG. 10B) Degradation of a 14-M-PAS elastomer in a basic solution. (FIGS. 10C(i)-10C(ii)) Photographs (FIG. 10C(i)) and degradation (FIG. 10C(ii)) of a 6-M-PAS elastomer film in an EDTA solution. (FIGS. 10D(i)-10D(ii)) Cell morphology, live/dead staining (FIG. 10D(i)) and metabolic activity (FIG. 10D(ii)) of HUVECs after 6 days' culture on a 14-Cu-PAS elastomer coating and a PLGA coating. Data represent mean±SD, n=3. Significant difference: *, p<0.05; **, p<0.01.

FIG. 11 illustrates representative three-dimensional reconstruction images of a 9-Cu-PAS elastomer porous tube after Micro-CT scanning.

FIGS. 12A-12D illustrate a control of mechanical properties of a M-PAS elastomer by ligand/metal ratio, ligand density and metal ion types. (FIGS. 12A(i)-12A(iii)) Photographs (FIG. 12A(i)) and UV-visible spectra (FIG. 12A(ii)) of a 9-Fe-PAS elastomer with different ligand/metal ratios. Arrows: peaks shift of a salicylaldimine side group. Stress-strain curves of a 9-Fe-PAS elastomer film with different metal/ligand ratios (FIG. 12A(iii)). (FIG. 12B) Stress-strain curves of a x-Co-PAS elastomer, x=6, 9 and 14. (FIG. 12C) Stress-strain curves of a 14-M-PAS elastomer film with different metal ions. (FIGS. 12D(i)-12D(vi)) Hysteresis tests of a 14-M-PAS elastomer film. M=Mg²⁺ (FIG. 12D(i)), Ca²⁺ (FIG. 12D(ii)), Fe³⁺ (FIG. 12D(iii)), Co²⁺ (FIG. 12D(iv)), Cu²⁺ (FIG. 12D(v)), and Zn²⁺ (FIG. 12D(vi)).

FIGS. 13A-13B illustrate: (FIGS. 13A(i)-13A(iv)) a comparison of strain at fracture % (FIG. 13A(i)), ultimate tensile strength (UTS, kPa) (FIG. 13A(ii)), Young's modulus (MPa) (FIG. 13A(iii)) and toughness (mJ) (FIG. 13A(iv)) of a 9-Fe-PAS elastomer film at different ligand/metal ratios. (FIG. 13B) Summary of mechanical test data for a 9-Fe-PAS elastomer film with different ligand/metal ratios. Data represent mean±SD, n=4.

FIGS. 14A-14B illustrate: (FIGS. 14A(i)-14A(iv)) a comparison of strain at fracture % (FIG. 14A(i)), ultimate tensile strength (UTS, kPa) (FIG. 14A(ii)), Young's modulus (MPa) (FIG. 14A(iii)) and toughness (mJ) (FIG. 14A(iv)) of a 6-, 9-, and 14-Co-PAS elastomer film. (FIG. 14B) Summary of mechanical test data for a 6-, 9-, and 14-Co-PAS elastomer film. Data represent mean±SD, n=4.

FIGS. 15A-15B illustrate: (FIGS. 15A(i)-15A(iv)) a comparison of strain at fracture % (FIG. 15A(i)), ultimate tensile strength (UTS, kPa) (FIG. 15A(ii)), Young's modulus (MPa) (FIG. 15A(iii)) and toughness (mJ) (FIG. 15A(iv)) of a 14-M-PAS elastomer film. (FIG. 15B) Summary of mechanical test data for a 14-M-PAS elastomer film. Data represent mean±SD, n=4.

FIGS. 16A-16B illustrate a hydrophilicity test of a M-PAS elastomer film. (FIG. 16A) contact angles of a 6-, 9-, and 14-Co-PAS elastomer film. (FIG. 16B) contact angles of a 14-M-PAS elastomer film. M=Mg²⁺, Ca²⁺, Fe³⁺, Co²⁺, Cu²⁺ and Zn²⁺, respectively. Data represent mean±SD, n=4.

FIGS. 17A-17B illustrate SEM images of cross-sections of a 14-Fe-PAS elastomer foam (FIG. 17A) and a PCL polymer foam (FIG. 17B) in low, middle and high magnifications.

FIG. 18 illustrates photographs of a 14-Fe-PAS elastomer foam and a PCL polymer foam after being implanted symmetrically in the dorsal of mice for 84 days.

FIGS. 19A-19B illustrate a subcutaneous implantation of 14-Fe-PAS elastomer foam in mice. (FIG. 19A) Gross appearance of a 14-Fe-PAS elastomer foam and a PCL polymer foam after being implanted under the dorsal skin of BALB-CJ mice for 4, 14, 28, 56 and 84 days. Unit of ruler: mm. (FIG. 19B) Photomicrographs of H&E staining for cross-sections of the implants after in vivo implantation. Slides are obtained by sectioning at the center of each implant. Scale bars for low magnification: 1.0 mm; high magnification: 200 μm.

FIGS. 20A-20B illustrate photomicrographs of H&E staining for the cross-sections of a 14-Fe-PAS elastomer foam (FIG. 20A) and a PCL polymer foam (FIG. 20B) after being implanted in the dorsal of mice for 4, 14, 28, 56 and 84 days. The slides are obtained by sectioning at the center, quarter and edge of each implant. Scale bars for low magnification: 1.0 mm; high magnification: 200 μm.

FIG. 21 illustrates photomicrographs of H&E staining in high magnification for a 14-Fe-PAS elastomer foam and a PCL polymer foam after being implanted in the dorsal of mice for 4, 14, 28, 56 and 84 days. Scale bar: 200 μm.

FIG. 22 illustrates a comparison of the degradation of 14-Fe-PAS elastomer foam and a PCL polymer foam soaked in a pH=12.63 solution for 4, 24 and 48 hours. Data represent mean±SD, n=4.

FIGS. 23A-23D illustrate a host response of implants in mice. (FIG. 23A) Immunofluorescence staining of CD68 positive macrophages merged with DAPI staining for cross-sections of the implants after in vivo implantation for 4, 14, 28, 56 and 84 days. Red signals show the presence of macrophages in the implants. Blue signals show cell nuclei. (FIG. 23B) MTS staining for cross-sections of the implants. Slides are obtained by sectioning at the center of each implant. Scale bars for low magnification: 1.0 mm; high magnification: 200 μm. (FIGS. 23C(i)-23C(iv)) Granulocytes (FIG. 23C(i)), macrophages (FIG. 23C(ii)), LC-PC inflammation (FIG. 23C(iii)) and CT thickness (FIG. 23C(iv)) in tissue surrounding implants. (FIGS. 23D(i)-23D(iv)) Granulocytes (FIG. 23D(i)), macrophages (FIG. 23D(ii)), LC-PC inflammation (FIG. 23D(iii)) and necrosis (FIG. 23D(iv)) within the implants. LC-PC=lymphoplasmacytic, CT=connective tissue. Data represent mean±SD, n=5. Significant difference: *, p<0.05.

FIGS. 24A-24B illustrate immunofluorescence staining of CD68 positive macrophages merged with DAPI staining for cross-sections of a 14-Fe-PAS elastomer foam (FIG. 24A) and a PCL polymer foam (FIG. 24B) after implantation in the dorsal of mice for 4, 14, 28, 56 and 84 days. Red signals show the presence of macrophages in implants. Blue signals show cell nuclei. Slides are obtained by sectioning at the center, quarter and edge of each implant. Scale bars for low magnification: 1.0 mm; for high magnification: 200 μm.

FIGS. 25A-25B illustrate MTS staining for cross-sections of a 14-Fe-PAS elastomer foam (FIG. 25A) and a PCL polymer foam (FIG. 25B) after being implanted in dorsal of mice for 14, 28, 56 and 84 days. The slides are obtained by sectioning at the center, quarter and edge of each implant. Scale bars for low magnification: 1.0 mm; high magnification: 200 μm.

FIGS. 26A-26B illustrate a comparison of host response of a 14-Fe-PAS elastomer foam and a PCL polymer foam after in vivo implantation for 4, 14, 28, 56 and 84 days: (FIGS. 26A(i)-26A(iii)) Fibroblasts (FIG. 26A(i)), MNGC (FIG. 26A(ii)) and fibrillar CT (FIG. 26A(iii)) in the tissue surrounding the implants. (FIGS. 26B(i)-26B(iv)) Fibroblasts (FIG. 26B(i)), MNGC (FIG. 26B(ii)), collagen (FIG. 26B(iii)) and capillaries (FIG. 26B(iv)) within the implants. CT=connective tissue, MNGC=multinucleated giant cells. Data represent mean±SD, n=5. Significant difference: *, p<0.05.

FIG. 27 illustrates accelerated H₂O₂ decomposition by a M-PAS elastomer (9-Cu-PAS and 9-Fe-PAS) versus an H₂O₂-only control from 0 to 24 hours (h). Significance of ANOVA of different reaction conditions at specific durations are labeled with an asterisk and a solid line (p<0.0001). Significance of comparisons between 9-Cu-PAS and 9-Fe-PAS elastomer and controls at specific durations are labeled with an asterisk and dotted lines (p<0.0005).

FIGS. 28A-C illustrate: (FIG. 28A) Macroscopic views of a 11-Zn-PAS graft upon implantation and 7 days post-operation. (FIG. 28B). Schematic of the sectioning positions for histological analysis. Slides were obtained by sectioning at proximal site (PS), left quarter (M1), center (M2), and right quarter (M3) of each implant. (FIG. 28C). Photomicroscopy images of H&E staining for cross-sections of the implants after being implanted for 3, 5, and 7 days.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range.

As used herein, unless otherwise stated, the term “group” refers to a chemical entity that is monovalent (i.e., comprises one terminus that can be covalently bonded to other chemical species), divalent, or polyvalent (i.e., comprises two or more termini that can be covalently bonded to other chemical species). The term “group” also includes radicals (e.g., monovalent and multivalent, such as, for example, divalent radicals, trivalent radicals or the like). Illustrative examples of groups include:

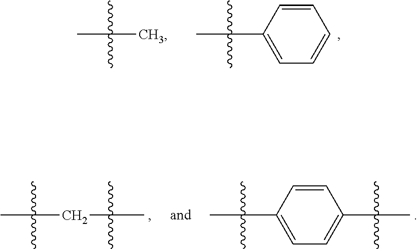

As used herein, unless otherwise stated, the terms “pendant” or “pendant group” or “side group” or “ligand” are used interchangeably and refer to a group attached to a polymer backbone. A pendant group may be directly attached to a polymer backbone or a linking group may connect a pendant group to a polymer backbone.

As used herein, the term “chelation crosslinking bonding” or “chelation crosslinking bond(s)” refers to individual cation(s) which is/are bonded (e.g., by a plurality of ionic bonds, coordination bonds, or the like, or a combination thereof) to two or more chelation crosslinking groups that results in crosslinking, e.g. inter-chain crosslinking, intra-chain crosslinking, or a combination thereof.

As used herein, unless otherwise stated, the term “room temperature” refers to a temperature range of 18° C. to 30° C. (64° F. to 86° F.), including all 1° C. (1° F.) values and ranges therebetween.

The present disclosure provides chelation crosslinked polymers. The present disclosure also provides methods of making chelation crosslinked polymers and uses of chelation crosslinked polymers.

The present disclosure addresses elastomer design problems using chelation bonds. This design is versatile in that one ligand may bind different kinds of metal ions. The resultant bonds (e.g., coordination bonds or the like) can have, for example, different strengths, leading to, for example, different mechanical properties and biodegradability.

The instant polymer design emphasizes various factors. For example, the type of bonds (e.g., coordination bonds or the like): When a metal-ligand interaction involves multiple bonds, it becomes a chelation bond. Chelation bonds may be used in order to have a wider range of bond strength and mechanical properties in the resultant polymers. In another example, the type of ligand: In various examples, the ligand is effectively half a salen ligand. This ligand provides two coordination bonds so that two and three of the pendant groups on the polymer chains create a tetradentate and hexadentate ligand respectively for the appropriate metal ions to nucleate the crosslinks. In yet another example, polymer backbone and metal ions: For example, in the case of biomedical applications, polymer backbones with degradable ester bonds and biologically relevant metal ions: Mg²⁺, Ca²⁺, Fe³⁺, Cu²⁺, Zn²⁺ Co²⁺, or the like, may be desirable. For example, a diacid monomer is sebacic acid because of its known biocompatibility and the diols may be a Schiff-base ligand (2-[[(2-hydroxyphenyl)methylene]amino]-1,3-propanediol, HPA) and 1, 3-propanediol. The former derives from serinol, offering good biocompatibility. 1, 3-propanediol is a common food additive, pharmaceutical excipient, and has demonstrated safety in vivo.

The identity of the metal ions, metal/ligand ratio, and ligand density may impact the mechanical properties of the resultant polymers. Using the Cu²⁺ or Fe³⁺ crosslinked polymers (e.g., elastomers or the like) as examples, culture of human umbilical vein endothelial cells and subcutaneous implantation in mice reveal their biocompatibility and biodegradability. The crosslinking mechanism of the present disclosure affords polymers with a wide range of mechanical properties and desirable biocompatibility.

Without intending to be bound by any particular theory, it is considered that the mechanical properties and degradability of the polymers (e.g., elastomers or the like) can be controlled by changing the crosslinking density, which in turn is determined by the percentage of the crosslinking groups of the polymers (e.g., salicylaldimine side groups on the backbone (such as, for example, 6%, 9% and 14%), which may be referred to as ligands. Higher ratio of crosslinking groups provides more chelation sites for metal ions, resulting in higher crosslinking density and typically, tougher mechanical properties of the resultant polymers (e.g., elastomers or the like). Additionally, it is considered that the mechanical properties and degradability of the polymers (e.g., elastomers or the like) can be controlled by selecting the metal ion(s) and the molar ratio(s) of metal ion(s) to crosslinking groups in the polymers. For example, a series of biologically relevant metal ions (e.g., Cu²⁺, Ca²⁺, Co²⁺, Mg²⁺, and Zn²⁺) were used as crosslinkers, which resulted in a series of polymers (e.g., elastomers or the like) with various mechanical properties and degradability. Other metal ions, such as, for example, Ni, Mn, Ti, or the like, may also be used. It is considered that selection of metal ion(s) can be made to provide polymers (e.g., elastomers, or the like) with specific mechanical properties and/or degradation rates.

As an illustrative example, a chelation crosslinked polymer is made as follows. A Schiff-base ligand (e.g., 2-[[(2-Hydroxyphenyl)amino]-1,3-propanedio, HPA) is synthesized and polycondensed with sebacic acid, 1, 3-propanediol to produce polyester prepolymers. A series of metal ions (e.g., biologically relevant metal ions) (e.g., Cu²⁺, Fe³⁺, Ca²⁺, Co²⁺, Mg²⁺, Zn²⁺, or the like) are mixed with the as-prepared polyester prepolymers. To prepare the polymer films (e.g., elastomer films or the like), the mixtures are cast on a substrate (e.g., glass slides and the like) and cured (e.g., at 30 mTorr and 150° C. for 4 hours) and then cooled (e.g., down to room temperature for 3 hours). To prepare porous polymer scaffolds (e.g., elastomer scaffolds or the like), salt particulates (e.g., NaCl particulates (32˜53 μm), which may be referred to as porogens, are directly added into the prepolymer-metal ions mixtures (e.g., at a prepolymer/salt mass ratio=1:3), and the obtained pastes transferred into a silicone mold. The pastes are cured (e.g., at 30 mTorr and 150° C. for 4 hours) and then cooled (e.g., to room temperature). The salt particulates are removed by immersing the samples in deionized water (e.g., for 48 hours with replacement the deionized water every 6 hours). The porous scaffolds are then freeze-dried prior to use.

In an aspect, the present disclosure provides chelation crosslinked polymers. Non-limiting examples of chelation crosslinked polymers are provided herein. In various examples, a chelation crosslinked polymer is made by a method of the present disclosure.

A chelation crosslinked polymer can comprise various structures. In various examples, a chelation crosslinked polymer (e.g., a chelation crosslinked polyester or the like) is a chelation crosslinked oligomer, a chelation crosslinked prepolymer, a chelation crosslinked homopolymer, a chelation crosslinked copolymer, or the like, or a combination thereof. In various examples, a chelation crosslinked polymer is a chelation crosslinked polyester. In various examples, a chelation crosslinked polyester is a chelation crosslinked polyester oligomer, a chelation crosslinked polyester prepolymer, a chelation crosslinked polyester homopolymer, a chelation crosslinked polyester copolymer, or the like, or a combination thereof.

In various examples, a chelation crosslinked polymer (e.g., a chelation crosslinked polyester or the like) comprises a polymer backbone (e.g., a polyester backbone or the like) containing one or more repeat group(s) (e.g., ester group(s) or the like). In various examples, a chelation crosslinked polyester comprises a polyester backbone containing one or more ester group(s). In various examples, a polymer backbone (e.g., a polyester backbone or the like) comprises one or more chelation crosslinking group(s) within and/or pendant from the polymer backbone (e.g., the polyester backbone or the like). In various examples, a polyester backbone comprises one or more chelation crosslinking group(s) within and/or pendant from the polyester backbone.

In various examples, a chelation crosslinked polymer (e.g., a chelation crosslinked polyester or the like) further comprises one or more cation(s). In various examples, a chelation crosslinked polyester further comprises one or more cation(s). In various examples, at least a portion of cation(s) is/are bonded to at least a portion of chelation crosslinking group(s). In various examples, said bonding thereby crosslinks the polymer backbone (e.g., a polyester backbone or the like). In various examples, said bonding thereby crosslinks a polyester backbone.

In various examples, a chelation crosslinked polyester comprises a polyester backbone comprising one or more ester group(s), wherein the polyester backbone comprises one or more chelation crosslinking group(s) within and/or pendant from the polyester backbone; and one or more cation(s), wherein at least a portion of the cation(s) is/are bonded to at least a portion of the chelation crosslinking group(s) via one or more chelation crosslinking bond(s), thereby crosslinking the polyester backbone. In various examples, 0.01 mol % to 50 mol % of the ester group(s) comprise a chelation crosslinking group.

In various examples, one or more (or all) chelation crosslinking group(s) is/are pendant from a polymer backbone (e.g., a polyester backbone or the like). In various examples, at least one or more or all of the chelation crosslinking group(s) is/are pendant from the polyester backbone.

In various examples, one or more repeat unit(s) (e.g., ester group(s) or the like) comprise(s) one or more chelation crosslinking group(s). In various examples, one or more ester group(s) comprise(s) one or more chelation crosslinking group(s). Various mole ratios of chelation crosslinking group(s) to ester group(s) can be used. In various examples, 0.01 mol % to 100 mol % (e.g., 0.01 mol % to 50 mol %) of repeat group(s) (e.g., ester group(s) or the like), including all 0.01 mol % values and ranges therebetween, comprise a chelation crosslinking group. In various examples, 0.01 mol % to 100 mol % (e.g., 0.01 mol % to 50 mol %) of the ester group(s), including all 0.01 mol % values and ranges therebetween, comprise a chelation crosslinking group. In various examples, 0.01 mol % to 50 mol % of the ester group(s) comprise a chelation crosslinking group.

In various examples, the polymer backbone (e.g. a polyester backbone or the like) is an aliphatic polymer backbone (e.g., an aliphatic polyester backbone or the like) and one or more (or all) repeat group(s) (e.g., ester group(s) or the like) are aliphatic repeat group(s) (e.g., aliphatic ester group(s) or the like). In various examples, the aliphatic repeat group(s) (e.g., ester group(s) or the like) of an aliphatic polymer backbone (e.g., an aliphatic polyester backbone or the like), comprise(s) one or more C₁ to C₂₀ alkyl group(s) (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl group(s), or the like, or combinations thereof). In various examples, the polyester backbone is an aliphatic polyester backbone, and wherein one or more or all of the ester group(s) is/are aliphatic ester group(s). In various examples, the aliphatic ester group(s) of an aliphatic polyester backbone comprise(s) one or more C₁ to C₂₀ alkyl group(s) (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ group(s), or the like, or combinations thereof).

A chelation crosslinked polymer can comprise various types of bonding between chelation crosslinking group(s) and cation(s). In various examples, at least a portion of (or all) cation(s) is/are bonded to at least a portion of (or all) chelation crosslinking group(s) via bonds chosen from ionic bonds, coordinate covalent bonds, and the like, and combinations thereof. In various examples, individual cation(s) is/are bonded to two or more chelation crosslinking group(s). In various examples, crosslinking is reversible. Various mole ratios of chelation crosslinking group(s) to cation(s) can be used. In various examples, the mole ratio of the chelation crosslinking group(s) to the cation(s) is 1:1 to 6:1.

A chelation crosslinked polymer can comprise various other functional group(s). In various examples, a polymer backbone (e.g., a polyester backbone or the like) further comprises one or more other functional group(s) enabling one or more inter- and/or other intra-chain bond(s) other than chelation crosslinking bond(s). In various examples, a polyester backbone further comprises one or more functional group(s) enabling one or more inter- and/or intra-chain bond(s) other than chelation crosslinking bond(s). In various examples, functional group(s) are chosen from amide groups, carboxylate groups, hydroxyl groups, other hydrogen bonding functional groups (e.g., nucleic acid bases or the like), and functional group(s) capable of host-guest chemistry (e.g., cyclodextrin, adamantane or the like), and the like, and combinations thereof.

A chelation crosslinked polymer can comprise various other types of bonds other than chelation crosslinking bonds. In various examples, said inter- and/or other intra-chain bond(s) are chosen from hydrogen bonds, non-polar interactions, salt bridges, pi-pi bonds, and the like, and combinations thereof. In various examples, a chelation crosslinked polymer (e.g., a chelation crosslinked polyester or the like) is crosslinked by said inter- and/or intra-chain bond(s)). In various examples, a chelation crosslinked polyester is crosslinked by said inter- and/or intra-chain bond(s). In various examples, said crosslinking is reversible. In various examples, one or more (or all) chelation crosslinking bond(s) and/or one or more (or all) other bond(s) is/are reversible. In various examples, one or more (or all) chelation crosslinking bond(s) and/or one or more (or all) other bond(s) is/are nonreversible.

In various examples, a chelation crosslinked polymer (e.g., a chelation crosslinked polyester or the like) is a gel polymer (e.g., a hydrogel or the like). In various examples, a chelation crosslinked polymer (e.g., a chelation crosslinked polyester or the like) is a dry polymer (e.g., not a hydrogel or the like). In various examples, a chelation crosslinked polymer (e.g. an isolated chelation crosslinked polymer or the like) (e.g. a chelation crosslinked polyester or the like) comprises 50% or less, 40% or less, 30% or less, 20% or less, 15% or less, 10% or less, 5% or less, 1% or less, or 0.1% or less water by weight (based on the total weight of the chelation crosslinked polymer (e.g., the chelation crosslinked polyester or the like) and water). In an example, a chelation crosslinked polymer (e.g., an isolated chelation crosslinked polymer or the like) (e.g., a chelation crosslinked polyester or the like) is anhydrous. In various examples, a chelation crosslinked polyester (e.g., an isolated chelation crosslinked polyester or the like) is a hydrogel, optionally, having 50% or less water by weight (based on the total weight of the chelation crosslinked polyester and water).

In various examples, a chelation crosslinked polymer (e.g., a chelation crosslinked polyester or the like) comprises a copolymer backbone (e.g., a polyester copolymer backbone or the like). In various examples, a chelation crosslinked polyester comprises a polyester copolymer backbone. Various copolymers backbones can be used. In various examples, a copolymer backbone is a block copolymer backbone comprising one or more other block(s) (other block(s) are block(s) other than polymer backbone blocks(s) (e.g., a polyester backbone block(s) or the like) chosen from one or more other hydrophilic block(s), one or more other hydrophobic block(s), and the like, and combinations thereof. In various examples, a polyester copolymer backbone comprises one or more other block(s) (other block(s) are block(s) other than polyester backbone block(s)) chosen from one or more other hydrophilic block(s), one or more other hydrophobic block(s), and the like, and combinations thereof.

In various examples, other hydrophilic block(s) is/are chosen from polyethylene glycol (PEG) blocks, polylactic acid (PLA) blocks, poly(acrylic acid) (PAA) blocks, and the like, and combinations thereof; and/or the hydrophobic block(s) is/are chosen from polyethylene terephthalate (PET) blocks, poly(caprolactone) (PCL) blocks, poly(methyl methacrylate) (PMMA) blocks, and the like, and combinations thereof.

A chelation crosslinked polymer can comprise various polymer backbone (e.g., polyester backbone or the like) or copolymer backbone (e.g., polyester copolymer backbone or the like) structures. In various examples, a polymer backbone is a polyester backbone having the following homopolymer or copolymer structure:

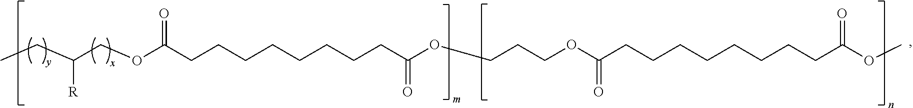

wherein R is a pendant chelation crosslinking group, m is 0.01 to 100 and n is 0 to 99.99. In various examples, all R groups are structurally the same. In various examples, one or more (or all) R groups are structurally different from one or more (or all) other R groups. In various examples, m is 0.01 to 100 (e.g., 0.01 to 50), including all 0.01 values and ranges therebetween, and n is 0 to 99.99 (e.g. 50 to 99.99), including all 0.01 values and ranges therebetween.

A chelation crosslinked polymer (e.g., a chelation crosslinked polyester or the like) can comprise various chelation crosslinking group(s). In various examples, chelation crosslinking group(s) is/are chosen from polydentate chelation crosslinking groups, and the like, and combinations thereof. In various examples, chelation crosslinking group(s) is/are chosen from bidentate, tridentate, tetradendate, and pentadentate chelation crosslinking groups, and the like, and combinations thereof.

In various examples, a chelation crosslinking group comprises or a plurality of or all the chelation crosslinking groups each comprise one or more nitrogen donor group(s), one or more oxygen donor group(s), or the like, or a combination thereof. In various examples, chelation crosslinking group(s) is/are chosen from imine groups, carboxylate groups, aromatic heterocycle groups, amine groups, hydroxyl groups, ether groups, polyether groups, and crown ether groups, and the like, and combinations thereof. In various examples, chelation crosslinking group(s) is/are chosen from salicylaldimine groups, 2-vanillin groups, 2,3-dihydroxybenzaldehyde groups, 2,4-pyridinedicarbonyl dichloride groups, 2-[[3,4-bis[(triethylsilyl)oxy]phenyl]methyl]-oxirane groups, and [2,2′-Bipyridine]-5,5′-dicarbonyl dichloride groups, and the like, and combinations thereof.

A chelation crosslinked polymer (e.g., a chelation crosslinked polyester or the like) may comprise one or more histidine group(s). In various examples, a histidine group is derived from (or formed from) a histidine amino acid.

A chelation crosslinked polymer can comprise various cation types and charges. In various examples, cation(s) is/are chosen from Group(II) cations, transition metals, and the like, and combinations thereof. In various examples, transition metals are chosen from first row transition metals, and the like, and combinations thereof. In various examples, cation(s) is/are present at 0.01% to 100% (e.g., 0.01% to 50%) by weight, including all 0.01 values and ranges therebetween, based on the total weight of a polymer backbone (e.g., a polyester backbone or the like) and cation(s).

A polymer backbone (e.g., a polyester backbone or the like) can exhibit various properties. In various examples, a polymer backbone (e.g., a polyester backbone or the like) exhibits a glass transition temperature (T_{g}) below room temperature. In various examples, a polymer backbone (e.g., a polyester backbone or the like) is semicrystalline. In various examples, a polyester backbone exhibits a glass transition temperature (T_{g}) below room temperature. In various examples, a polyester backbone is semicrystalline.

A polymer backbone (e.g., a polyester backbone or the like) can comprise various end group(s). In various examples, a polymer backbone (e.g., a polyester backbone or the like) comprises end group(s) chosen from acid groups, carboxylate groups, alcohol groups, ester groups, and amide groups, and derivatives thereof, and the like, and combinations thereof. In various examples, a polyester backbone comprises end group(s) chosen from acid group(s), carboxylate group(s), alcohol group(s), ester group(s), and amide group(s), and derivative(s) thereof, and the like, and combinations thereof.

A polymer backbone (e.g., a polyester backbone or the like) can comprise various molecular weight and/or polydispersity index values. In various examples, a polymer backbone (e.g., a polyester backbone or the like) has a molecular weight (M_{w} and/or Mₙ) of 1,000 to 10,000,000 g/mol, including all 1 g/mol values and ranges therebetween, and/or a polydispersity index of 1 to 5, including all 0.1 values and ranges therebetween. In various examples, a polyester backbone has a molecular weight (M_{w} and/or Mₙ) of 1,000 to 10,000,000 g/mol, including all 1 g/mol values and ranges therebetween, and/or a polydispersity index of 1 to 5, including all 0.1 values and ranges therebetween.

A chelation crosslinked polymer (e.g., a chelation crosslinked polyester or the like) can exhibit various properties. In various examples, a chelation crosslinked polymer (e.g., a chelation crosslinked polyester or the like) exhibits one or more (or all) of the following: biocompatibility; biodegradability; a porosity of 60% or greater; a hysteresis of 100% or less; a contact angle of 53° to 83°; a strain of break of from 130% to 520%; a Young's modulus of from 0.5 MPa to 4 MPa; an ultimate tensile strength (UTS) of from 1485 kPa to 2300 kPa; a water content of 0% to 50% by weight, based on the total weight of a polymer (e.g., a polyester or the like) and water. In various examples, a chelation crosslinked polyester exhibits one or more (or all) of the following: biocompatibility; biodegradability; a porosity of 60% or greater; a hysteresis of 100% or less; a contact angle of 53° to 83°; a strain of break of from 130% to 520%; a Young's modulus of from 0.5 MPa to 4 MPa; an ultimate tensile strength (UTS) of from 1485 kPa to 2300 kPa; a water content of 0% to 50% by weight, based on the total weight of a polyester and water.

In various examples, a chelation crosslinked polymer (e.g., a chelation crosslinked polyester or the like) may comprise one or more functional group(s) (e.g., hydroxyl group(s), carboxylic acid group(s), or the like, or a combination thereof) located, e.g., in end group(s), chelation crosslinking group(s), polymer backbone group(s), or the like, which can provide sites to which molecules may be attached to modify the bulk or surface properties of the polymer (Jayachandran, K. N., et al., Synthesis of Dense Brush Polymers with Cleavable Grafts. Eur. Polym. J. 36: 743-749, 2000; Laschewsky, A., et al, Tailoring of Stimuli-responsive Water Soluble Acrylamide and Methacrylamide Polymers. Macromol. Chem. Phys. 202: 276-286, 2001).

For example, tert-butyl, benzyl, or other hydrophobic groups may be added to a chelation crosslinked polymer (e.g., a chelation crosslinked polyester or the like) to reduce the degradation rate of the polymer. Polar organic groups such as, for example, methoxy groups and the like, may also facilitate adjustment of both the degradation rate and hydrophilicity. In contrast, addition of hydrophilic groups, for example, sugars and the like, at these sites may increase the degradation rate. Acid groups may also be added to a chelation crosslinked polymer (e.g., a chelation crosslinked polyester or the like), to modify the properties. For example, molecules with carboxylic or phosphoric acid groups or acidic sugars or the like may be added. Charged groups such as, for example, sulfates, amines, and the like may also be attached to a chelation crosslinked polymer (e.g., a chelation crosslinked polyester or the like). Groups that are added to a chelation crosslinked polymer (e.g., a chelation crosslinked polyester or the like) may be added via linkage to a functional group (e.g., a carboxylate groups or a carboxylic acid group or a hydroxyl group (substituting for hydrogen) or the like), linked directly to the backbone of a chelation crosslinked polymer (e.g., a chelation crosslinked polyester, or the like), incorporated into an organic group which is linked to a chelation crosslinked polymer (e.g., a chelation crosslinked polyester or the like), or the like. For example, a charged amino acid such as, for example, arginine, histidine, or the like may be attached to modify the degradation rate.

Attachment of such non-protein organic or inorganic groups modifies the hydrophilicity and the degradation rate and mechanism of a chelation crosslinked polymer (e.g., a chelation crosslinked polyester or the like). Protecting group chemistry may also be used to modify the hydrophilicity of a chelation crosslinked polymer (e.g., a chelation crosslinked polyester or the like). One skilled in the art will recognize that a wide variety of non-protein organic and inorganic groups may be added to or substituted for the functional groups in a chelation crosslinked polymer (e.g., a chelation crosslinked polyester or the like) to modify its properties. Exemplary functional groups are also described in March, Advanced Organic Chemistry. Fifth edition, John Wiley & Sons, Inc., New York, 1995, the contents of which with regard to functional groups and related chemistry are incorporated by reference herein.

To further control or regulate interaction of a chelation crosslinked polymer (e.g., a chelation crosslinked polyester, or the like) with cells, biomolecules, small molecules, bioactive agents, or the like may be coupled to the functional groups or otherwise integrated into the polymer backbone or chelation crosslinking group(s) of a chelation crosslinked polymer (e.g., a chelation crosslinked polyester or the like) (Barrera, D., et al., Synthesis and RGD Peptide Modification of a New Biodegradable Copolymer: Poly(lactic acid-co-lysine). J. Am. Chem. Soc. 115: 11010-11, 1993; West, J. L., et al., Polymeric Biomaterials with Degradation Sites for Proteases Involved in Cell Migration. Macromolecules 32: 241-244, 1999; Mann, B. K., Smooth Muscle Cell Growth in Photopolymerized Hydrogels with Cell Adhesive and Proteolytically Degradable Domains: Synthetic ECM Analogs for Tissue Engineering. Biomaterials 22, 3045-3051; 2001). Biomolecules, small molecules, bioactive agents, or the like, or a combination thereof which are encapsulated within a chelation crosslinked polymer (e.g., chelation crosslinked polyester or the like), perhaps linked to it using non-covalent interactions, but not attached to the chelation crosslinked polymer (e.g., a chelation crosslinked polyester, or the like) may diffuse out of a chelation crosslinked polymer prior to degradation of a chelation crosslinked polymer (e.g., the chelation crosslinked polyester or the like). In contrast, attachment of biomolecules, small molecules, bioactive agents, or the like to a chelation crosslinked polymer (e.g., a chelation crosslinked polyester, or the like) may result in a slower release rate because biomolecules, small molecules, or bioactive agents may not be released from the material until it degrades.

For example, biomolecules, such as, for example, growth factors may be exploited to recruit cells to a wound site or promote specific metabolic or proliferative behavior in cells that are at the site or seeded within the matrix. Exemplary growth factors include, but are not limited to, TGF-β, acidic fibroblast growth factor, basic fibroblast growth factor, epidermal growth factor, IGF-I and II, vascular endothelial-derived growth factor, bone morphogenetic proteins, platelet-derived growth factor, heparin-binding growth factor, hematopoetic growth factor, peptide growth factor, and the like. Integrins and cell adhesion sequences (e.g., the RGD sequence and the like) may be attached to the chelation crosslinked polymers (e.g., chelation crosslinked polyesters, or the like) to facilitate cell adhesion. Integrins are part of a large family of cell adhesion receptors that are involved in cell-extracellular matrix and cell-cell interactions. The RGD sequence, present in proteins such as, for example, fibronectin and the like, has been shown to be active in promoting cell adhesion and proliferation (Massia, et al., J. Cell. Biol. 114:1089, 1991). Extracellular matrix components, e.g., collagen, fibronectin, laminin, elastin, etc., may be combined with a chelation crosslinked polymer (e.g., a chelation crosslinked polyester or the like) to manipulate cell recruitment, migration, metabolism, or the like or a combination thereof and/or the degradation properties, mechanical properties or the like, or a combination thereof of the material. Proteoglycans and glycosaminoglycans may also be covalently or non-covalently attached to a chelation crosslinked polymer (e.g., a chelation crosslinked polyester, or the like).

As used herein, the term “pore” refers to a minute opening in a surface through which gases, liquids, or solid materials of the requisite dimension can pass. In various examples, a pore is an opening in a chelation crosslinked polymer (e.g., a chelation crosslinked polyester), or a composition, a fiber, a material, or a tissue graft thereof. In various examples, a pore is an opening formed between two or more chelation crosslinked polymer(s) (e.g., chelation crosslinked polyester(s)) or composition(s), fiber(s), material(s), or a tissue graft(s) thereof. As used herein, the term “porosity” refers to the % by volume of pores based on the total volume of a substrate, especially a chelation crosslinked polymer (e.g., a chelation crosslinked polyester), or a composition, a fiber, a material, or a tissue graft thereof. In various examples, the porosity of the chelation crosslinked polymer (e.g., the chelation crosslinked polyester or the like) is 50% or greater, 55% or greater, 60% or greater, or 65% or greater. In various examples, the porosity of the chelation crosslinked polyester is 50% or greater, 55% or greater, 60% or greater, or 65% or greater.

In some examples, a chelation crosslinked polymer (e.g., a chelation crosslinked polyester or the like) does not comprise any pores. In various examples, the porosity of a chelation crosslinked polymer (e.g., a chelation crosslinked polyester or the like) is an open pore structure. In various examples, the porosity of a chelation crosslinked polymer (e.g., a chelation crosslinked polyester or the like) is a closed pore structure. In various examples, the porosity of a chelation crosslinked polymer (e.g., a chelation crosslinked polyester or the like) is a continuous pore structure. In various examples, the pores of a chelation crosslinked polymer (e.g., a chelation crosslinked polyester or the like) are uniformly distributed. In various examples, the pores of a chelation crosslinked polymer (e.g., a chelation crosslinked polyester or the like) are non-uniformly distributed.

In various examples, the pores of the chelation crosslinked polymer (e.g., the chelation crosslinked polyester or the like) are not interconnected or comprise various amounts of interconnectivity. In some examples, a chelation crosslinked polymer (e.g., a chelation crosslinked polyester or the like) comprises at least 75% pore interconnectivity, such as, for example, about 80% to about 90%, about 90% to about 98%, including 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% pore interconnectivity.

Various polymerization reactions can form a polymer backbone (e.g., a polyester backbone or the like) having a plurality of repeat group(s) (e.g., ester group(s) or the like). In various examples, the polymer backbone (e.g., a polyester backbone or the like) is formed via addition polymerization or condensation polymerization. In various examples, one or more various cyclic monomer(s) (e.g., lactone(s) or the like) undergo(es) ring opening polymerization to form one or more repeat group(s) (e.g., ester group(s) or the like). In various examples, one or more pair(s) of complementary monomer(s) (e.g., polyacid(s) and polyol(s) or the like) undergo(es) condensation polymerization to form one or more repeat group(s) (e.g., ester group(s) or the like). In various examples, the polyester backbone is formed via addition polymerization or condensation polymerization. In various examples, one or more ester group(s) are formed from ring opening polymerization of one or more lactone(s) which, optionally, comprise(s) one or more chelation crosslinking group(s). In various examples, one or more ester group(s) are formed from condensation of one or more pair(s) of polyacid(s) and polyol(s) which, optionally, comprise(s) one or more chelation crosslinking group(s).

In various examples, the polyester backbone is formed from condensation of one or more polyacid(s) (such as, for example, diacid(s), triacid(s) or the like) and one or more polyol(s) (such as, for example, diol(s), triol(s) or the like), and at least a portion (or all) of the one or more polyacid(s) and/or one or more polyol(s) comprise(s) one or more chelation crosslinking group(s). A polyester backbone may comprise one or more ester group(s) formed from reaction of one or more of these polyacid(s) and/or polyol(s).

Non-limiting examples of polyacids include citric acid, succinic acid, disulfuric acid, pyromellitic acid or the like, and derivatives thereof, and combinations thereof. Non-limiting examples of diacids include sebacic acid, glutamic acid (e.g., L-glutamic acid and the like), succinic acid, adipic acid, suberic acid, malonic acid, glutaric acid, azelaic acid or the like, and derivatives thereof, and combinations thereof.

Non-limiting examples of polyols include glycerol, sorbitol, mannitol, xylitol, maltitol, maltitol syrup, lactitol, erythritol or the like, and derivatives thereof, and combinations thereof. Non-limiting examples of diols include propane diols (e.g., 1,3-propane diol, 1,4-butane diol or the like), ethylene diols (e.g., a hydroxyl terminated oligo ethylene, polyethylene glycol or the like) or the like, and derivatives thereof, and combinations thereof.

In an aspect, the disclosure provides chelation polymers (e.g., chelation polyesters or the like). In various examples, a chelation polymer (e.g., a chelation polyester or the like) comprises a polymer backbone (e.g., a polyester backbone or the like) of the present disclosure but not one or more cation(s) of the present disclosure, thus crosslinking does not occur. In various examples, a chelation polymer comprises a polymer backbone (e.g., a polyester backbone or the like) and one or more cation(s) of the present disclosure, but the cation(s) is/are not bonded to the chelation crosslinking group(s), thus crosslinking does not occur.

In various examples, a chelation crosslinked polymer (e.g., a chelation crosslinked polyester or the like) is not covalently crosslinked (e.g., does not comprise one or more covalent crosslinking bond(s)). In various examples, a chelation crosslinked polyester is not covalently crosslinked (e.g., does not comprise one or more covalent crosslinking bond(s)).

In an aspect, the present disclosure provides compositions. Non-limiting examples of compositions include scaffolding materials, particles, such as, for example, beads, microspheres, nanospheres and the like, surface coatings, structural materials, composites or the like. Non-limiting examples of compositions are provided herein. In various examples, a composition is made by a method of the present disclosure.

In various examples, a composition comprises one or more chelation crosslinked polymer(s) (e.g., chelation crosslinked polyester(s) or the like) of the present disclosure. In various examples, a composition comprises one or more chelation crosslinked polyester(s).

In various examples, a composition is a three dimensional (3D) object. In various examples, a composition is a biomedical composition, a pharmaceutical composition, a chewing gum base, a sealant, or the like. In various examples, a composition is a fiber, a film, a monolith, a tube, a foam, or the like.

In various examples, a pharmaceutical composition further comprises one or more active ingredient(s) and, optionally, one or more excipient(s) and/or pharmaceutical carrier(s). In various examples, a pharmaceutical composition is suitable for diagnostic, therapeutic, or preventive use.

As used herein, “pharmaceutically acceptable carrier or excipient” refers to a carrier or excipient that is useful in preparing a pharmaceutical formulation that is generally safe, non-toxic, and is neither biologically undesirable nor otherwise undesirable, and includes a carrier or excipient that is acceptable for veterinary pharmaceutical use human pharmaceutical use, or both. A “pharmaceutically acceptable carrier or excipient” includes both one and more than one such carrier or excipient. One or more of the chelation crosslinked polymer(s) (e.g., chelation crosslinked polyester(s) or the like) and or one or more of the active ingredient(s) may be present as a pharmaceutically acceptable salt.

As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, pamoate, and the like, and combinations thereof.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” refers generally to obtaining a desired pharmacological and/or physiological effect. The effect can be, but does not necessarily have to be, prophylactic in terms of preventing or partially preventing a disease, symptom, or condition thereof, such as, for example, a proliferative disease, or the like. The effect can be therapeutic in terms of a partial or complete cure of a disease, condition, symptom or adverse effect attributed to the disease, disorder, or condition. The term “treating” as used herein covers any disease, symptom, or condition thereof, in a subject (e.g., a human or a non-human animal) and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treating” as used herein can refer to both therapeutic treating alone, prophylactic treating alone, or both therapeutic and prophylactic treating. Those in need of treating (subjects in need thereof) can include those already with the disorder and/or those in which the disorder is to be prevented.

Chelation crosslinked polymers (e.g., chelation crosslinked polyesters, or the like) may be combined with other polymers in blends or adducts to, for example, manipulate the degradation properties, mechanical properties, and the like, and combinations thereof of the material. Practically any biocompatible polymer may be combined with chelation crosslinked polymers (e.g., chelation crosslinked polyesters, or the like). In various examples, the added polymer is biodegradable. Exemplary biodegradable polymers include natural polymers and their synthetic analogs, including polysaccharides, proteoglycans, glycosaminoglycans, collagen-GAG, collagen, fibrin, and other extracellular matrix components, such as, for example, elastin, fibronectin, vitronectin, laminin, and the like. Hydrolytically degradable polymers known in the art include, for example, certain polyesters, polyanhydrides, polyorthoesters, polyphosphazenes, polyphosphoesters, and the like, and combinations thereof. Biodegradable polymers known in the art, include, for example, certain polyhydroxyacids, polypropylfumerates, polycaprolactones, polyhydroxyalkanoates, poly(amide-enamines), polyamides, poly(amino acids), polyacetals, polyethers, biodegradable polycyanoacrylates, biodegradable polyurethanes and polysaccharides, and the like, and combinations thereof. For example, specific biodegradable polymers that may be used include but are not limited to, polylysine, poly(lactic acid) (PLA), poly(glycolic acid) (PGA), copolymers and mixtures of PLA and PGA, e.g., poly(lactide-co-glycolide) (PLG), poly(caprolactone) (PCL), poly(lactide-co-caprolactone) (PLC), and poly(glycolide-co-caprolactone) (PGC). Those skilled in the art will recognize that this is an exemplary, not comprehensive, list of biodegradable polymers. The properties of these and other polymers and methods for preparing them are further described in the art. See, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. Nos. 4,806,621; 4,638,045 to Kohn; and 4,946,929 to d'Amore; see also Wang et al., J. Am. Chem. Soc. 123:9480, 2001; Lim et al., J. Am. Chem. Soc. 123:2460, 2001; Langer, Acc. Chem. Res. 33:94, 2000; Langer, J. Control Release 62:7, 1999; and Uhrich et al., Chem. Rev. 99:3181, 1999.

Chelation crosslinked polymers (e.g., chelation crosslinked polyesters, or the like) may also be combined with non-biodegradable polymers. For example, polypyrrole, polyanilines, polythiophene, derivatives thereof, and the like are useful electrically conductive polymers that can provide additional stimulation to seeded cells or neighboring tissue. Exemplary non-biodegradable polymers include, but are not limited to, polystyrene, polyesters, non-biodegradable polyurethanes, polyureas, poly(ethylene vinyl acetate), polypropylene, polymethacrylate, polyethylene, polycarbonates, poly(ethylene oxide), and the like, and combinations thereof. In various example, one or more chelation crosslinked polymer(s) (e.g., chelation crosslinked polyester(s), or the like) are combined with biodegradable polymer, non-biodegradable polymers, or both.

Alternatively or in addition, fibers and particles may be combined with the chelation crosslinked polymers (e.g., chelation crosslinked polyesters, or the like), for example, to modify its mechanical properties or the like. For example, fibers, e.g., of collagen or PLGA, are be embedded in the chelation crosslinked polymers (e.g., chelation crosslinked polyesters, or the like) for example, to stiffen it. For example, particles of Bioglass™ or calcium phosphate ceramics are combined with the chelation crosslinked polymers (e.g., chelation crosslinked polyesters, or the like).

In various examples, chelation crosslinked polymers (e.g., chelation crosslinked polyesters, or the like) are be used as a base for chewing gum. For example, chelation crosslinked polymers (e.g., chelation crosslinked polyesters, or the like) are combined with a colorant, flavor enhancer, or other additive to produce a gum. The appropriate microstructure to produce a pleasant mouthfeel during chewing can be easily determined by polymerizing the chelation crosslinked polymers (e.g., chelation crosslinked polyesters, or the like) to different molecular weights and cross-link densities and chewing the resulting material for a few minutes.

The gum may also be adapted to deliver nutrients (e.g., vitamins and the like), drugs, or the like to the chewer. Nutrients include, but are not limited to, FDA-recommended nutrients such as, for example, vitamins and minerals, amino acids, various nutritional supplements available at health food stores, and the like and combinations thereof. Such additives may simply be mixed with the chelation crosslinked polymers (e.g., chelation crosslinked polyesters, or the like) to produce a gum. Alternatively, they may be covalently attached to the chelation crosslinked polymers (e.g., chelation crosslinked polyesters, or the like), for example, through hydrolyzable bonds, bonds that are lysed by the enzymes found in the mouth, or the like, or a combination thereof. In this case, as the gum is chewed, for example, the nutrient(s) or drug(s) is released and swallowed. If the gum is swallowed, it will be completely metabolized in the digestive system.

Chelation crosslinked polymers (e.g., chelation crosslinked polyesters, or the like) may also be used for drug release applications, for example, in applications where the matrix retaining the drug needs to be flexible. Biomolecules, small molecules, and bioactive agents may all be combined with chelation chelation crosslinked polymers (e.g., chelation crosslinked polyesters, or the like) of the disclosure using covalent or non-covalent interactions. Exemplary non-covalent interactions include, but are not limited to, hydrogen bonds, electrostatic interactions, hydrophobic interactions, van der Waals interactions, and the like, and combinations thereof.

In some examples, a chelation crosslinked polymer (e.g., chelation crosslinked polyester, or the like), a fiber, a scaffold, or a graft is impregnated with and/or a surface of which is coated with one or more, such as, for example, two, three, four, five etc. suitable pharmaceutical agents. It is contemplated that suitable pharmaceutical agents can be organic or inorganic and may be in a solid, semisolid, liquid, or gas phase. Molecules may be present in combinations or mixtures with other molecules, and may be in solution, suspension, or any other form. Examples of classes of molecules that may be used include, but are not limited to, human or veterinary therapeutics, cosmetics, nutraceuticals, agriculturals such as, for example, herbicides, pesticides and fertilizers, vitamins, salts, electrolytes, amino acids, peptides, polypeptides, proteins, carbohydrates, lipids, nucleic acids, glycoproteins, lipoproteins, glycolipids, glycosaminoglycans, proteoglycans, growth factors, hormones, neurotransmitters, pheromones, chalones, prostaglandins, immunoglobulins, monokines and other cytokines, humectants, metals, gases, minerals, plasticizers, ions, electrically and magnetically reactive materials, light sensitive materials, anti-oxidants, molecules that may be metabolized as a source of cellular energy, antigens, any molecules that can cause a cellular or physiological response, and the like. Any combination of molecules may be used, as well as agonists or antagonists of these molecules.

Pharmaceutical agents include any therapeutic molecule including, but not limited to, any pharmaceutical substance or drug or the like. Examples of pharmaceuticals include, but are not limited to, anesthetics, hypnotics, sedatives and sleep inducers, antipsychotics, antidepressants, antiallergics, antianginal s, antiarthritics, antiasthmatics, antidiabetics, antidiarrheal drugs, anticonvulsants, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfiram and disulfiram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor. All pharmaceutical agents listed by the U.S. Pharmacopeia are also included within the pharmaceutical agents of the present disclosure.

In some examples, the inner luminal surface of a biodegradable scaffold or graft (e.g., vascular graft or the like) is coated partially or completely with a thromboresistant agent, such as, for example, heparin and/or other compounds known to one of skill in the art to have similar anti-coagulant properties as heparin, to prevent, inhibit or reduce clotting within the inner lumen of the biodegradable scaffold or graft (e.g, vascular graft or the like).

Alternatively, one or more biomolecule(s), small molecule(s), bioactive agent(s) or the like may be encapsulated within a chelation crosslinked polymer (e.g., a chelation crosslinked polyester, or the like) and may be linked to it using non-covalent interactions. Attachment of a biomolecule(s), small molecule(s), bioactive agent(s), or the like to a chelation crosslinked polymer (e.g., a chelation crosslinked polyester, or the like) may result in a slower release rate because a biomolecule/biomolecules, small molecule/molecules, bioactive agent/agents, or the like is/are released from the material as it degrades. In contrast, if a biomolecule/biomolecules, small molecule/molecules, bioactive agent/agents is/are encapsulated within a chelation crosslinked polymer (e.g., a chelation crosslinked polyesters, or the like), it may diffuse out of the chelation crosslinked polymers (e.g., chelation crosslinked polyesters, or the like) before the chelation crosslinked polymer (e.g., chelation crosslinked polyester, or the like) degrades (e.g., substantially degrades). In various examples, diffusion of the encapsulated molecules and degradation of the chelation crosslinked polymer occur at the same time.

In an aspect, the present disclosure provides fibers. Non-limiting examples of fibers are provided herein. In various examples, a fiber is made by a method of the present disclosure.

In various examples, a fiber comprises one or more chelation crosslinked polymer(s) (e.g., chelation crosslinked polyester(s) or the like) of the present disclosure. In various examples, a fiber comprises one or more chelation crosslinked polyester(s) of the present disclosure.

In various examples, a fiber comprises a blend of chelation crosslinked polymer(s) (e.g., chelation crosslinked polyester(s) or the like) and one or more other polymer(s) (other polymer(s) are not chelation crosslinked polymer(s) (e.g., chelation crosslinked polyester(s) or the like)) and/or one or more other polymeric material(s) (other polymeric material(s) do not comprise chelation crosslinked polymer(s) (e.g., chelation crosslinked polyester(s) or the like)). In various examples, a fiber comprises a blend of chelation crosslinked polyester(s) and one or more other polymer(s) (other polymer(s) are not chelation crosslinked polyester(s)) and/or one or more other polymeric material(s) (other polymeric material(s) do not comprise chelation crosslinked polyester(s)). Various other polymer(s) and/or other polymeric material(s) can be used. In various examples, other polymer(s) and/or other polymeric material(s) is/are polylactic acids (PLAs), polyglycolic acids (PGAs), PLGAs, poly(caprolactone)s (PCLs), polyethylene glycols (PEGs), polyethylene terephthalates (PETs), polypropylenes, polyethylenes, nylons, polystyrenes, poly(glycerol sebacate) (PGS), poly(methyl methacrylate) (PMMA), poly(acrylic acid) (PAA), or the like, or combinations thereof.

In various examples, a fiber is formed by, for example, electrospinning, wet spinning, melt spinning and other processes that drive polymers through a small orifice or the like. A fiber may comprise one or more other polymer(s) and/or one or more other polymeric material(s).

In an aspect, the present disclosure provides materials. Non-limiting examples of materials are provided herein). In various examples, a material is made by a method of the present disclosure.

In various examples, a material comprises a plurality of fibers of the present disclosure. A material can comprise various forms. In various examples, a material is a fabric or the like. In various examples, a fabric is a weave or braid of fibers or the like. In various examples, a material comprises one or more fibers having chelation crosslinked polymer(s) (e.g., chelation crosslinked polyester(s) or the like) and further comprises one or more other fiber(s) (other fiber(s) do not comprise chelation crosslinked polymer(s) (e.g., chelation crosslinked polyester(s) or the like)). In various examples, a material comprises one or more fibers having chelation crosslinked polyester(s) and further comprises one or more other fiber(s) (other fiber(s) do not comprise chelation crosslinked polyester(s)). Various other fiber(s) can be used. In various examples, other fiber(s) is/are polylactic acid (PLA) fibers, (PCL) fibers, polyethylene glycol (PEG) fibers, PLGA, poly(lactide-co-caprolactone) (PLCL), poly(glycerol sebacate) (PGS), poly(methyl methacrylate) (PMMA), poly(acrylic acid) (PAA) fibers, or the like, or combinations thereof.

In an aspect, the present disclosure provides tissue grafts. Non-limiting examples of tissue grafts are provided herein. In various examples, a tissue graft is made by a method of the present disclosure.

In various examples, a chelation crosslinked polymer (e.g., chelation crosslinked polyester, or the like) is an elastomeric biodegradable polyester. The elasticity of chelation crosslinked polymers (e.g., chelation crosslinked polyesters, or the like) is important for use of the chelation crosslinked polymers (and fibers, materials or the like comprising one or more of the chelation crosslinked polymer(s)) for use in regenerating a variety of tissues. The chelation crosslinked polymers may be used to tissue engineer, for example, epithelial, connective, nerve, muscle, gland, and other tissues and organs. Exemplary tissues and organs that can benefit from the materials of the disclosure include, but are not limited to, blood and lymphatic vessels, ligament, skin, tendon, muscle, heart, lung, kidney, nerve, liver, pancreas, bladder, intestine, and others. Chelation crosslinked polymers (e.g., chelation crosslinked polyesters, or the like) may also be used as the template for, for example, mineralization and formation of bone, or the like. In various examples, chelation crosslinked polymers (e.g., chelation crosslinked polyesters, or the like) of the present are useful for regenerating tissues that are subject to repeated tensile, hydrostatic, or other stresses, such as, for example, lung, blood vessels, heart valve, bladder, cartilage, muscle, and the like.

In some particular examples, the generated tissue constructs are for the replacement and/or repair of damaged native tissues. For example, the disclosed constructs are contemplated to be implantable for tensile load bearing applications, such as, for example, being formed into tubular networks with a finite number of inlets and outlets. These structures can be either seeded with cells or implanted directly and relying on the host to serve as cell source and “bioreactor”. These structures can be implanted as artificial organs and the inlets and outlets will be connected to host tissues, vasculature, and the like. In some examples, the vasculature itself maybe valuable without parenchymal cells. For example, in treating ischemic diseases. The microvascular mimetics can be connected directly to a host vessel and perfuse an ischemic area of the body.

The various dimensions of a disclosed scaffold or vascular graft may vary according to the desired use. In some examples, the method of fabrication is performed to generate a vascular graft with an inner diameter which matches that of the host vessel to be replaced. However, it is contemplated the graft wall can be fabricated with a thicker or thinner wall than that which is being replaced, if desired.

The shape of the chelation crosslinked polymers (e.g., chelation crosslinked polyesters, or the like) may also be manipulated for specific tissue engineering applications. Exemplary shapes include, but are not limited to, particles, tubes, spheres, strands, coiled strands, films, sheets, fibers, meshes, and the like. In various examples, microfabrication is used to form capillary networks from one or more chelation crosslinked polymer(s) (e.g., chelation crosslinked polyester(s), or the like).

The chelation crosslinked polymers (e.g., chelation crosslinked polyesters, or the like) of the present disclosure may be electrospun to form scaffolds of any desired shape, such as, for example, sheets, tubes, meshes, pseudo 3-dimensional constructs, and the like. In various examples, the constructs may be of high porosity, low porosity, or a combination of different porosity. In some examples, the constructs are vascularized (micro-channeled) fibrous sheets, random meshes, aligned sheets, cylindrical tubes, or pseudo 3-dimensional constructs, such as, for example, shapes to mimic organs or the like. Electrospinning with a sacrificial template can be used to create highly porous scaffolds. Porous morphology can be varied. These structures are especially useful for applications in soft and elastomeric tissues.

In various examples, a salt leaching technique may be used to make tubes, disks, or other 3-dimensionals structures to give adapted shape for the use. With salt leaching technique, highly porous scaffolds, with a range of porosity may obtained depending on the salt crystal size and packing methods.

In various examples, a tissue graft comprises one or more chelation crosslinked polymer(s) (e.g., chelation crosslinked polyester(s) or the like) of the present disclosure. In various examples, a tissue graft comprises one or more chelation crosslinked polyester(s) of the present disclosure. In various examples, a tissue graft comprises one or more chelation crosslinked polymer(s) and/or one or more composition(s) comprising one or more chelation crosslinked polymer(s) and/or one or more fiber(s). A tissue graft may comprise (e.g., further comprise) a polymer component (which is not a polyester component, such as, for example, PETE or the like). In various examples, such polymer components are non-biodegradable. In various examples, a tissue graft comprises one or more other fiber(s) (non-chelation crosslinked polymer(s)), and some or all of which may degrade forming a scaffold comprising the remaining fibers.

A tissue graft can comprise various forms. In various examples, a tissue graft is a soft tissue graft or the like. In various examples, a tissue graft is a soft tissue graft (such as, for example, blood vessel grafts, muscle grafts, skin grafts, ligament grafts, internal organs (such as, for example, lungs, kidneys, hearts or the like), nervous system tissue grafts or the like), or the like. In various examples, a soft tissue graft is a vascular graft or the like. In various examples, a vascular graft is an arterial graft or the like. In various examples, an arterial graft comprises a lumen diameter of 6 mm or less.

In some examples, a scaffold or tissue graft includes uniformly distributed pores. In some examples, a scaffold or tissue graft includes non-uniformly distributed pores. In some examples, a scaffold or tissue graft does not include any pores. In some examples, a porous scaffold or porous tissue graft includes at least 75% pore interconnectivity, such as, for example, about 80% to about 90%, about 90% to about 98%, including 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% interconnectivity.

At least a portion or all of a scaffold or tissue graft may degrade after implantation in an individual. In some examples, at least 50%, such as, for example, about 55% to about 70%, about 80% to about 90%, about 90% to about 98%, including 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% of a scaffold or tissue graft (e.g., a vascular graft) degrades within one year, such as, for example, within 1 to 10 months, including within 1 month, 2 months, 3 months, 4 months, 5 months, 6 months, 7 months, 8 months, 9 months, 10 months, 11 months, or 12 months of implantation.

In an aspect, the present disclosure provides articles of manufacture. In various examples, an article of manufacture comprises one or more fiber(s) and/or one or more composition(s) comprising one or more chelation crosslinked polymer(s) and/or one or more chelation crosslinked polymer(s). Non-limiting examples of articles of manufacture are provided herein. In various examples, an article of manufacture is made by a method of the present disclosure.

In various examples, an article of manufacture comprises one or more chelation crosslinked polymer(s) (e.g., chelation crosslinked polyester(s) or the like) of the present disclosure. In various examples, an article of manufacture comprises one or more chelation crosslinked polyester(s) of the present disclosure. An article of manufacture can comprise various forms. In various examples, the article of manufacture is chosen from consumer goods, tires, gloves, gaskets, washers, toys, chewing gum, hoses, and balloons, and the like, and combinations thereof.

The steps of the methods described in the various embodiments and examples disclosed herein are sufficient to produce a polymer of the present disclosure or carry out a method of the present disclosure. Thus, in various embodiments, a method consists essentially of a combination of the steps of the methods disclosed herein. In various other embodiments, a method consists of such steps.

The following are Statements describing examples of the present disclosure and are not intended to be in any way limiting:

Statement 1. A polyester (which may be a chelation crosslinked polyester), which may be an elastomer, comprising: a polyester comprising a backbone comprising one or more (e.g., a plurality of) chelation crosslinking group(s) (e.g., one or more backbone chelation crosslinking group(s) (e.g., chelation crosslinking group(s) in the backbone) and/or one or more pendent chelation crosslinking group(s), or a combination thereof); and one or more (e.g., a plurality of) cation(s), where at least a portion of (or all) of the one or more (e.g., a plurality of) cation(s) is/are bonded (e.g., ionically bonded, coordination (coordinatively covalently) bonded, or the like, or a combination thereof) to at least a portion of (or all) of the chelation crosslinking group(s) (e.g., the backbone chelation crosslinking group(s) and/or the pendent chelation crosslinking group(s)).
Statement 2. A polyester according to Statement 1, where the chelation crosslinking group(s) are chosen, independently at each occurrence, from imine groups, carboxylate groups, aromatic heterocycle groups (such as, for example, pyridines, histidines or the like), amine groups, ether groups, polyether groups, crown ether groups or the like.
Statement 3. A polyester according to Statement 1 or 2, where at 0.01 to 50% (e.g., 50% or less, 40% or less, 30% or less, 20% or less, 15% or less, 10% or less, 5% or less, 1% or less, or 0.1% or less) of the polyester monomer units comprise a chelation crosslinking group (e.g., comprise backbone chelation crosslinking group(s) and/or the pendent chelation crosslinking group(s)).
Statement 4. A polyester according to any one of Statements 1-3, where the cation(s) is/are chosen from Group(II) cations, transition metals (such as, for example, first row transition metals or the like), and the like, and combinations thereof.
Statement 5. A polyester according to any one of the proceeding Statements, where the cation(s) are present at 50% or less, 40% or less, 30% or less, 20% or less, 15% or less, 10% or less, 5% or less, 1% or less, or 0.1% or less by weight (based on the total weight of the polymer and cations).
Statement 6. A polyester according to any one of the preceding Statements, where the polyester is an aliphatic polyester comprising a backbone comprising one or more (e.g., a plurality of) chelation crosslinking group(s) and/or a backbone with one or more (e.g., a plurality of) chelation crosslinking pendent group(s).
Statement 7. A polyester according to any one of the preceding Statements, where the polyester (or its oligomer analog) has a molecular weight (Mw and/or Mn) of 1,000 to 10,000,000 g/mol, including all 0.1 g/mol values and ranges therebetween, and/or a polydispersity index of 1 to 5, including all 0.1 values and ranges therebetween.
Statement 8. A polyester according to any one of the preceding Statements, where in the polyester comprises (or has) the following structure:

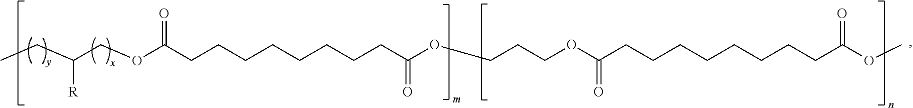

wherein R is a pendant chelating crosslinking group,
m is 0.01 to 50, including all 0.01 values and ranges therebetween, and
n is 50 to 99.99, including all 0.01 values and ranges therebetween.
   Statement 9. A polyester according to any one of the preceding Statements, where the polyester exhibits one or more or all of the following:
Desirable hysteresis (e.g., 100% or less hysteresis; 75% or less hysteresis, 50% or less hysteresis, 25% or less hysteresis, 10% or less hysteresis, or 5% or less hysteresis or the like).
Desirable biocompatibility
Desirable biodegradability
Desirable hydrophilicity (e.g., a contact angel of 53° to 83°, including all 0.1° values and ranges therebetween).
   Statement 10. A polyester according to any one of the preceding Statements, where the polyester is a copolymer comprising a polyester of any of Statements 1-9.
   Statement 11. A composition comprising one or more polyester(s) of the present disclosure (e.g., polyester(s) of any one of Statements 1-10).
   Statement 12. A composition according to Statement 11, where the composition is a three-dimensional object (such as, for example, a fiber, a film, a monolith, a foam or the like).
   Statement 13. A fiber comprising one or more polyester(s) of any one of Statements 1-10, one or more composition(s) of any one of Statements 11 or 12, or a combination thereof.
   Statement 14. A fiber according to Statement 13, where the fiber comprises one or more other polymer(s) and/or one or more other polymeric material(s).
   Statement 15. A material comprising a plurality of one or more fiber(s) of Statement 13 or 14.
   Statement 16. A material according to Statement 15, where the material is a fabric.
   Statement 17. A material according to Statement 15 or 16, where the material comprises one or more other fiber(s).
   Statement 18. A tissue graft comprising one or more polyester(s) of any one of Statements 1-10, one or more composition(s) of any one of Statements 11 or 12, or a combination thereof, and/or a fiber or material comprising one or more polyester(s) of any one of Statements 1-10, one or more compositions(s) of any one of Statements 11 or 12, or a combination thereof (e.g., a fiber or any one of Statements 13 or 14 or a material of any one of Statements 15 to 17, or a combination thereof).
   Statement 19. A tissue graft according to Statement 18, where, in the case of materials comprising one or more other fiber(s), one or more of the other fiber(s) degrades (e.g., biodegrades in an individual) forming a scaffold comprising the remaining fibers.
   Statement 20. A tissue graft according to Statement 18 or 19, where the tissue graft is a vascular graft.
   Statement 21. A tissue graft according to Statement 20, where the vascular is an arterial graft, which may be a small artery graft, or the like.
   Statement 22. An article of manufacture comprising one or more polyester according to any one of Statements 1-10, one or more composition according to Statement 11 or 12, one or more fiber according to any one of Statements 13 or 14, one or more material according to any one of Statements 15-17, or a combination thereof.
   Statement 23. An article of manufacture according to Statement 19, where the article of manufacture is chosen from consumer goods, tires, gloves, gaskets, washers, toys, chewing gum, hoses, and balloons, and the like, and combinations thereof.

The following example is presented to illustrate the present disclosure. The example is not intended to be limiting in any matter.

### EXAMPLES

### Example 1. Chelation Crosslinking of Elastomers

The following is an example of methods and chelation crosslinked polymers of the present disclosure.

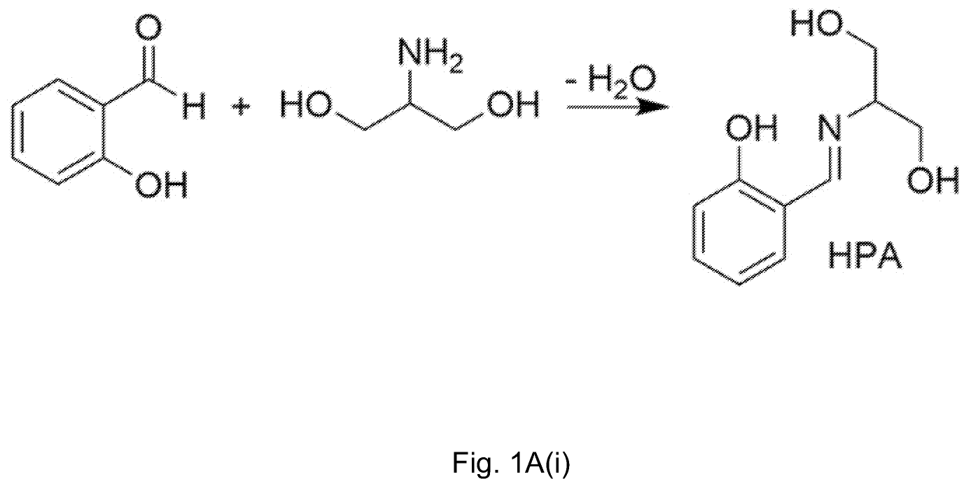

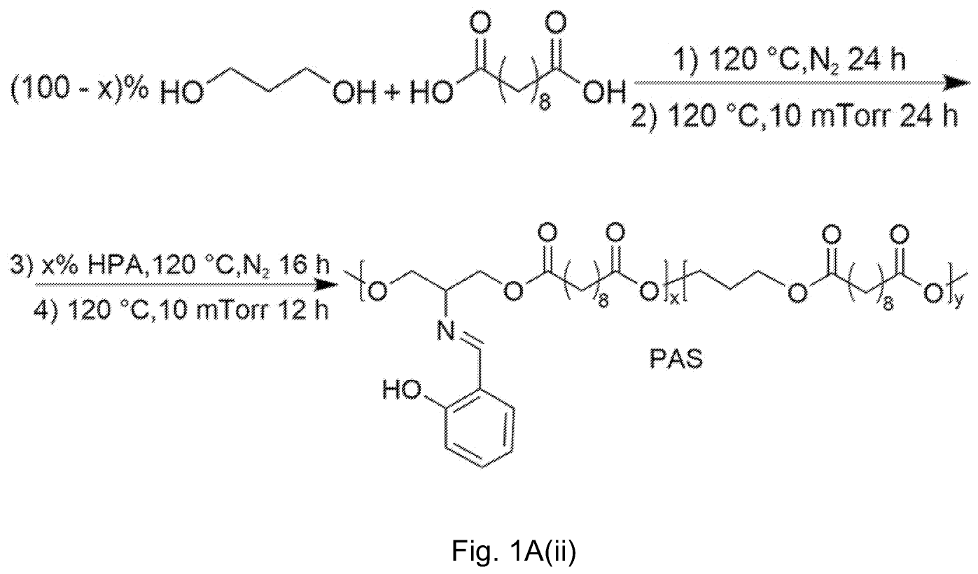

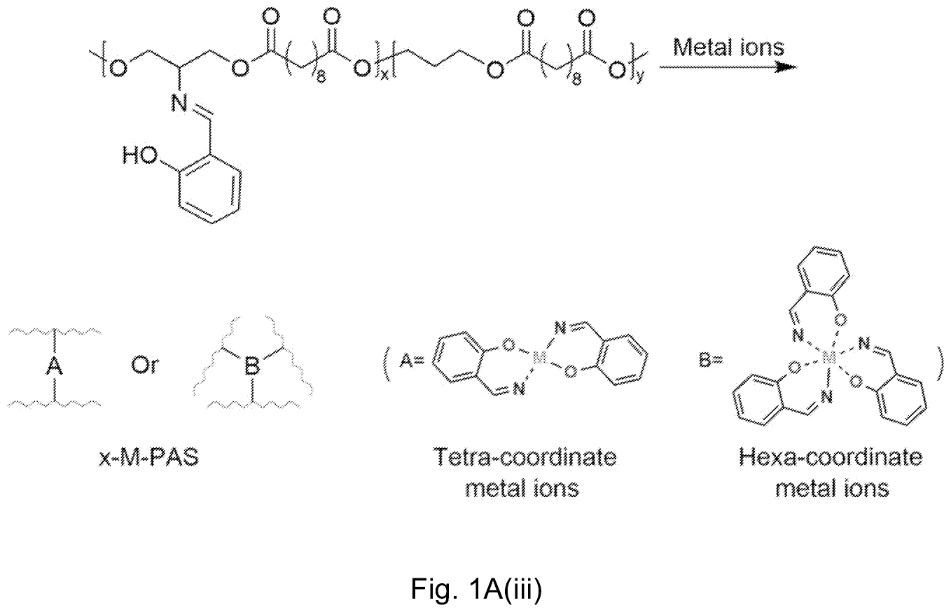

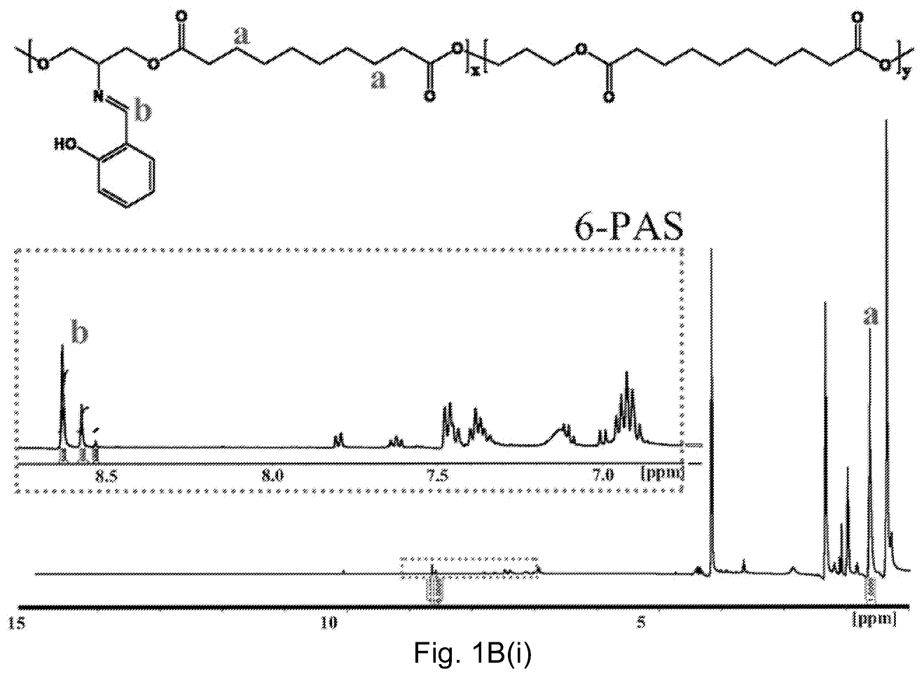

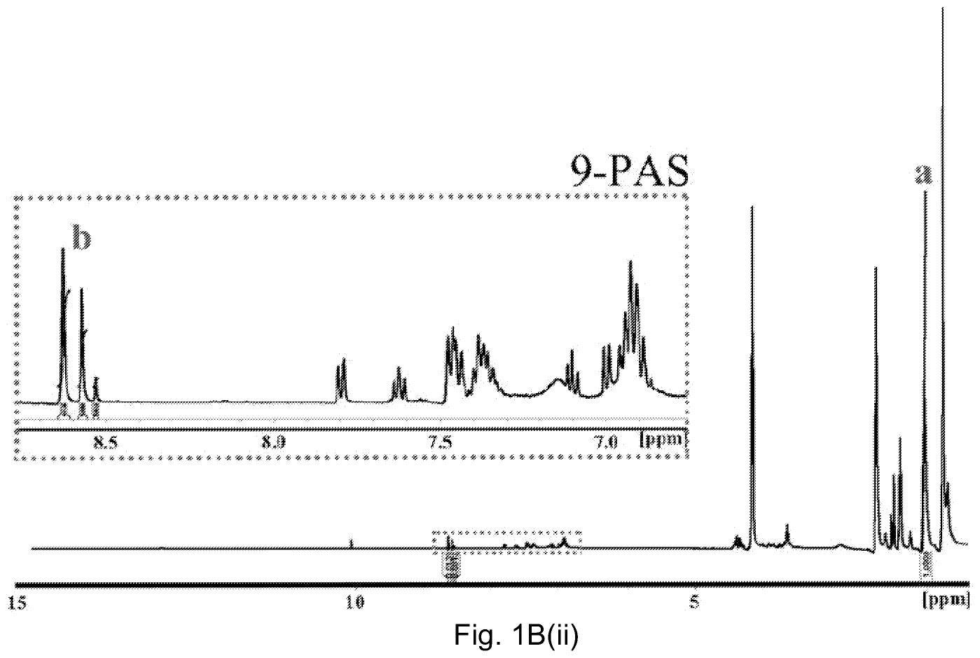

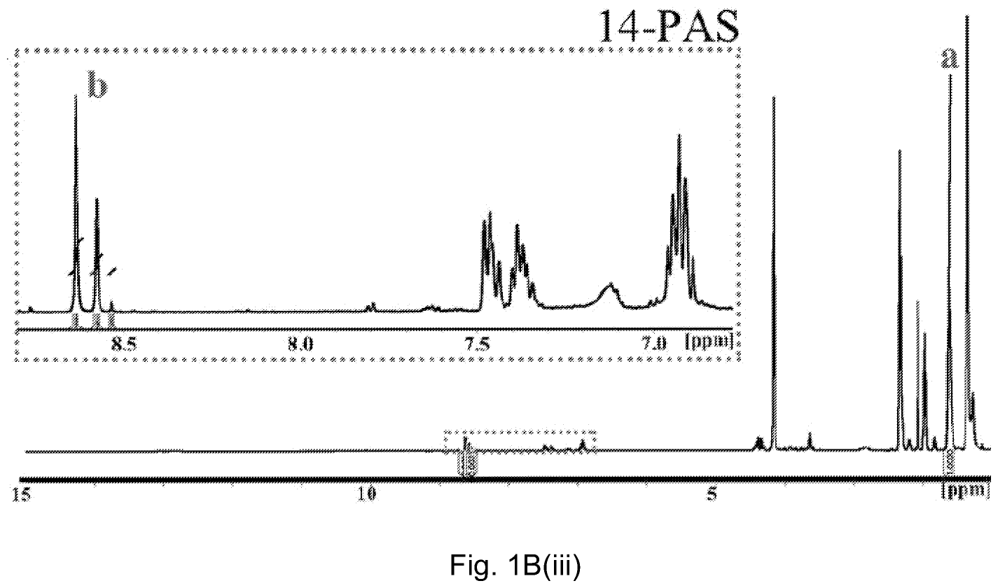

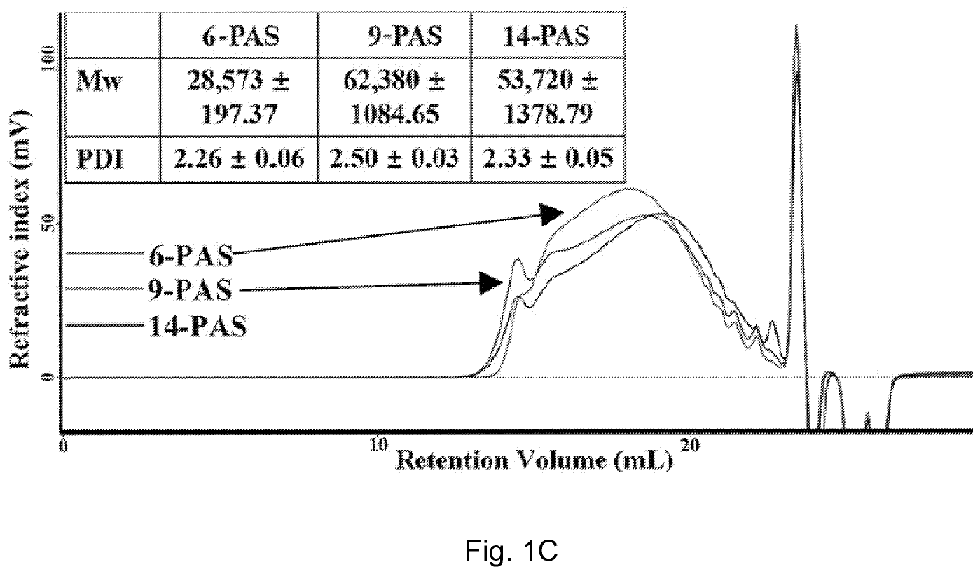

Monomer synthesis and structural analysis. The Schiff-base ligand 2-[[(2-hydroxyphenyl)methylene] amino]-1,3-propanediol (HPA, FIG. 1A(i)), bearing two hydroxyl groups and one phenol, was synthesized by a simple condensation.

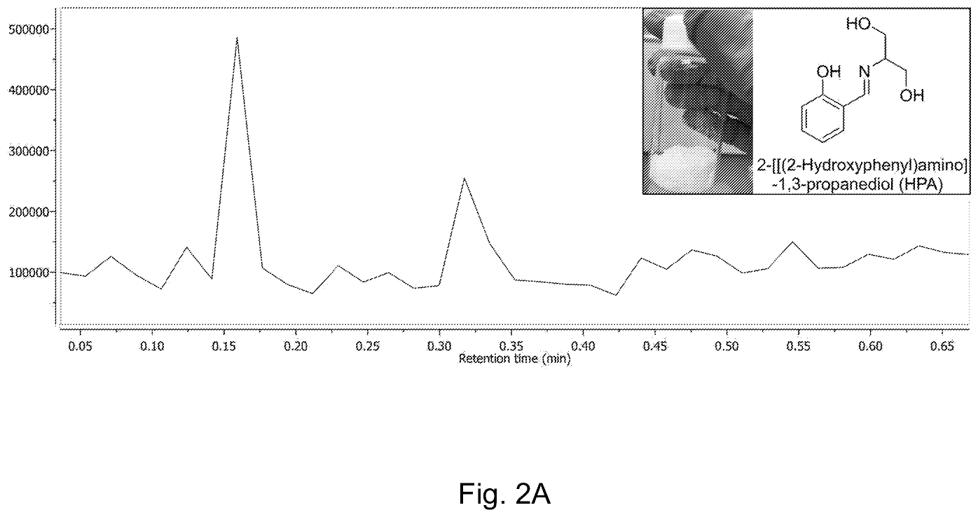

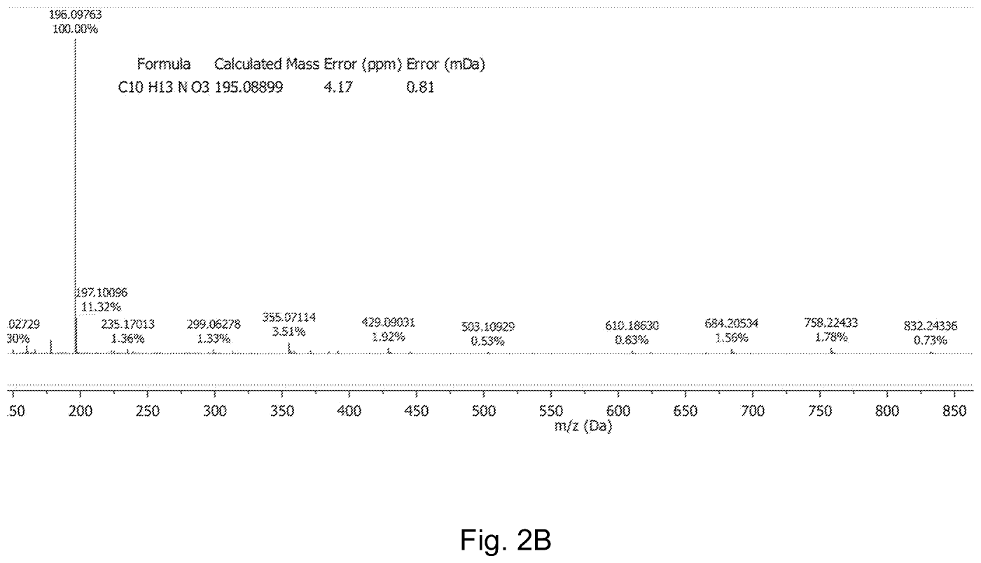

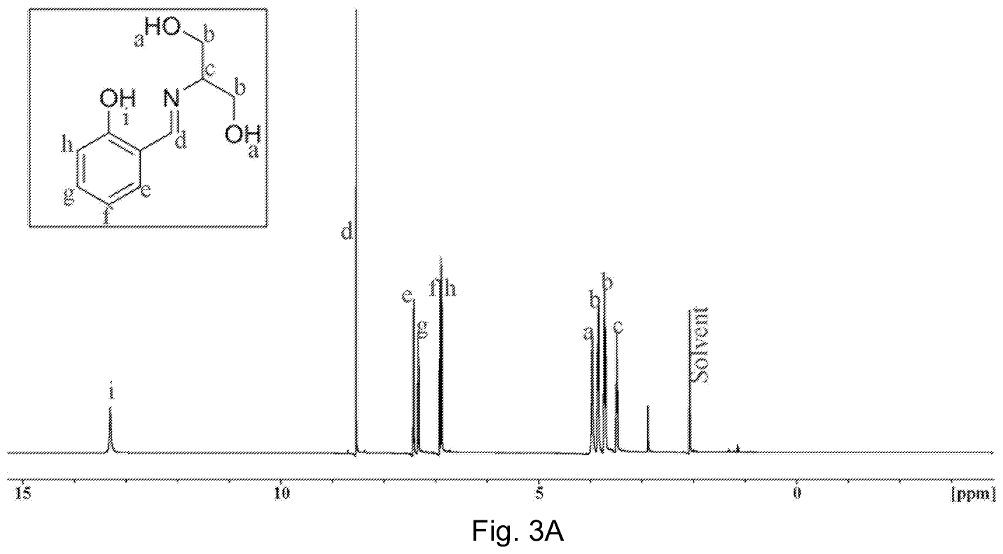

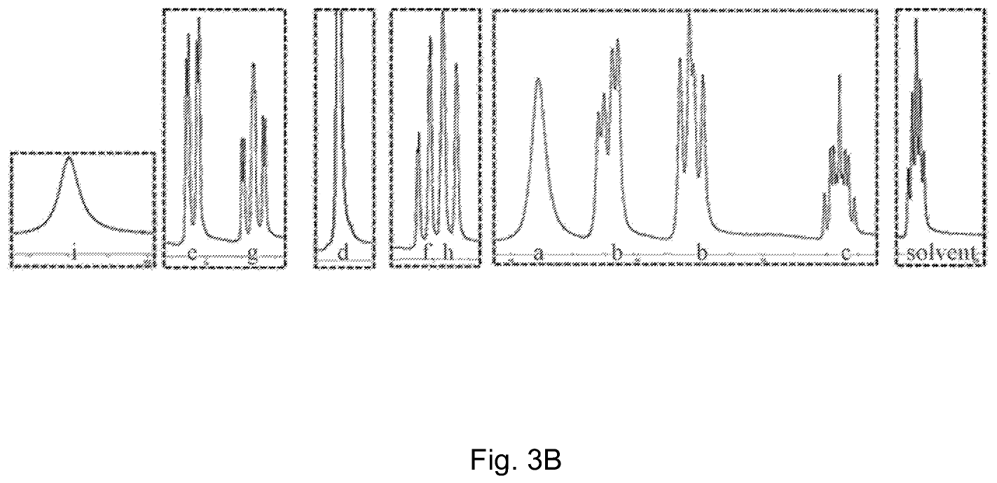

HPA was a bright yellow crystal with a formula of C₁₀H₁₃NO₃ and a molecular weight of 195.09 Da according to gas chromatography-mass spectrometry (FIGS. 2A-2B), matching the theoretical value (195.22). The proton nuclear magnetic resonance (¹H NMR) spectrum further identified the functional groups of HPA (FIGS. 3A-3B).

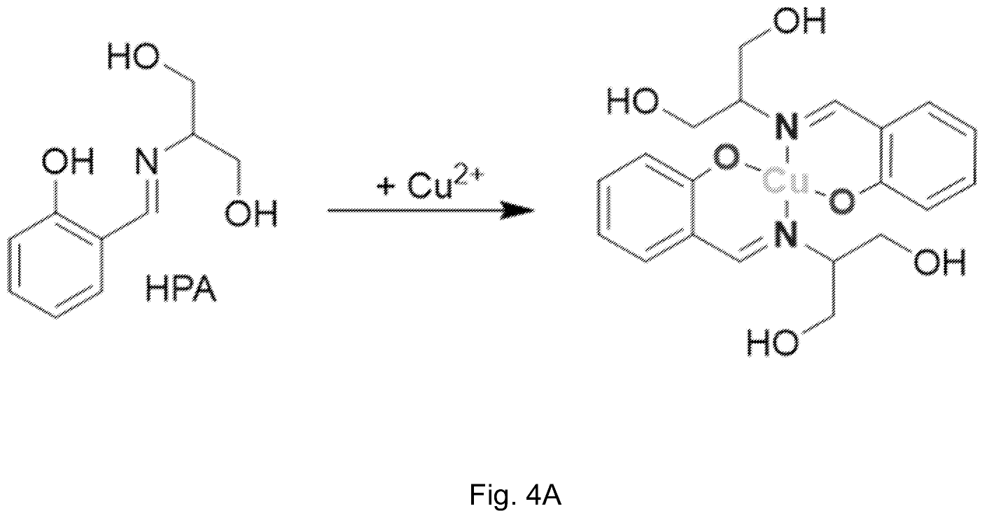

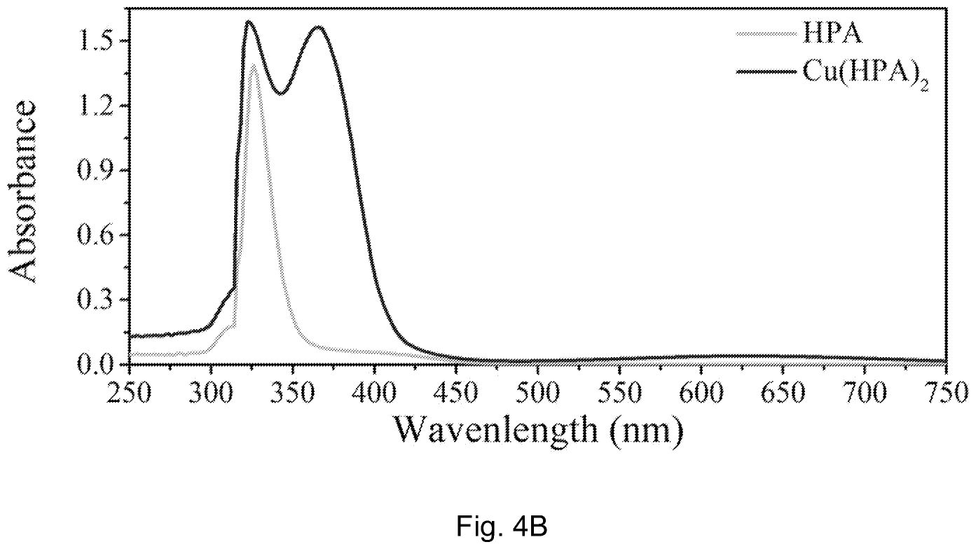

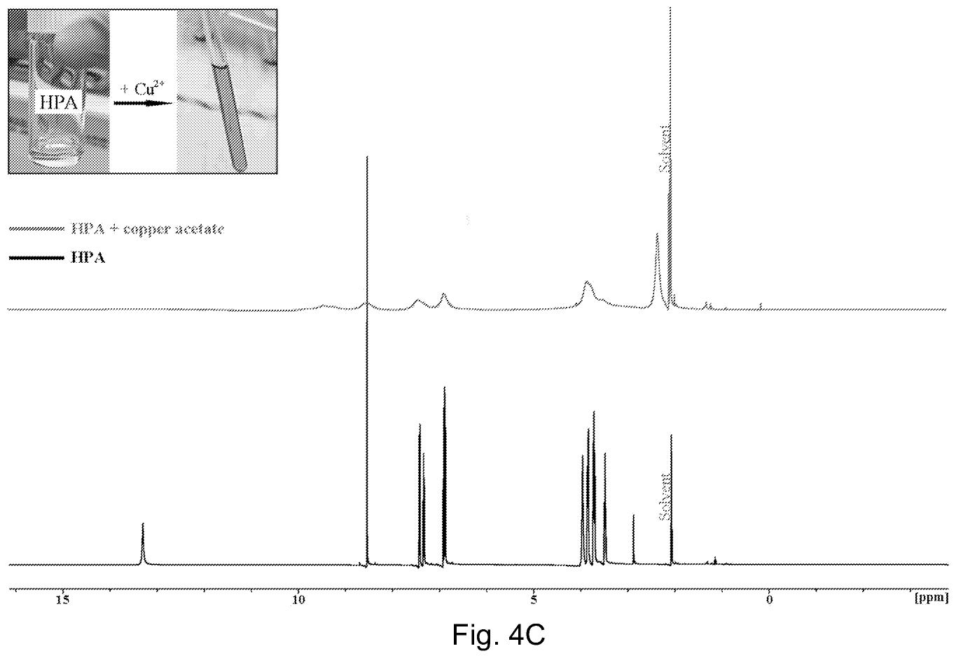

To examine HPA-metal chelation, a mixture of HPA and copper chloride at 2:1 molar ratio was analyzed by UV-visible spectrum (UV-vis) and ¹H NMR in acetone-d₆ (FIGS. 4A-4C). The HPA solution changed from yellow to green upon the addition of Cu²⁺. In the UV-Vis spectrum of HPA (FIG. 4B), the absorption band at 326 nm represented the π→π* transition. The formation of Cu(HPA)₂ shifted the π→π* transition from 326 nm to 322 nm. The band at 350-400 nm of Cu(HPA)₂ reflected the charge-transfer in the complex and the weak board band at 550-700 nm was due to d→d transition of the Cu²⁺ ion, both of which were absent in HPA. These electronic transitions corresponded to t_{2g}⁶e_{g}³ configurations for copper(II) ion in this complex, confirming the chelation of the ligand to the metal ions. In the ¹H NMR (FIG. 4C), all peaks in the Cu(HPA)₂ spectrum were shifted and broadened compared to that of HPA. The former results from the chelation between Cu²⁺ and the Schiff-base and the latter reflected the paramagnetism of Cu²⁺.

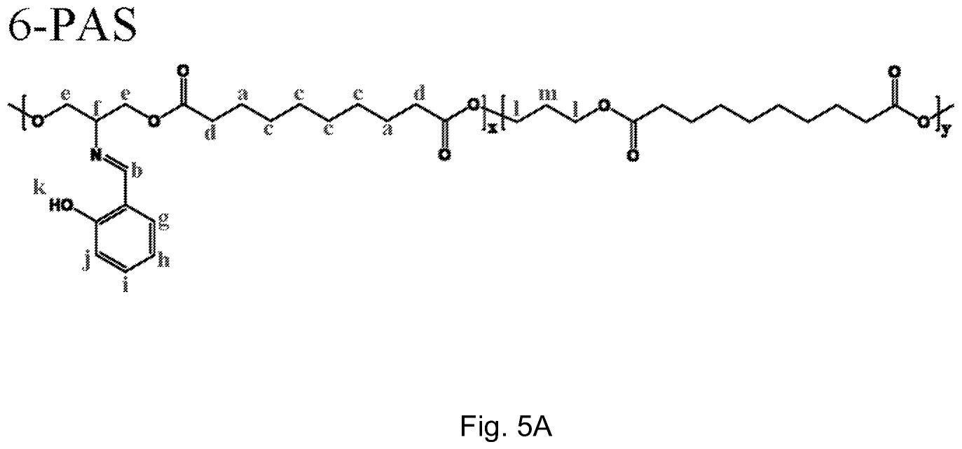

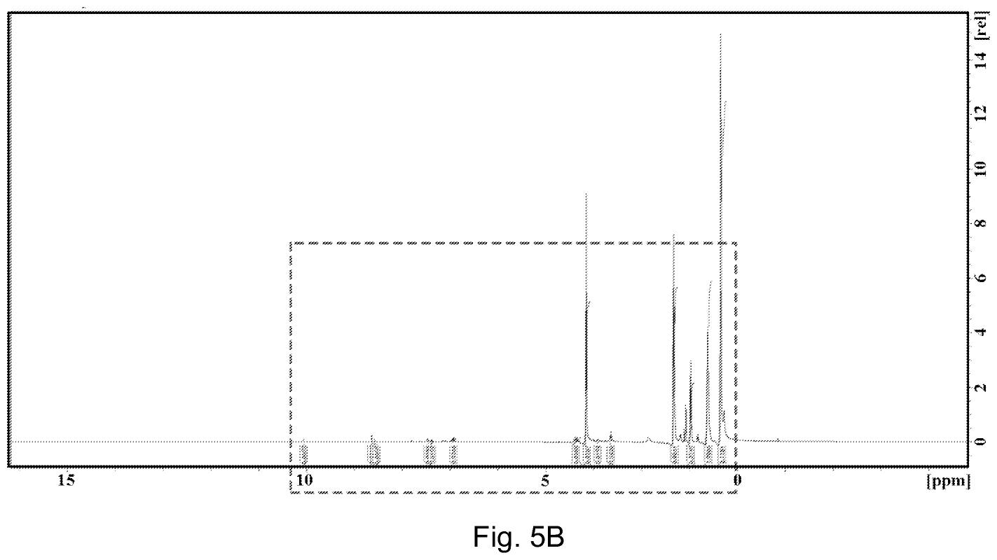

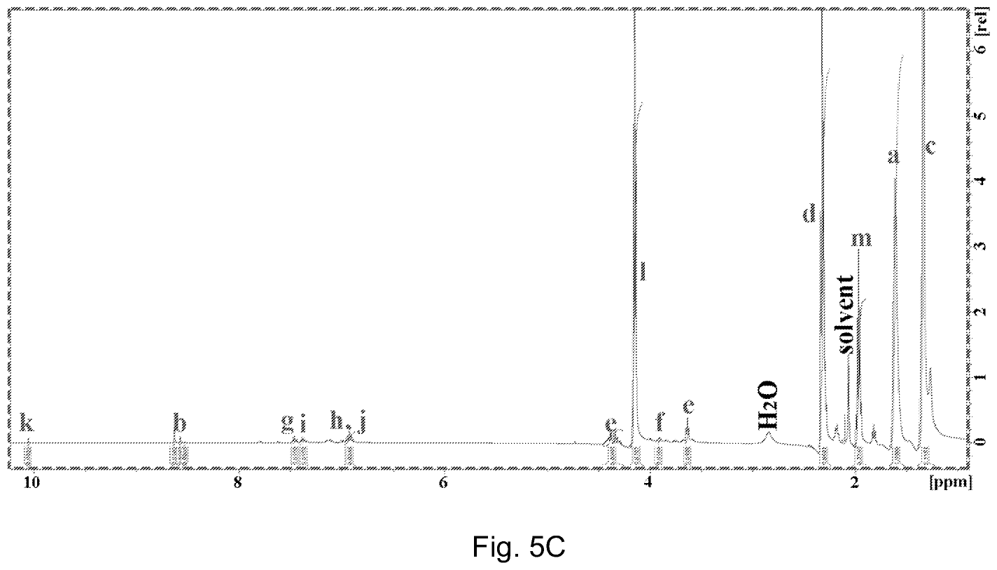

Polymer synthesis and structural analysis. After demonstrating the chelation capability of HPA, a series of biodegradable elastomers crosslinkable by metal ions were prepared. Polycondensation of 1, 3-propanediol, HPA and sebacic acid produced poly(propanediol-co-(hydroxyphenylmethylene)amino-propanediol sebacate) (PAS). The hydroxyl groups of 1, 3-propanediol, and HPA were converted into ester bonds of the polyester backbone, leaving the phenolic oxygen and Schiff-base as pendent groups (FIG. 1A(ii)). PAS polymers containing 3 different densities of ligands: 6%, 9% and 14%, were synthesized (denoted as 6-, 9- and 14-PAS, respectively) by adjusting the molar percentage of HPA in the diols (15%, 20% and 25% respectively, FIGS. 1B(i)-1B(iii)). The chemical composition of PAS was determined by ¹H NMR (FIGS. 5A-5C, 1B(i)-1B(iii)). The ligand density of PAS was quantified by the integral area of Schiff base proton H_{b} and sebacate methylene proton Hₐ using the following equations:

$$\frac{{H}_{b} \cdot x}{4 {H}_{a} ( x + y )} = \frac{{A}_{{H}_{b}}}{{A}_{{H}_{a}}} = \frac{0.0108 + 0.0042 + 0.0005}{1} , x = 6.2 \% \qquad ( 1 )$$

$$\frac{{H}_{b} \cdot x}{4 {H}_{a} ( x + y )} = \frac{{A}_{{H}_{b}}}{{A}_{{H}_{a}}} = \frac{0.0139 + 0.0083 + 0.0012}{1} , x = 9.36 \% \qquad ( 2 )$$

$$\frac{{H}_{b} \cdot x}{4 {H}_{a} ( x + y )} = \frac{{A}_{{H}_{b}}}{{A}_{{H}_{a}}} = \frac{0.0204 + 0.0111 + 0.0031}{1} , x = 13.84 \qquad ( 3 )$$

where A is the area, and x+y=1. Compared to the theoretical ligand density of 15, 20 or 25 mol %, the substitution rate of HPA in each reaction was approximately 41%, 47%, and 55% respectively. This relatively low degree of incorporation into the polymer was likely a result of the sublimation of HPA during the reaction, as yellow crystals were observed on the unheated part of the reaction flask. The Schiff base proton, H_{b}, split into 3 individual peaks (8.52-8.54, 8.56-8.59 and 8.61-8.65 ppm), reflecting the nature of different block sizes, neighboring groups and other fine structural differences of the block copolymer.

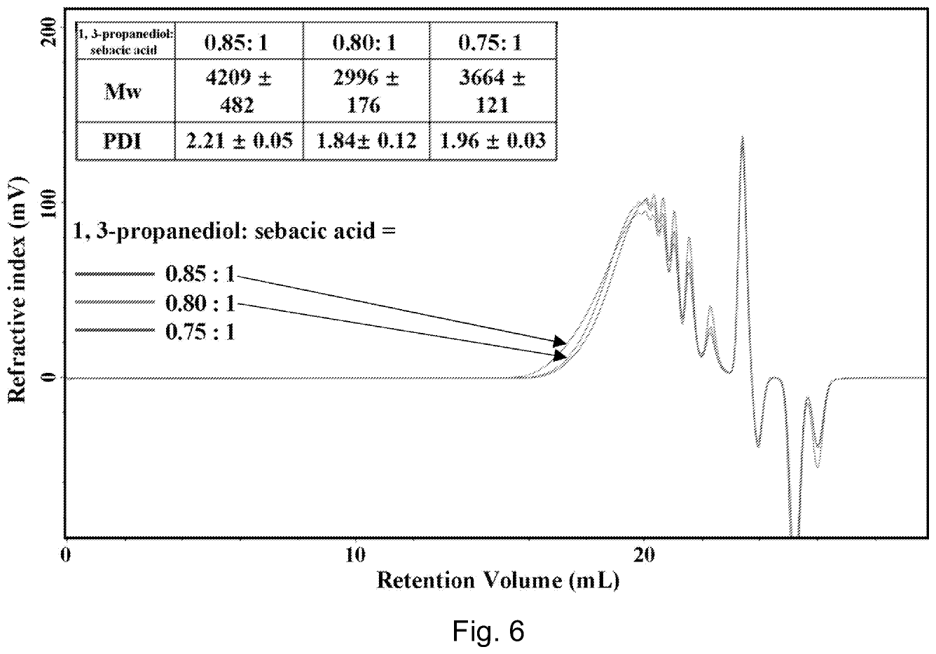

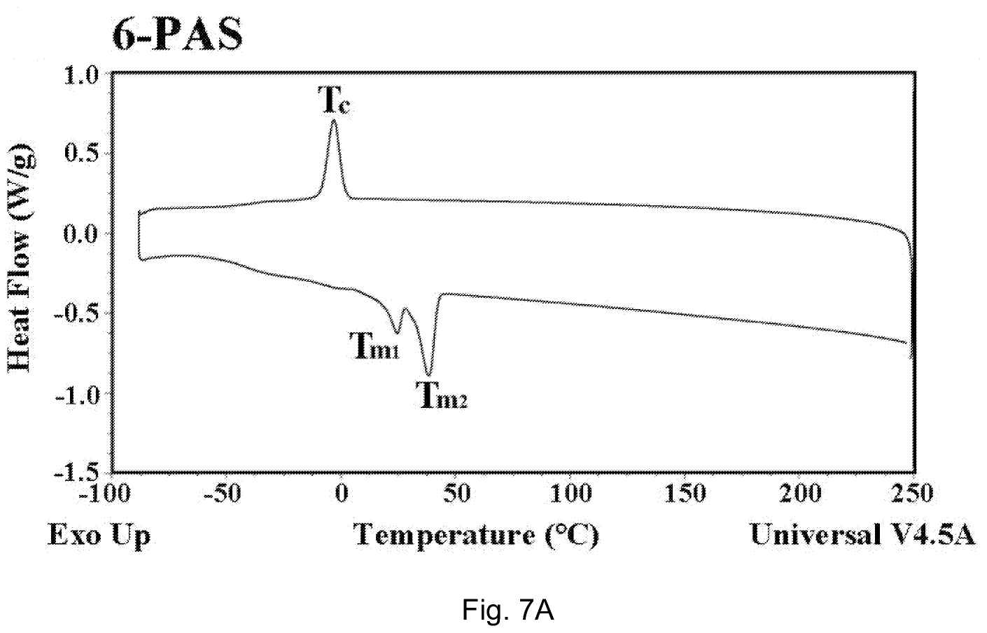

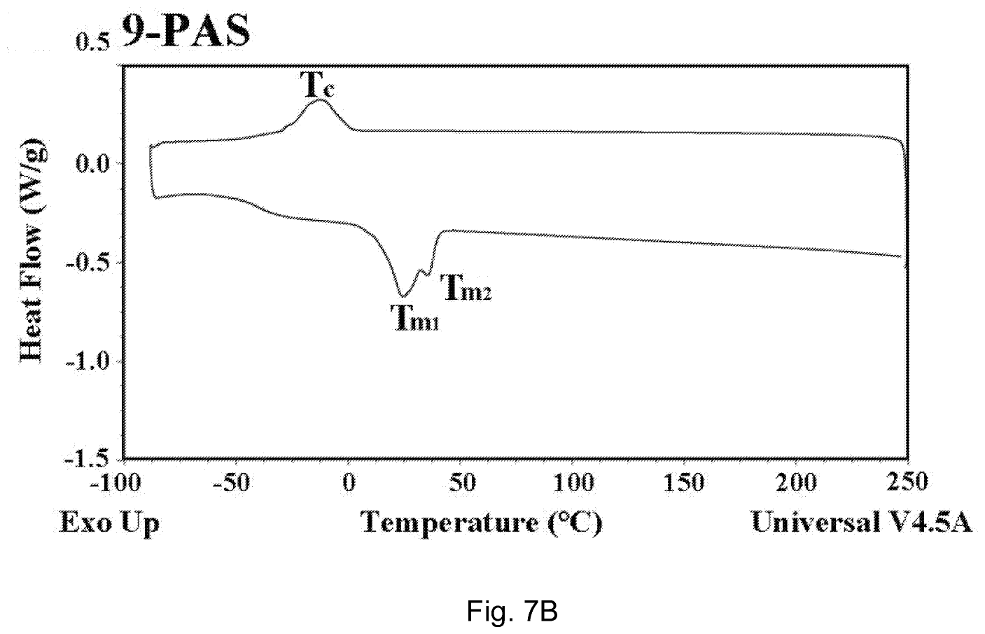

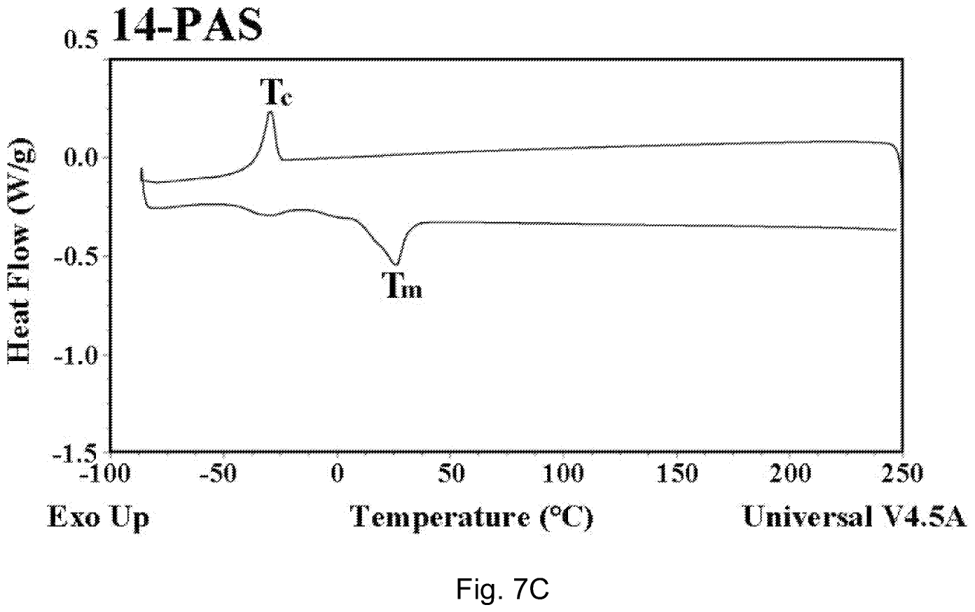

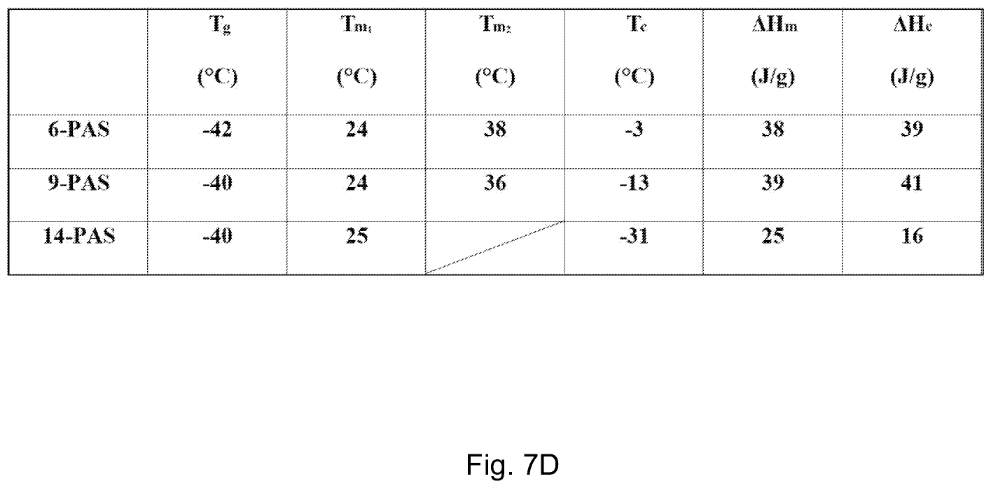

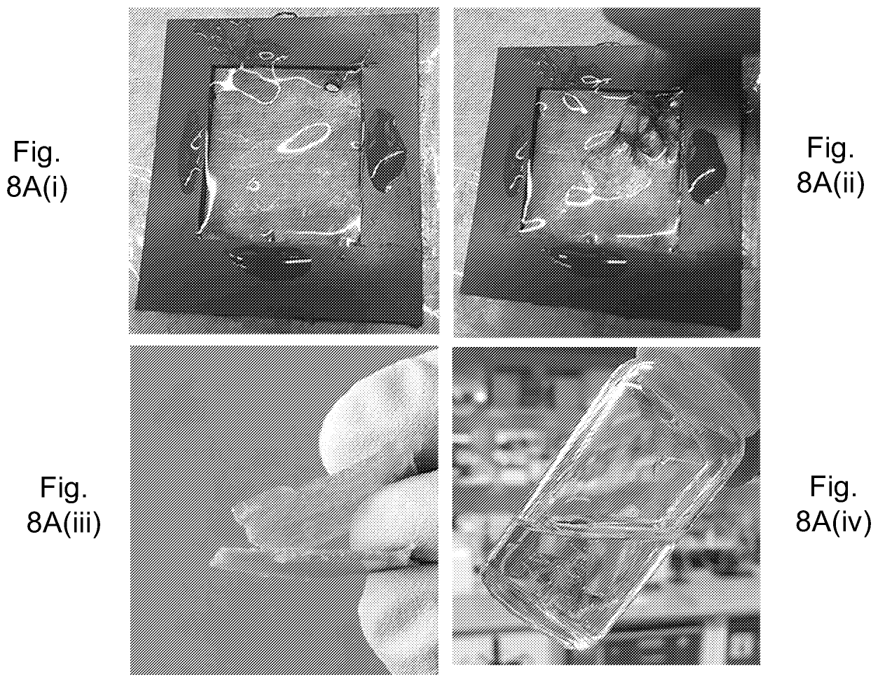

The intermediate product after steps 1) and 2) was an oligomer made from 1,3-propanediol and sebacic acid (FIG. 1A(ii)). The molecular weight of this intermediate was consistent among the 3 different types of PAS (FIG. 6). Reaction steps 3) and 4) with HPA resulted in 6-PAS, 9-PAS and 14-PAS with Mw of 28,573±197, 62,380±1085 and 53,720±1379 Da, respectively (FIG. 1C). The salicylaldimine side groups (ligand) were pendent on these polymer backbones, which affected the polymer chain packing, mobility, crystallization and consequently the thermal properties. All the PAS polymers demonstrated three thermal events: glass transition (T_{g}), melting (Tₘ) and crystallization (T_{c}) in differential scanning calorimetry (DSC), indicating they were semi-crystalline materials (FIGS. 7A-7D). As the ligand density elevated from 6 to 14%, T_{c} and the associated exothermic enthalpies (ΔH_{c}) reduced from −3 to −31° C. and 39 to 16 J/g respectively. Thus, the salicylaldimine pendants hindered the crystallization process. With the ligand density at 6%, 6-PAS showed two distinct melting temperatures at 24 and 38° C., indicating the presence of two types of crystalline structures likely due to different polymer chain length and relatively easy chain motion for crystallization. Notably, 14-PAS showed only one melting temperature at approximately 25° C. and a much larger difference between the exothermic enthalpies (ΔH_{c}) and the endothermic enthalpies (ΔHₘ) compared to the 6-PAS and 9-PAS. This further supported the notion that the pendent ligands hindered polymer packing into crystalline domains. On the other hand, the T_{g} reflected the chain flexibility of the PAS polymers. It was noteworthy that the T_{g} increased from −42° C. for the 6-PAS to −40° C. for both the 9-PAS and 14-PAS. Although the T_{g} changes were moderate against the rising ligand density, the glass transition became more distinct in the DSC curves, indicating more amorphous structures were arrested in the polymers as the ligand content increased. This was consistent with ligands interfering with crystalline packing and consequently increasing the amorphousness of the polymer.

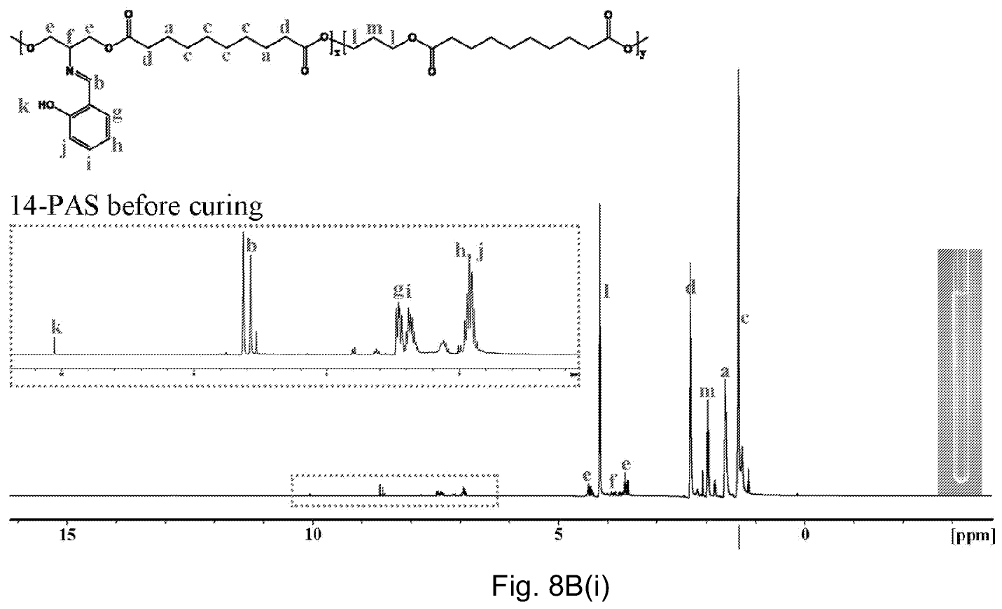

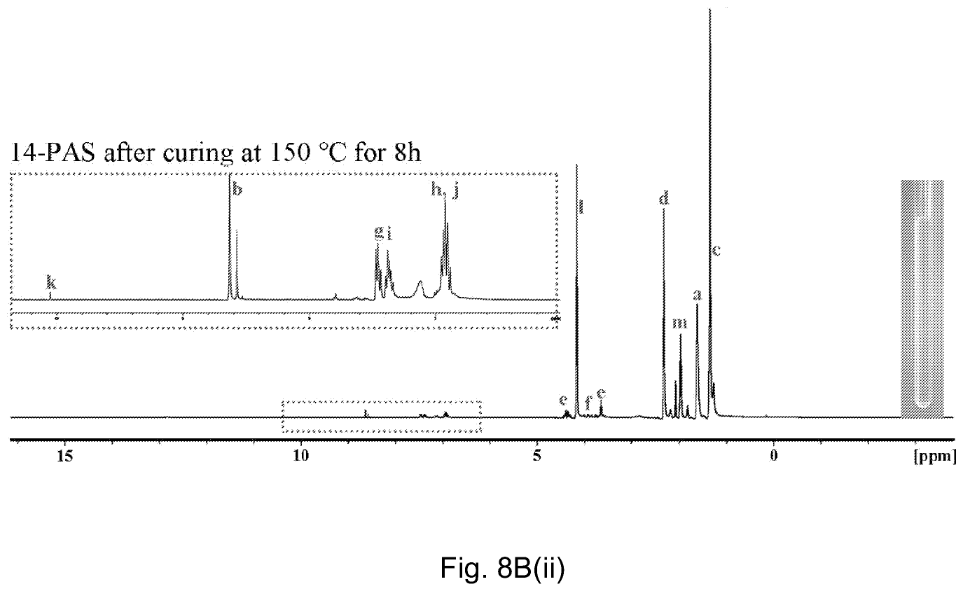

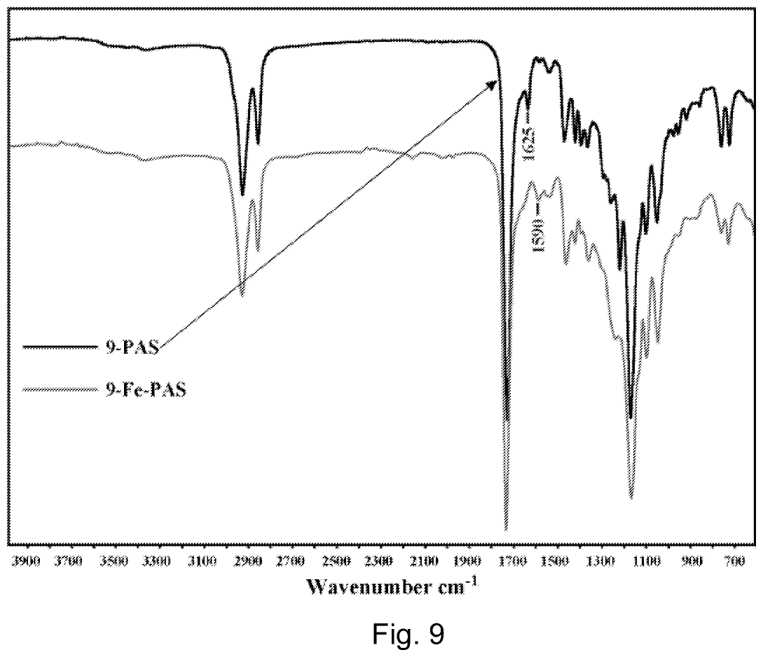

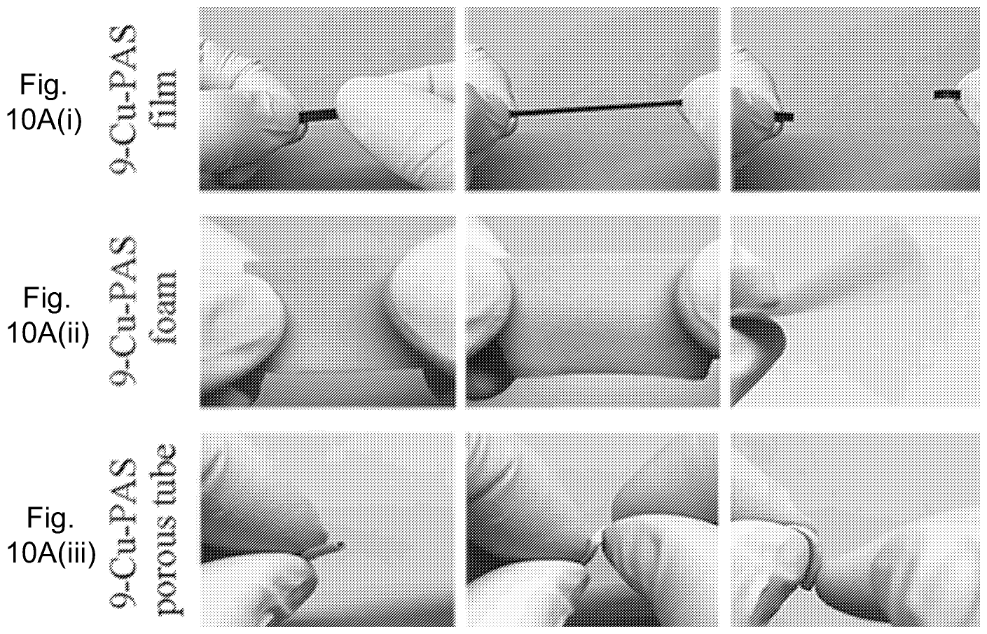

Metal ions-mediated crosslinking. Metal coordination bonds are found in natural and synthetic materials. The coordination bonds imparted versatility to the polymer network because a ligand designed to bind different types of metal ions provided additional control to polymer properties such as stiffness, toughness and viscoelastic dissipation. Here, two or three of the ligands formed tetra- and hexa-coordinate chelates with metal ions leading to crosslinking of the polymer chains. Focus was placed on biologically relevant metal ions: Mg²⁺, Ca²⁺, Fe³⁺, Co²⁺, Cu²⁺ and Zn²⁺. Immediately after the addition of a metal ion, for example, Fe³⁺, the PAS solution gelled, indicating crosslinking. Chelation bonds are reversible at the presence a competing ligand. The gel returned to a solution within 1 minute of adding EDTA, an excellent ligand to many transition metals. Polymers are large molecules, with lower mobility and solvent diffusivity than small molecules. Therefore, after the mixing of the polymers and metal ions in solution, the crosslinked polymers (M-PAS) were heated at 150° C. for 8 hours at 30 mTorr to remove solvents. To test if heating introduced additional crosslinks, PAS alone was subjected to identical heating protocols. PAS stayed stable when heated to 250° C. with no noticeable decomposition, as revealed by DSC (FIGS. 7A-7D). PAS heated at 150° C. and 30 mTorr for 8 hours dissolved well in organic solvents such as acetone (FIG. 8A(iv)), unlike the crosslinked M-PAS. 1H NMR spectra of 14-PAS before and after heat treatment were virtually identical (FIG. 8B). Thus, heating alone caused no appreciable crosslinking. In the Fourier-transformed infrared (FTIR) spectrum of 9-Fe-PAS, the shift of ν(C═N) mode from 1625 cm⁻¹ in the free ligand to 1590 cm-1 indicated bond formation through imino nitrogen atom, further corroborating chelation between salicylaldimine of PAS and iron (FIG. 9).

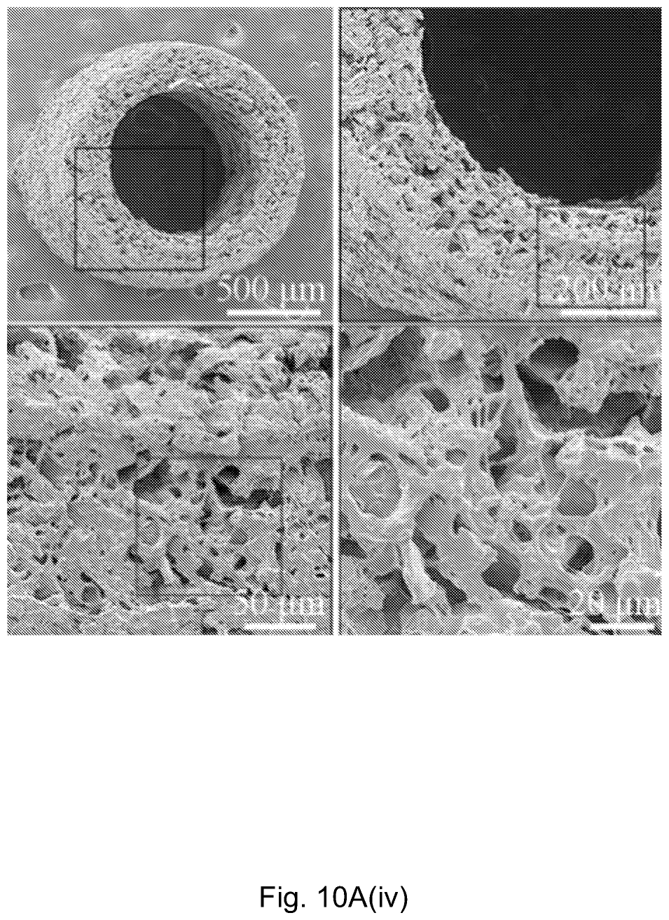

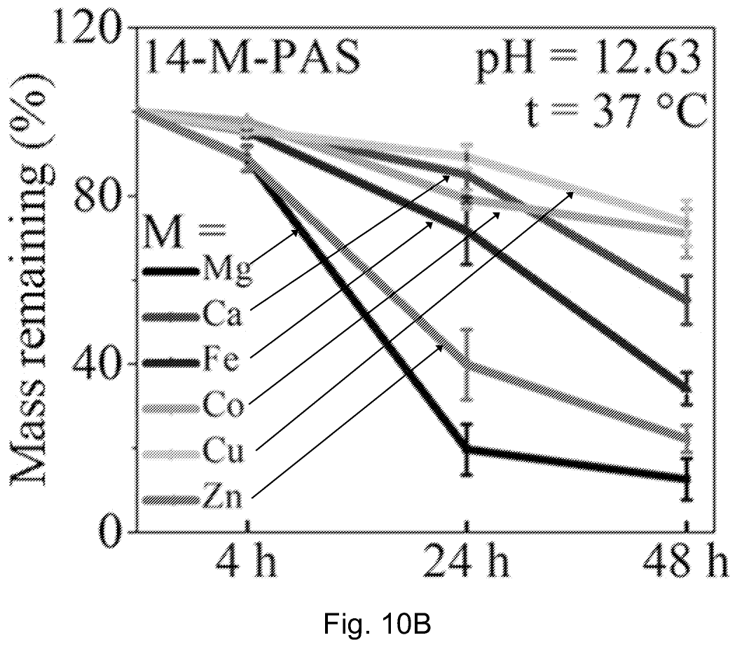

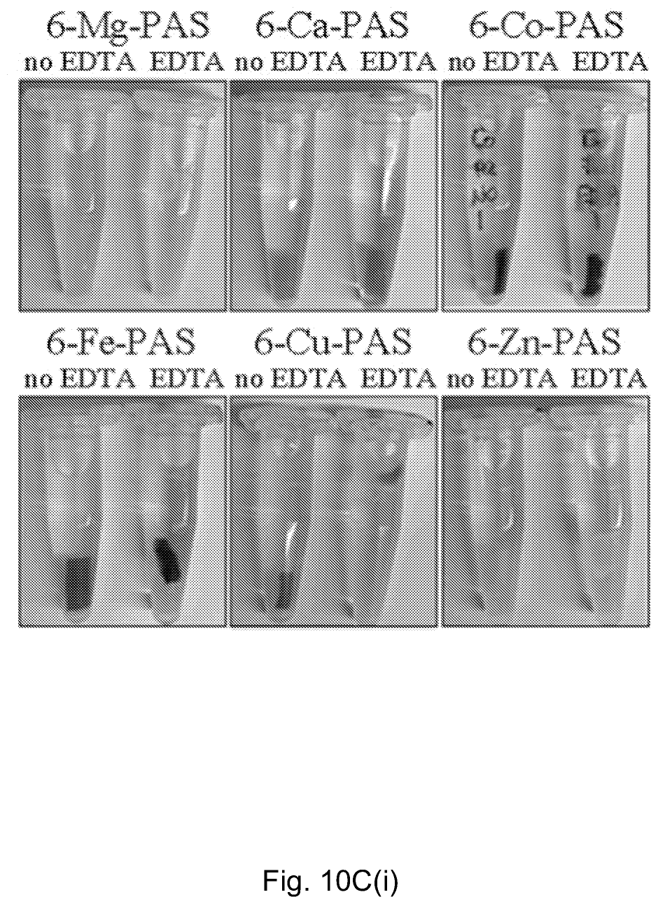

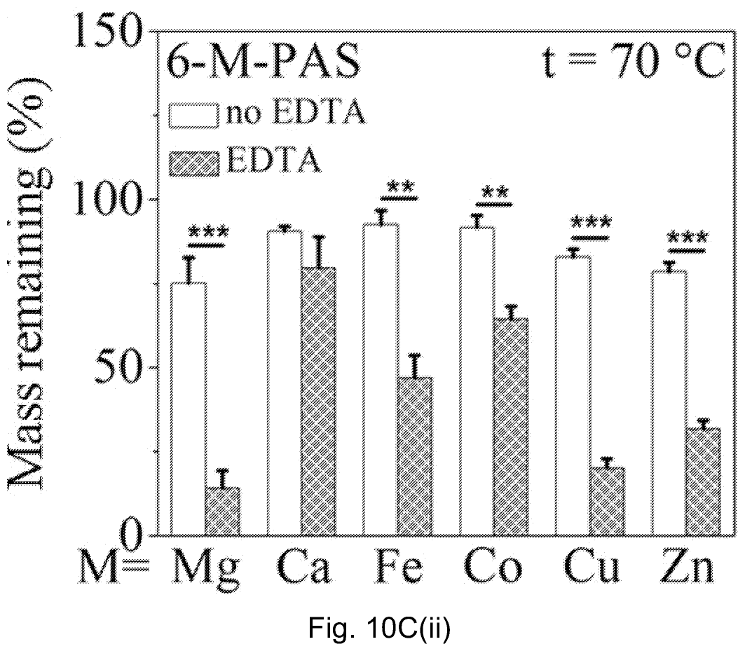

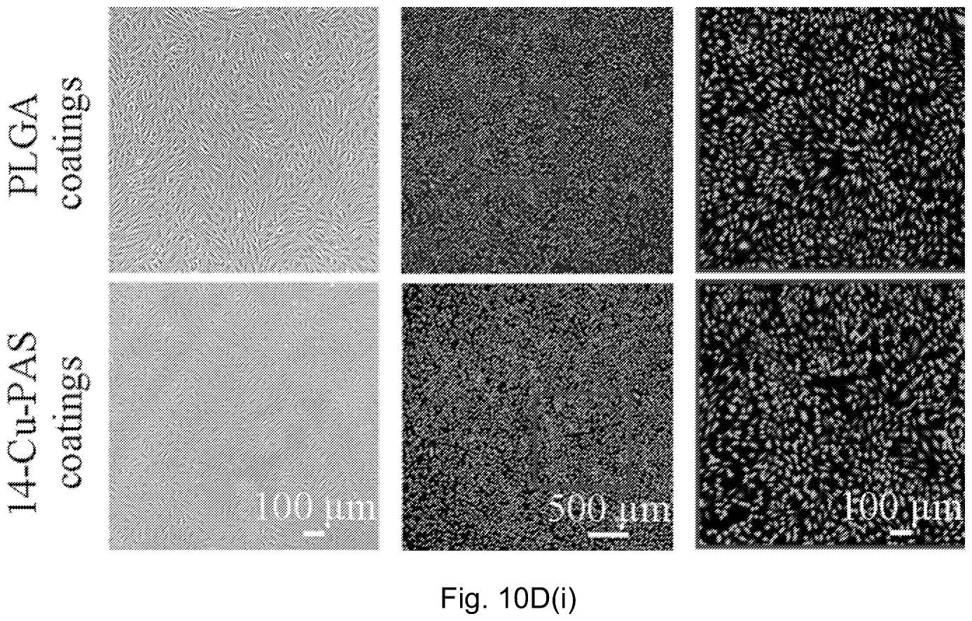

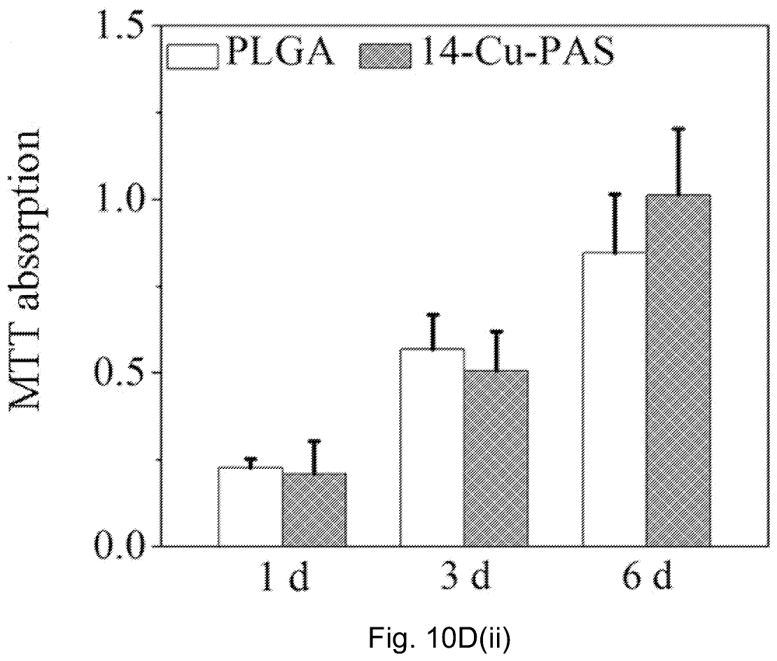

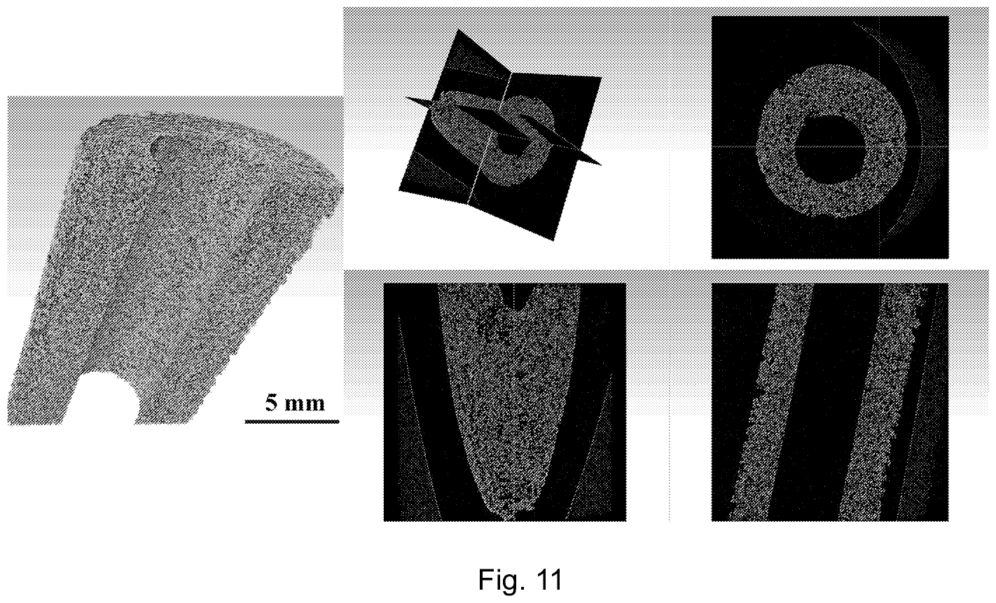

Processing of M-PAS into various forms. As indicated by the DSC results, PAS was amorphous at 37° C. The uncrosslinked PAS polymer was processed into various shapes, because it melted into a liquid and dissolved in common organic solvents such as acetone, anisole, tetrahydrofuran, isopropanol, chloroform, dimethylsulfoxide, N, N-dimethylformamide and hexafluoroisopropanol. M-PAS was prepared into films, foams and porous tubes (FIGS. 10A(i)-10A(iv)). Casting the mixtures of an acetone solution of PAS and metal ions into silicone molds and heating at 150° C. and 30 mTorr for 8 hours produced M-PAS elastomer films. The M-PAS elastomer foams were prepared under the same conditions except that NaCl particulate (32˜53 μm) was added as porogens. The M-PAS elastomer porous tubes were prepared with NaCl particulate, a stainless steel rod as a mandrel, and a Teflon® tube as the outer mold and under the same heat treatment. Using 9-Cu-PAS elastomer as an example, the films and foams exhibited excellent elastic recoil (FIGS. 10A(i)-10A(iii)). The 9-Cu-PAS elastomer porous tube had a porosity of approximately 65% (Micro-CT, FIG. 11) and was twisted repeatedly without deformation (FIG. 10A(iii)).

Degradation of M-PAS. The ester backbone of an M-PAS elastomer made it degradable by hydrolysis. An accelerated in vitro degradation in a PBS solution containing 60 mM NaOH (pH=12.63) was used to investigate the degradation of 14-M-PAS elastomer films. After 48 hours under agitation at 37° C., the 14-M-PAS elastomer films crosslinked by Mg²⁺, Ca²⁺, Fe³⁺, Co²⁺, Cu²⁺ and Zn²⁺ degraded gradually and the mass remaining (%) was 12.63±4.91%, 55.22±5.76%, 34.13±3.87%, 71.03±5.74%, 73.53±5.44% and 22.13±3.21%, respectively (FIG. 10B). The degradation rate of M-PAS elastomers varied with metal ions, which might have been related to their different metal-ligand chelating strength. The most durable among these, 14-Cu-PAS elastomer, hydrolyzed 5.83 times more slowly than the fastest variant crosslinked by Mg²⁺.

Covalent crosslinking is irreversible. Weak bonds including hydrogen bonds, π-stacking, polar interactions and hydrophobic interactions are reversible. Coordination bonds are reversible in that another chelator can compete for the metal ions and break the bond. A 10 mM EDTA solution in DMF/H₂O (1/1, v/v) extracted metal ions from the 6-M-PAS elastomers within 72 hours under agitation at 70° C., accompanied by appreciable polymer degradation (FIG. 10C(i)). After EDTA extraction, the mass remaining (%) for 6-M-PAS elastomer films ranged from 14.08±5.24% to 79.61±9.26%, depending on the metal ions (FIG. 10C(ii)). This suggested that different strength of the chelation bonds determined the stability of the crosslinked polymer. The faster degradation of 6-M-PAS elastomer in the presence of EDTA demonstrated that metal coordination bonds were holding the polymer network together and that the crosslinking was reversed by another ligand. This offers a new way to control polymer degradation.

Cytocompatibility of M-PAS. The cytocompatibility was examined by culturing human umbilical vein endothelial cells (HUVECs) on coatings of the elastomers. The commercially sourced poly(D, L-lactide-co-glycolide) (PLGA) served as a control. Cu²⁺ was chosen because among the ions used, Cu²⁺ is a heavy metal and can damage the cells. HUVECs maintained typical endothelial morphology and displayed the same proliferating and spreading behavior on both 14-Cu-PAS elastomer and PLGA coatings with few dead cells (FIGS. 10D(i)-10D(ii)). MTT assay demonstrated effectively the same metabolic activity on 14-Cu-PAS elastomer and PLGA. These results suggested that 14-Cu-PAS elastomer was at least as biocompatible as PLGA in vitro. The good cytocompatibility of 14-Cu-PAS elastomer reflected the fact that the copper content was low and the starting materials for PAS: HPA, sebacic acid and 1,3-propanediol were non-toxic.

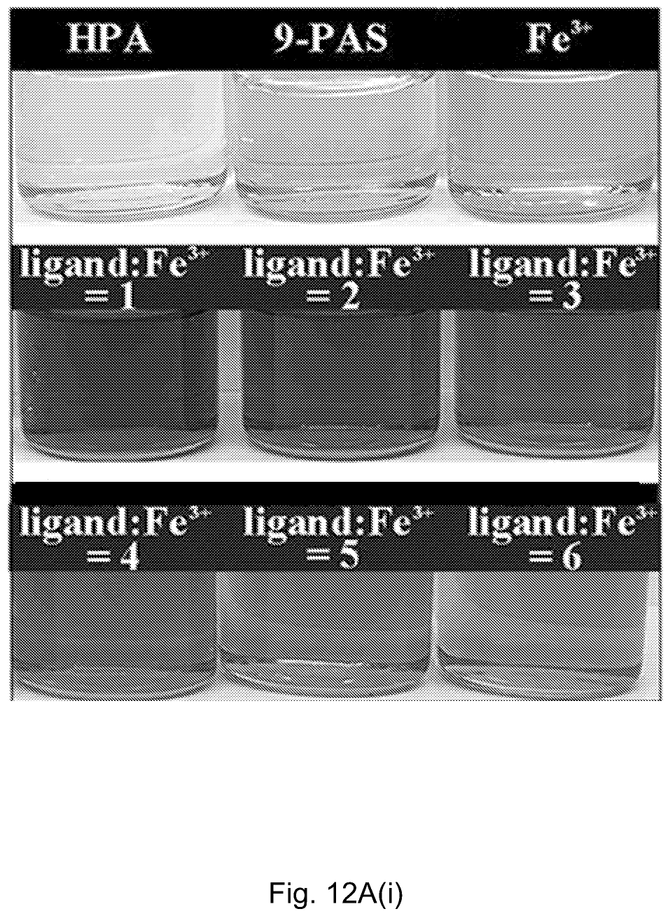

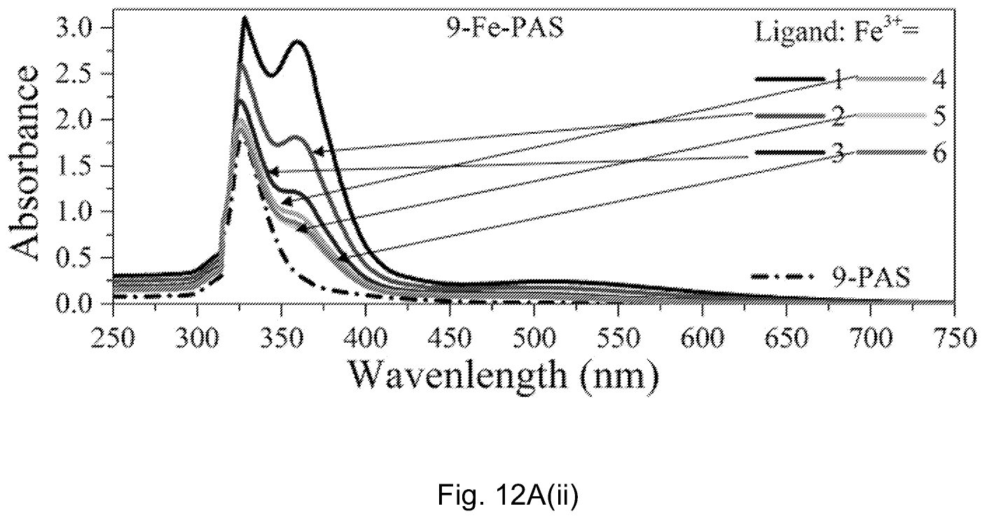

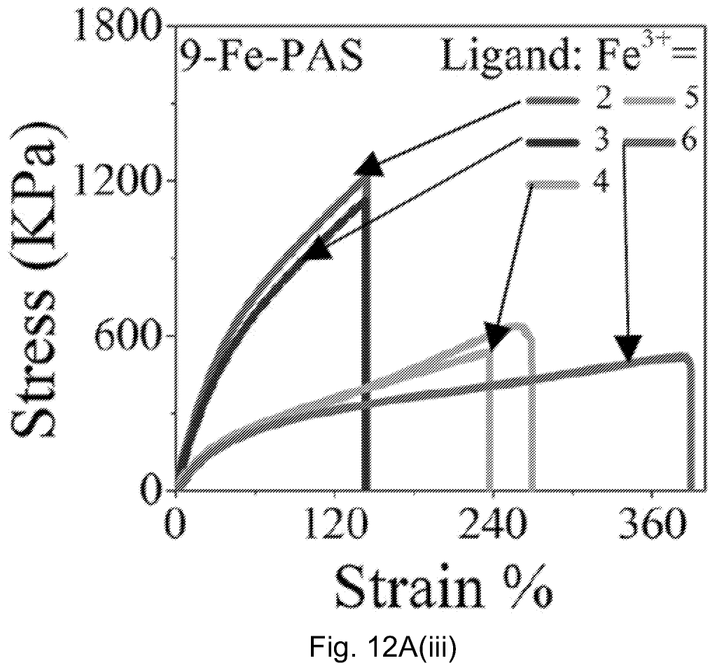

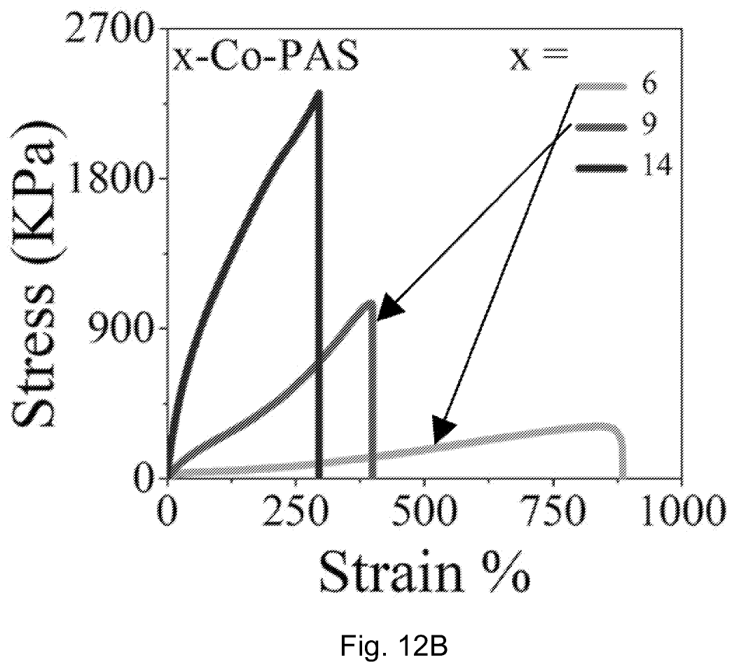

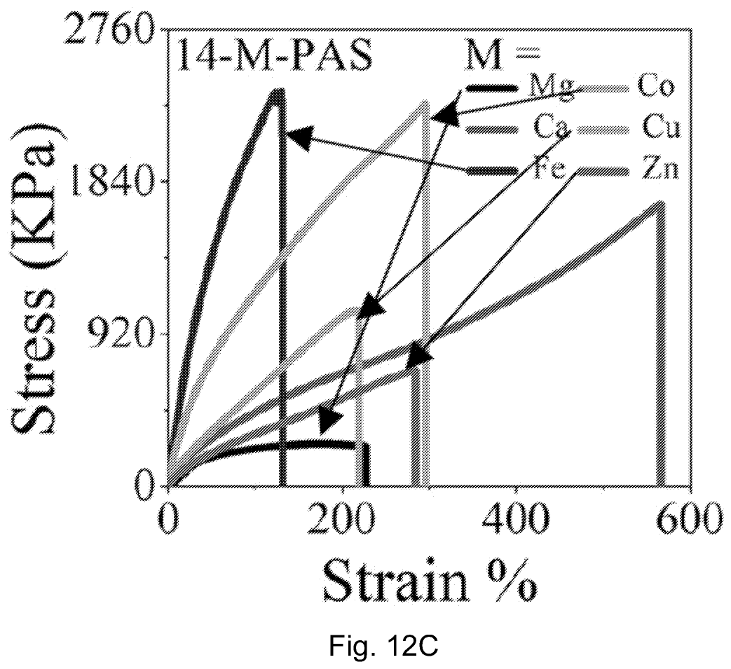

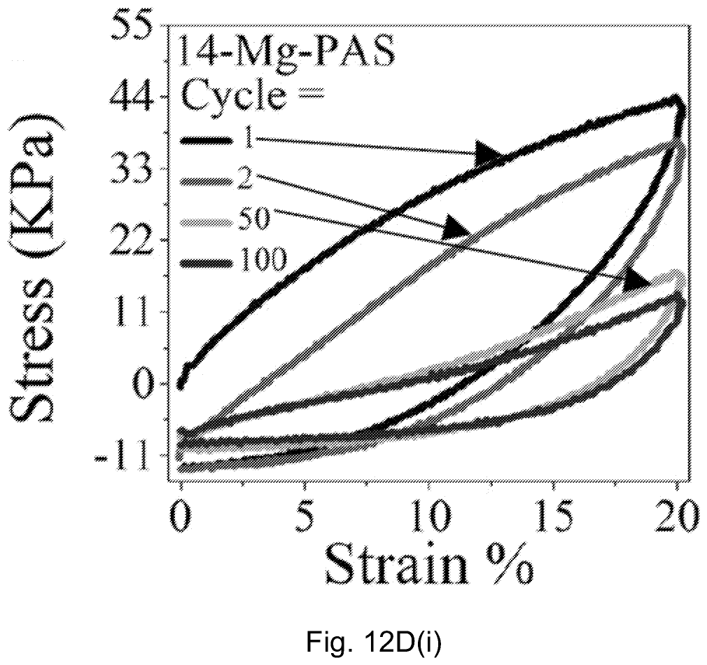

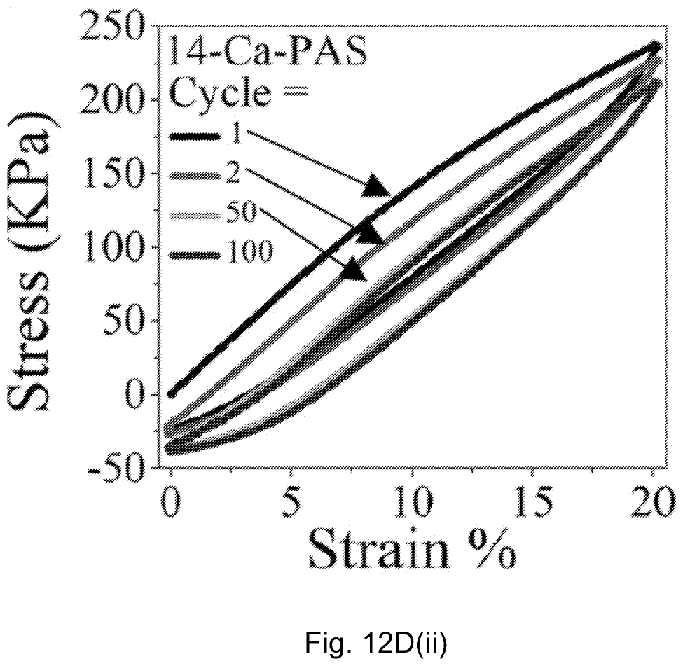

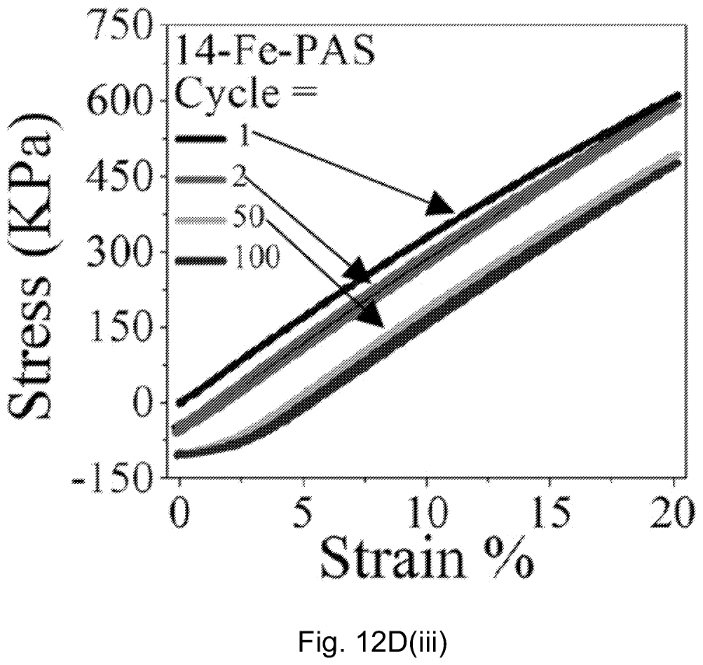

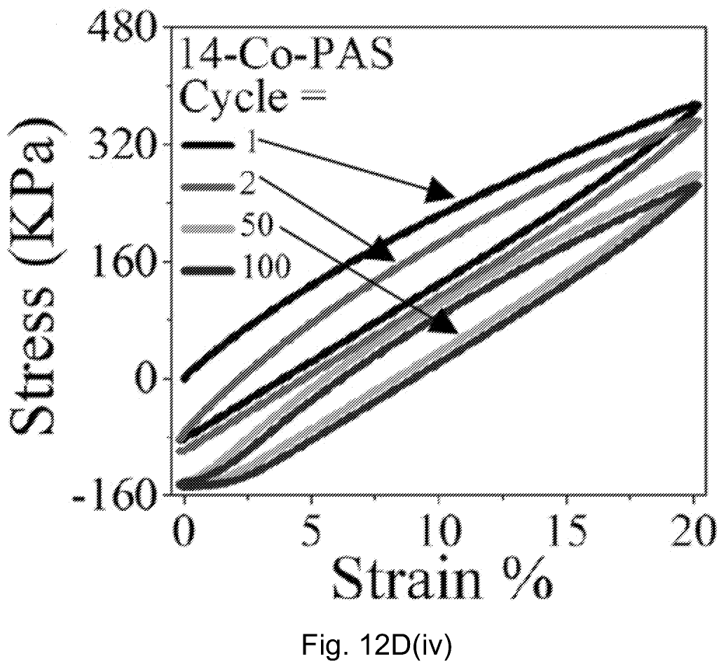

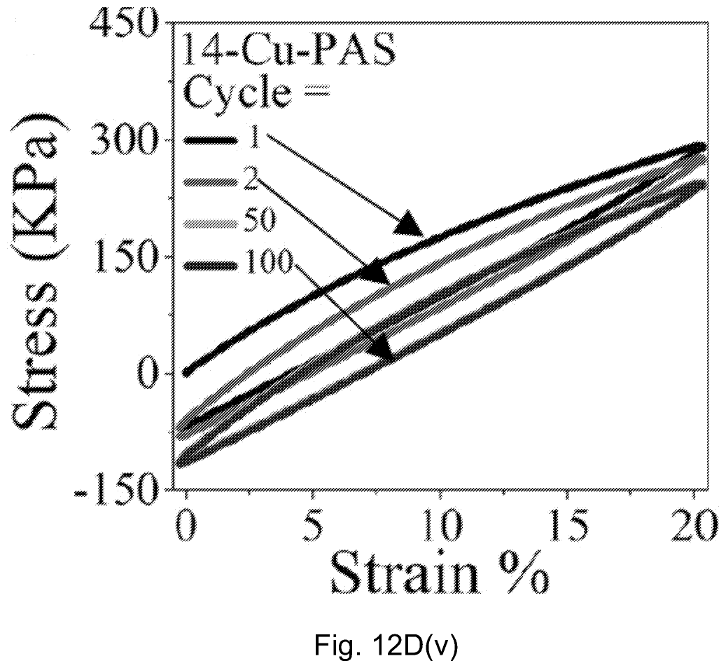

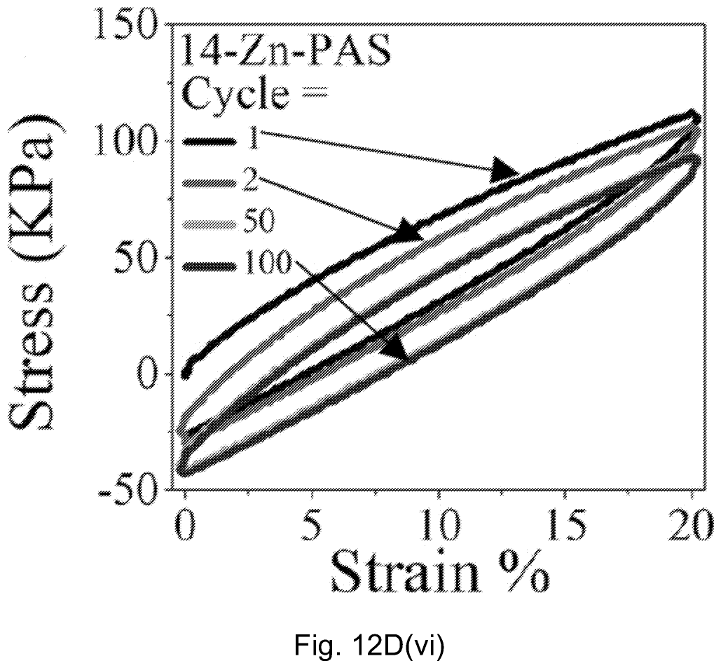

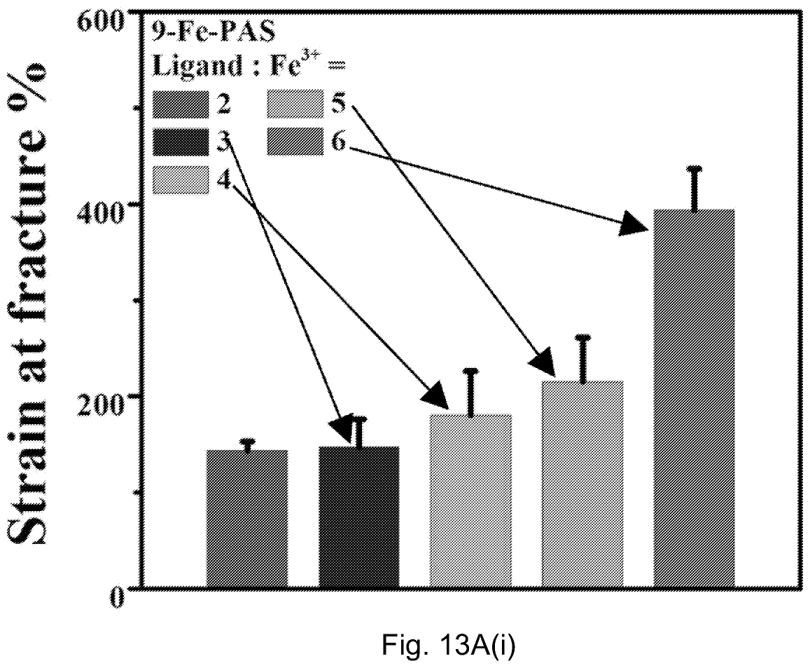

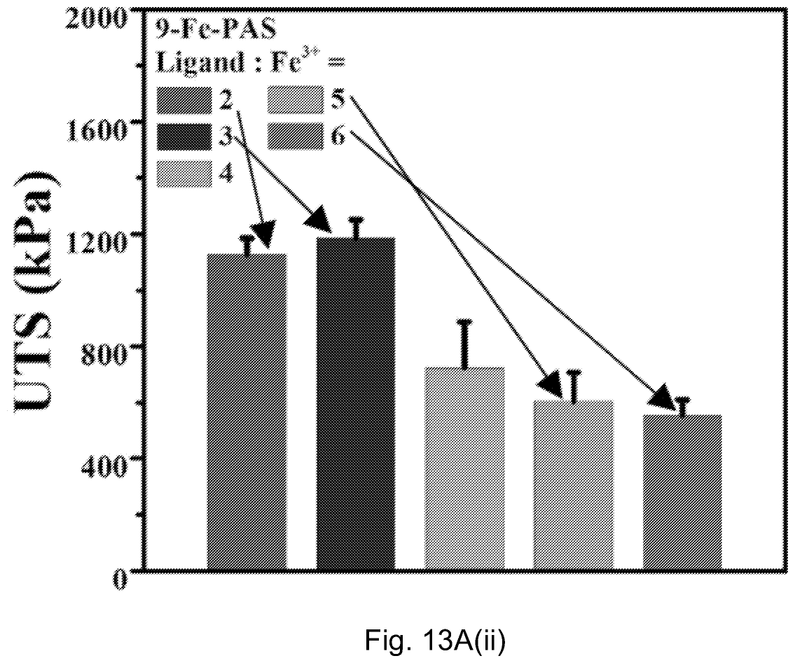

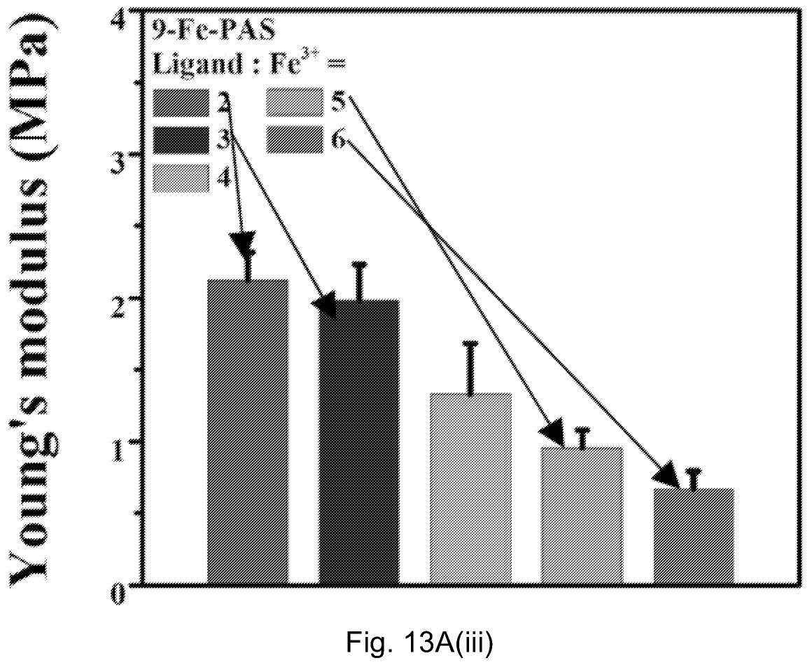

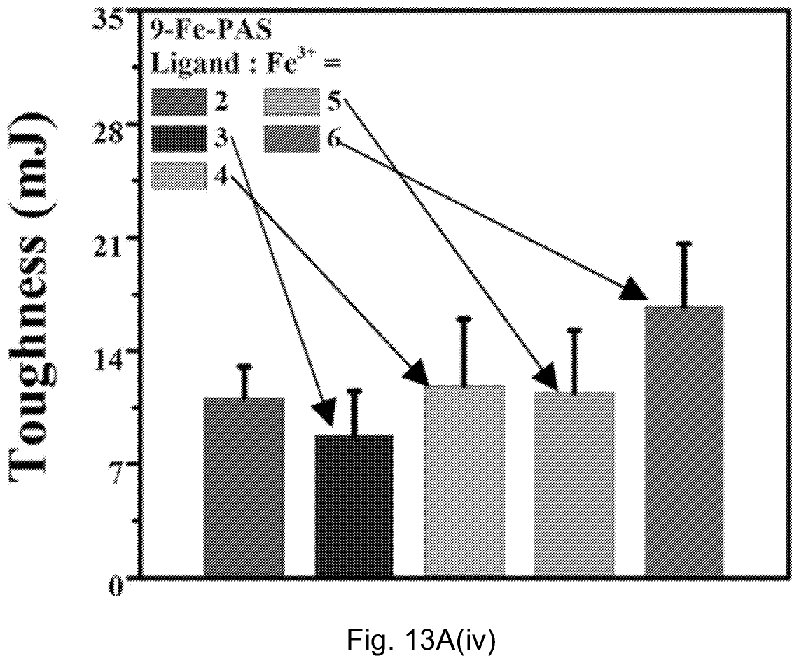

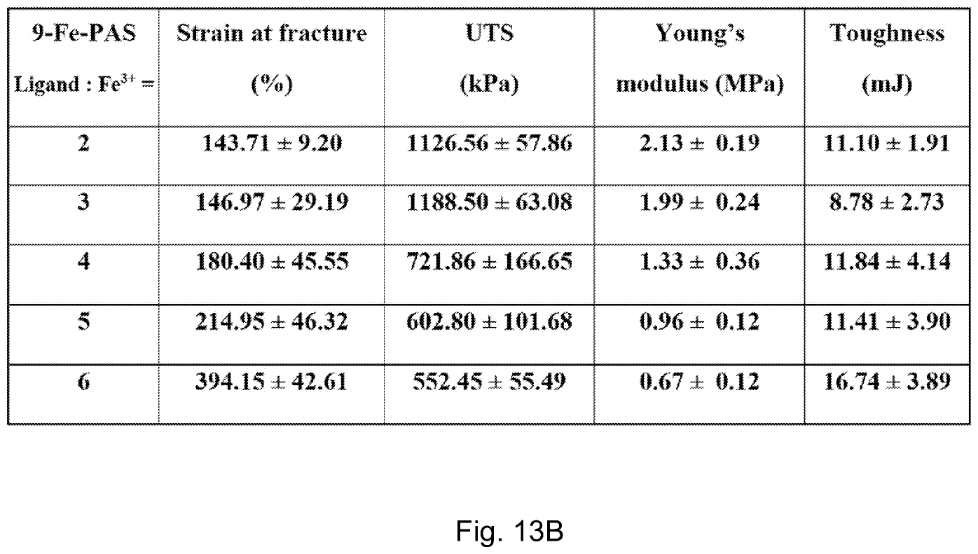

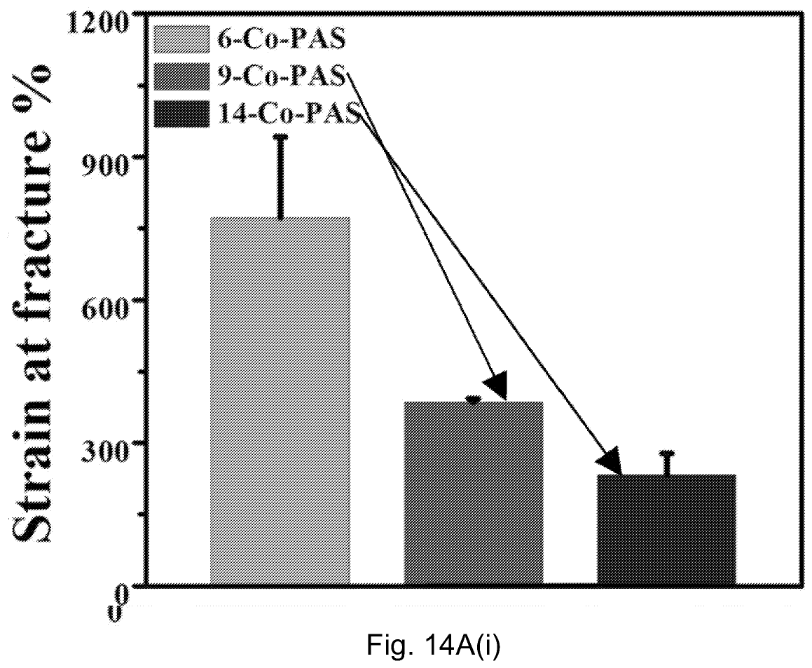

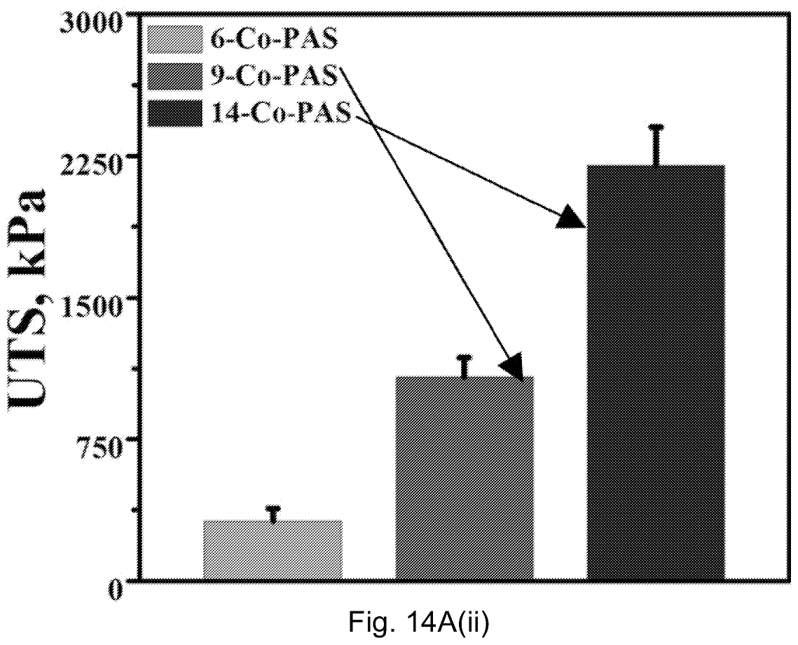

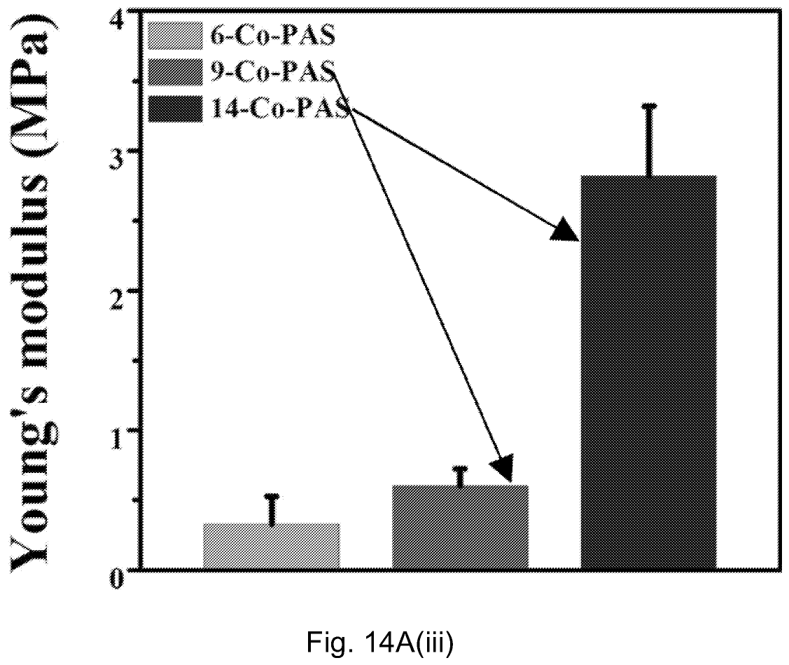

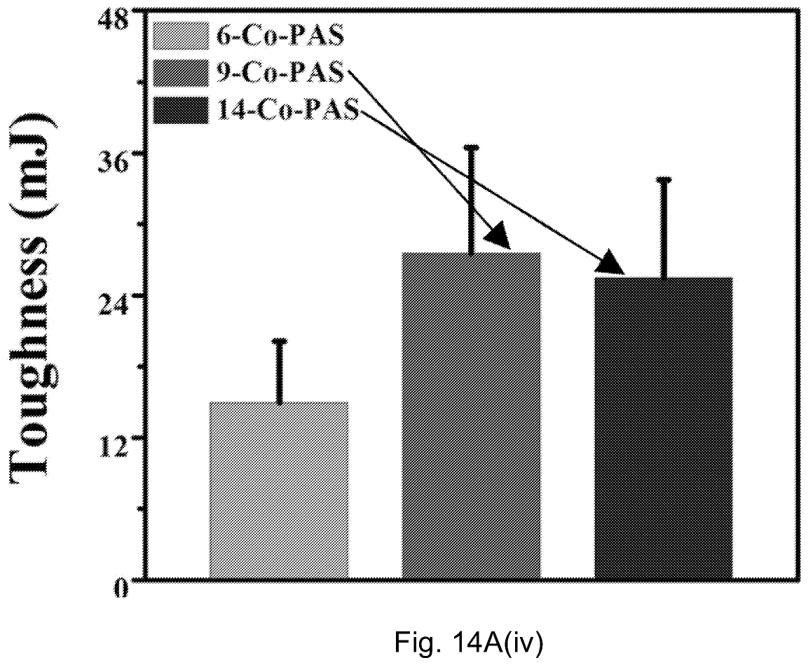

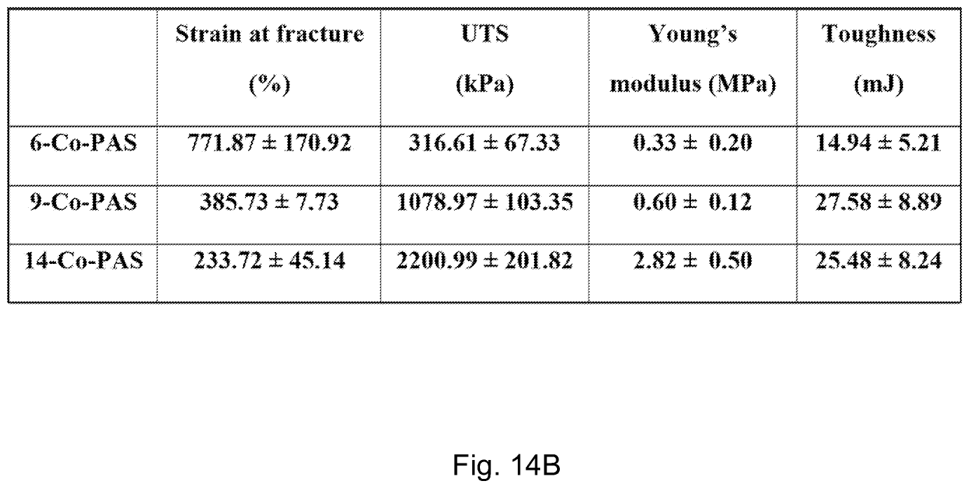

Ligand/metal ratio impacted mechanical properties of M-PAS elastomers. To examine the feasibility of controlling mechanical properties by the number of crosslinks, 9-Fe-PAS elastomer was chosen as a representative of the M-PAS elastomers. The molar ratios of the ligand in 9-PAS elastomer to Fe³⁺ ranged from 1 to 6. In the UV-Vis spectra (FIG. 12A(ii)), the absorption at 327 nm of 9-PAS represented the π→π* transition, and the formation of 9-Fe-PAS elastomer shifted the π→π* transition from 327 nm to 328 nm (ligand/Fe³⁺=1), 326 nm (ligand/Fe³⁺=2) and 325 nm (ligand/Fe³⁺=3, 4, 5 and 6). The peak shifts of ligand/Fe³⁺ ratios of 1 and 2 were smaller than those of others. This implied that there were excessive salicylaldimine side groups that were not involved in the chelation when the molar ratio of ligand to Fe³⁺ is 1 or 2. The absorption remained the same beyond ligand/Fe³⁺ of 3 because Fe³⁺ is hexacoordinate and no more chelation bond formed with increasing amounts of ligands. The bands at 350-400 nm of 9-Fe-PAS elastomer with different ligand/Fe³⁺ ratios were due to charge-transfer in the complex, and the weak board bands at 450-650 nm were due to d→d transition of the Fe³⁺ ion, both were absent in 9-PAS. Evaporation of solvent and heating at 150° C. for 8 hours produced 9-Fe-PAS elastomer films. Tensile stress curves of 9-Fe-PAS elastomer showed that decreasing the Fe³⁺ amount resulted in softer and more stretchable films (FIGS. 12A(iii), 14A(i)-14(iv), 14B). When the molar ratio of ligand/metal increased from 2 to 6, the strain at break increased from 144±9.20% to 394±42.6%, the ultimate tensile strength (UTS) decreased from 1.13±0.06 MPa to 0.55±0.06 MPa, the Young's modulus (E) decreased from 2.13±0.19 MPa to 0.67±0.12 MPa and the toughness remained at 12 mJ (FIG. 14B). This is because fewer crosslinkers led to longer polymer chains between the crosslinks, resulting in a more stretchy elastomer.

Ligand density controls mechanical property of M-PAS elastomer. Co-PAS elastomer films with varied ligand density were prepared and used for tensile tests to examine the mechanical property. To simplify, the ligand/metal ratio was fixed at 2. Tensile tests on the three variants of Co-PAS elastomer revealed stress-strain curve characteristics of elastomeric materials with a wide range of mechanical properties: a 3.30-fold range of strain, 6.95-fold of stress and 8.55-fold of modulus (FIGS. 12B, 14A(i)-14A(iv), 14B). With an increase of ligand density from 6% to 9% and 14%, the UTS and Young's modulus increased and the strain at fracture decreased, consistent with an increase of crosslinking density: the UTS increased from 316±67 to 2200±201 kPa; Young's modulus increased from 0.33±0.20 to 2.82±0.50 MPa; and the strain at fracture decreased from 772±171% to 45.1±7.73%. (FIG. 14B). On the other hand, the toughness peaked at 9% ligand density at 27.58±8.89 mJ. The same trend held for the other metals investigated. Therefore, with a certain metal ion, altering the ligand density of PAS controlled the mechanical properties.

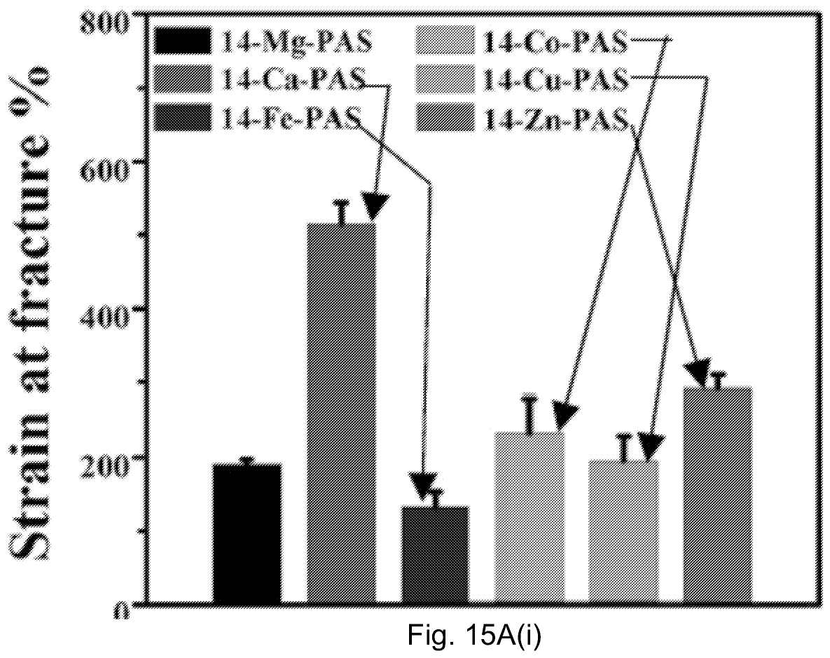

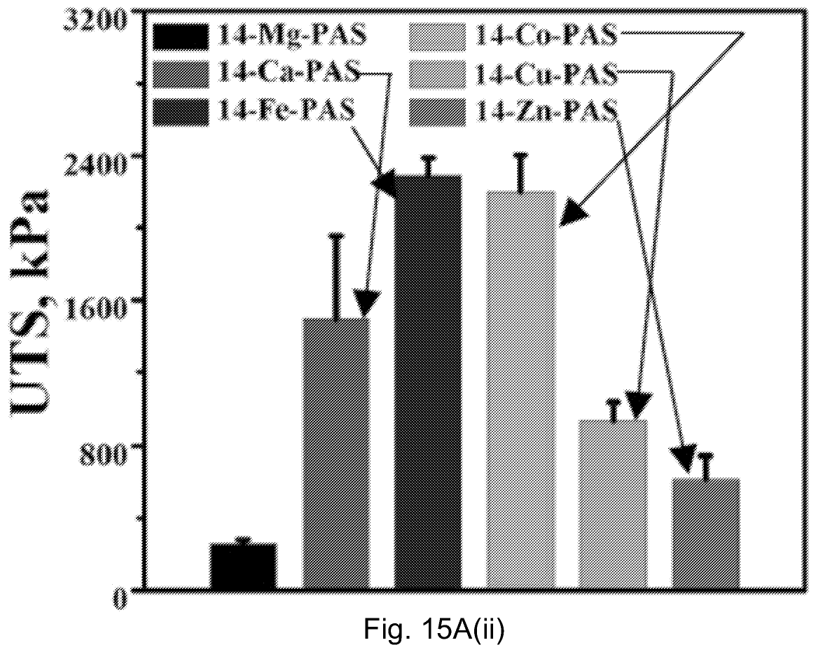

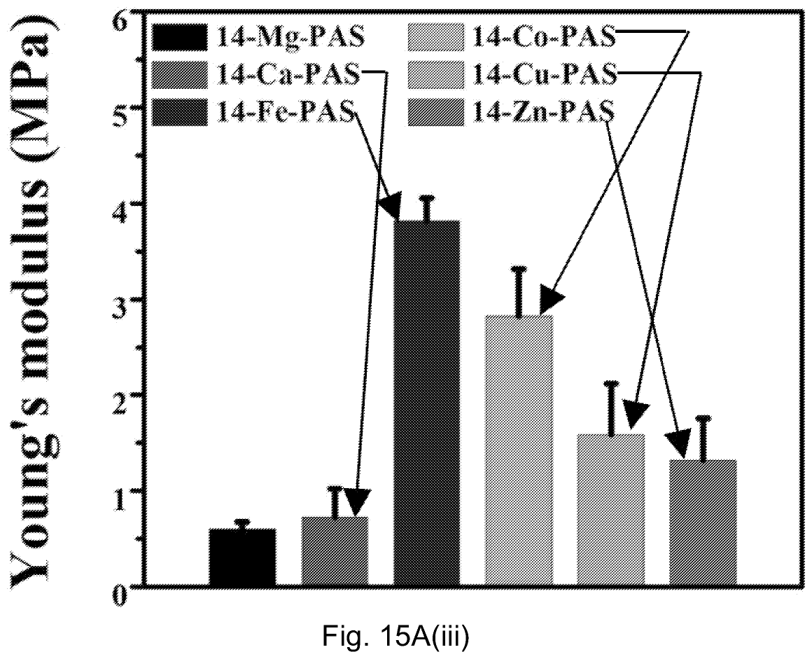

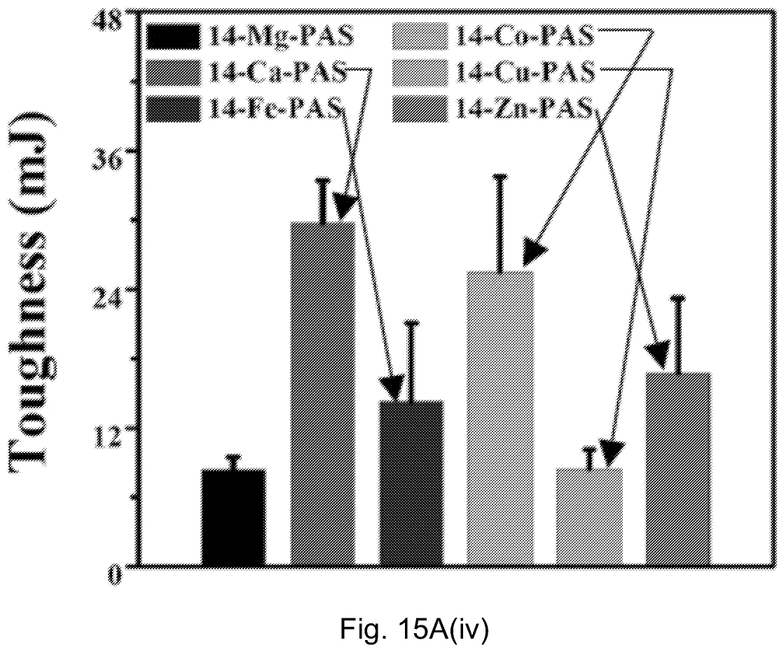

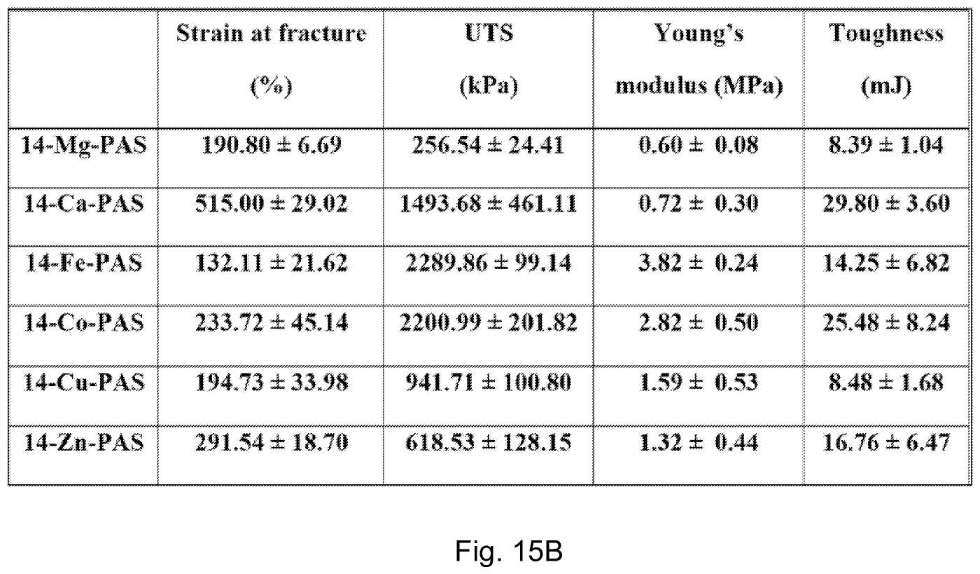

Metal ion types determine mechanical properties of M-PAS elastomer. With a given ligand density, different metals had different chelation bond strengths, offering an additional means to control the mechanical properties of M-PAS elastomer, including stretchability, stiffness, toughness and viscoelastic dissipation of the polymers. Using 14-M-PAS elastomer as an example, among the 6 metal ions tested, Ca²⁺ formed the toughest elastomer with a strain of break at 515.00±29.02%, UTS of 1493.68±461.11 kPa, modulus of 0.72±0.30 MPa and toughness of 29.80±3.60 mJ. 14-Fe-PAS elastomer had the smallest strain at 132.11±21.62%, highest UTS at 2289.86±99.14 kPa and highest Young's modulus at 3.82±0.24 MPa (FIGS. 12C, 15A(i)-15A(iv), 15B). The comparison among these metal ions is crucial for future selection of metal ions to obtain elastomers with specific mechanical properties to meet the demand of a specific application.

The elasticity of M-PAS. The ability to recover from mechanical deformation is key to the function of an elastomer in a mechanically dynamic environment and reduce potential mechanical irritation to the host during the tissue regeneration process. To this end, the hysteresis test was performed on 14-M-PAS elastomer films prepared with a ligand/metal ratio of 2. The strain to 20% (FIGS. 12D(i)-12D(vi)) was equal to or greater than what many soft tissues such as ligaments and arteries typically experience. All films underwent cyclic loading for at least 100 cycles without rupture. The hysteresis test revealed that metal ions determined the elastic recoil of 14-M-PAS elastomer. Mg²⁺ crosslinked polymer showed pronounced hysteresis loops with reduced stress as the cycles increased, which indicated energy dissipation from bond breakage. In contrast, polymers crosslinked by other metal ions showed small hysteresis loops, indicating little damage occurred during cyclic loading. Fe crosslinked polymer was the most elastic among those tested, reflecting strong chelation of Fe³⁺ to PAS. The elasticity was attributed to the rapid dynamic association and dissociation of the chelation bonds between metal ions and the salicylaldimine side groups of PAS under deformation. This dissipated the loading stress efficiently enabling the high capacity of M-PAS elastomer to tolerate deformations.

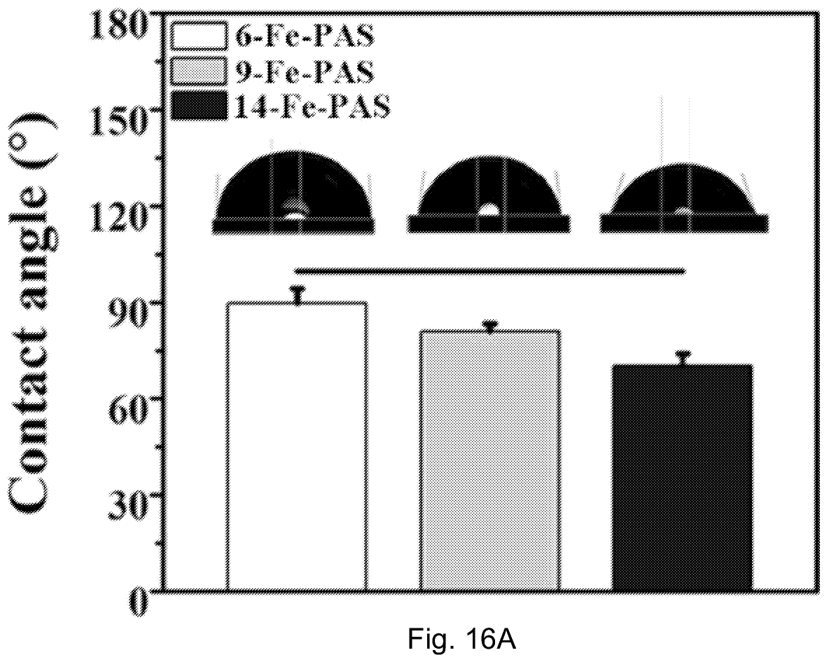

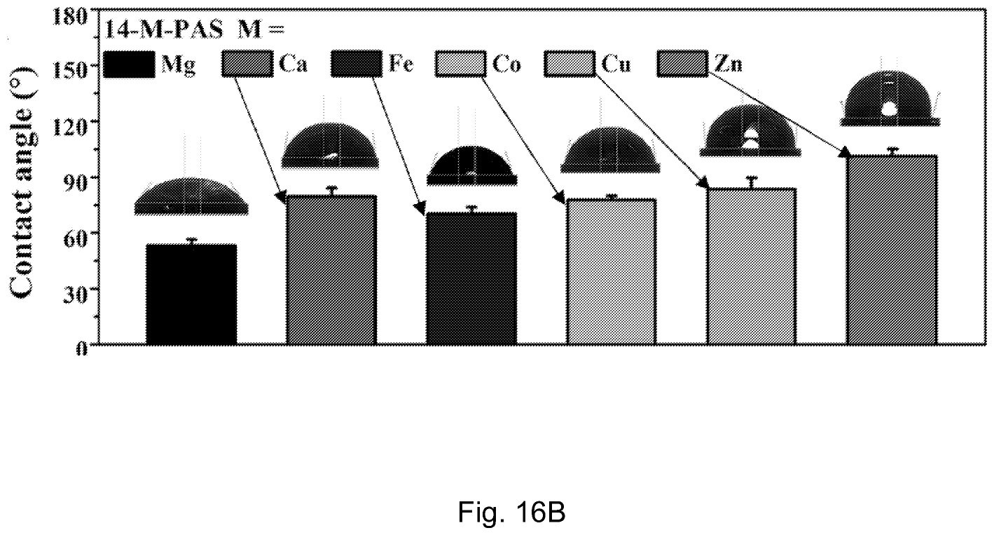

The hydrophilicity of M-PAS elastomer. Hydrophilicity is an important attribute of biomaterials. The hydrophilicity of M-PAS elastomer films was investigated by measuring the water-in-air contact angle (FIGS. 16A-16B). Different ligand amounts of PAS and types of metal ions led to different hydrophilicity. Contact angles of a 6-Fe-PAS, a 9-Fe-PAS and a 14-Fe-PAS elastomer were 89.70±4.61°, 80.93±4.61° and 70.33±3.67°, respectively, indicating the hydrophilicity of the films increased with increasing amounts of the ligand. This was likely because of the hydrophilicity of the salicylaldimine side groups. Overall, 14-M-PAS elastomer films were hydrophilic with the contact angles ranging from 53° to 83° (but it's 101.17±4.09° for 14-Zn-PAS elastomer), higher than a previously reported biomedical elastomer, PGS, thus the hydrolysis rate will likely be slower than PGS. As a comparison, the most widely used silicon-based organic polymer, polydimethylsiloxane (PDMS), is hydrophobic with a contact angle of around 100˜120°. Treatments such as plasma are needed to render the PDMS surface hydrophilic. Polyurethane, another important class of elastomer, has a contact angle ranging from 75˜95°. The hydrophilicity of 14-M-PAS elastomer films was close to that of poly(ether-urethane-urea)s, with a contact angle range of 67-87°. In summary, x-M-PAS elastomer was generally hydrophilic. The ligand density and metal ions types controlled the hydrophilicity, allowing adjustment of wettability and hydrolytic degradation rate of an x-M-PAS elastomer to suit a specific application.

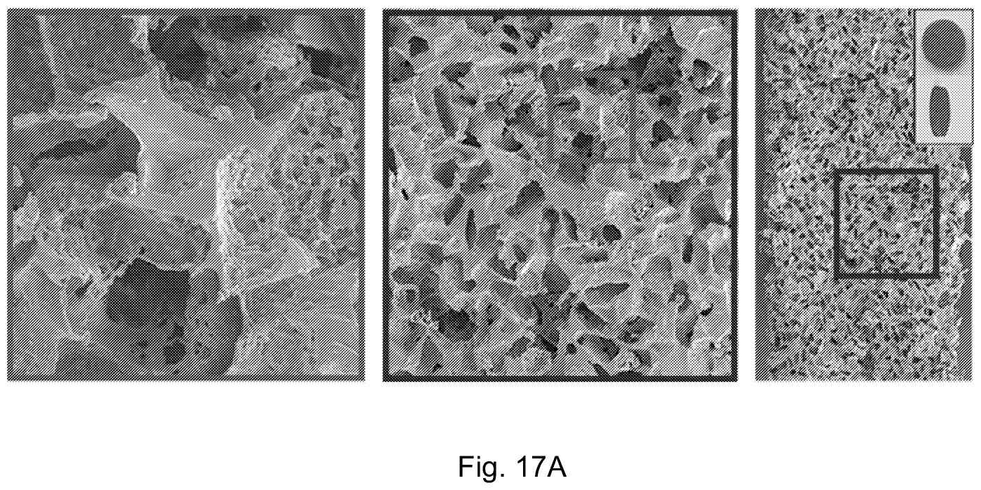

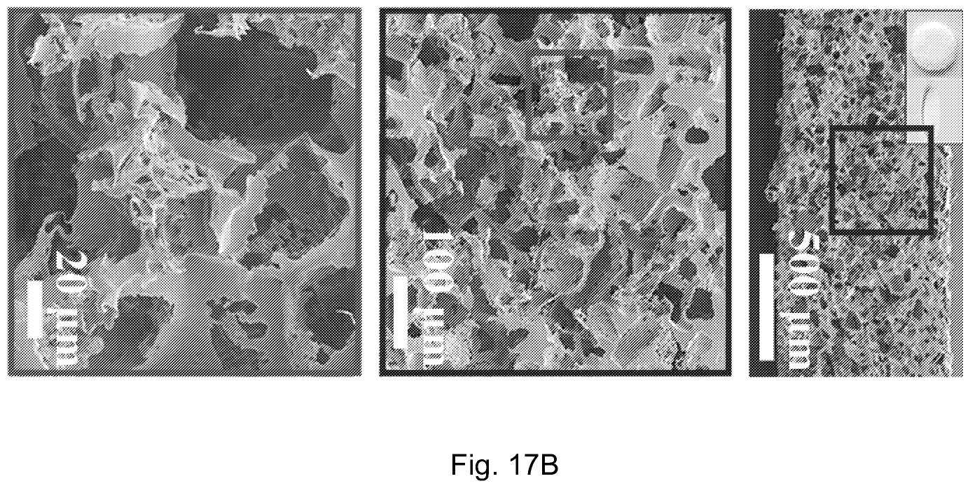

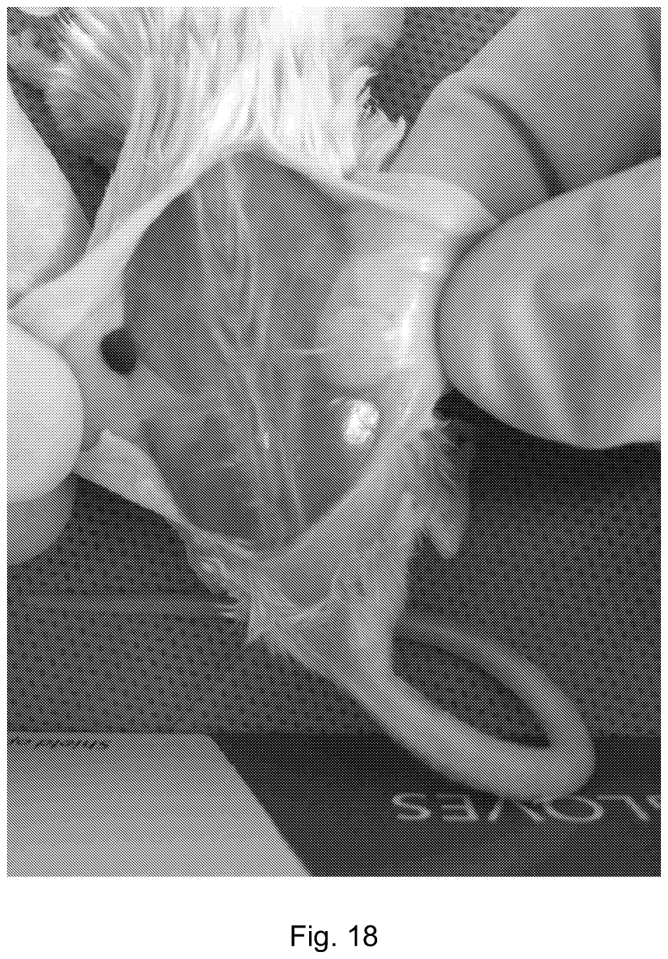

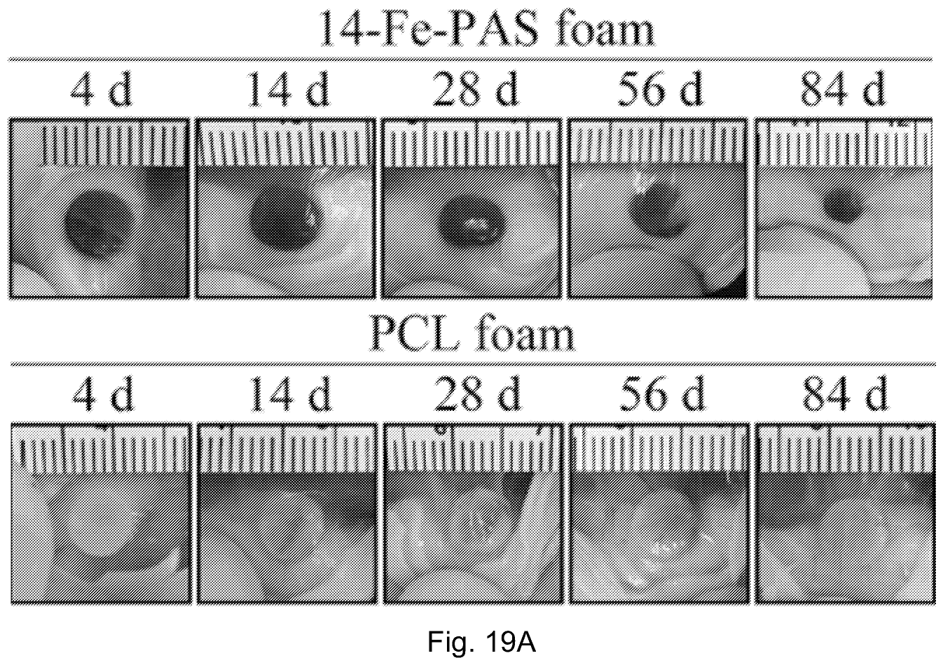

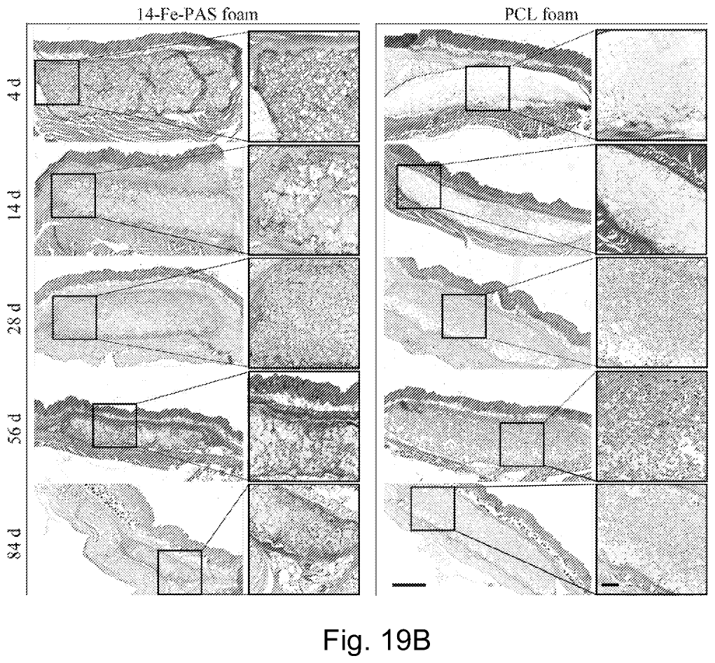

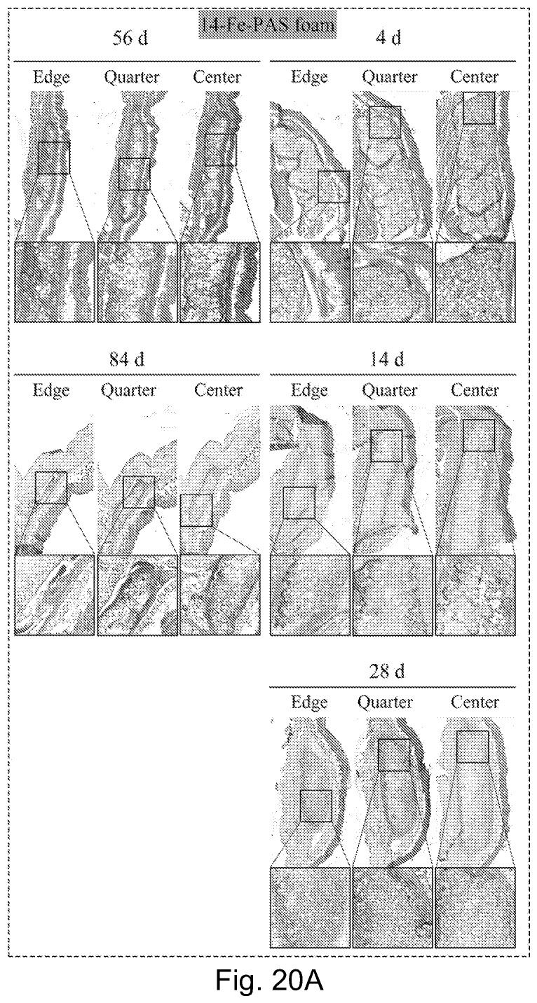

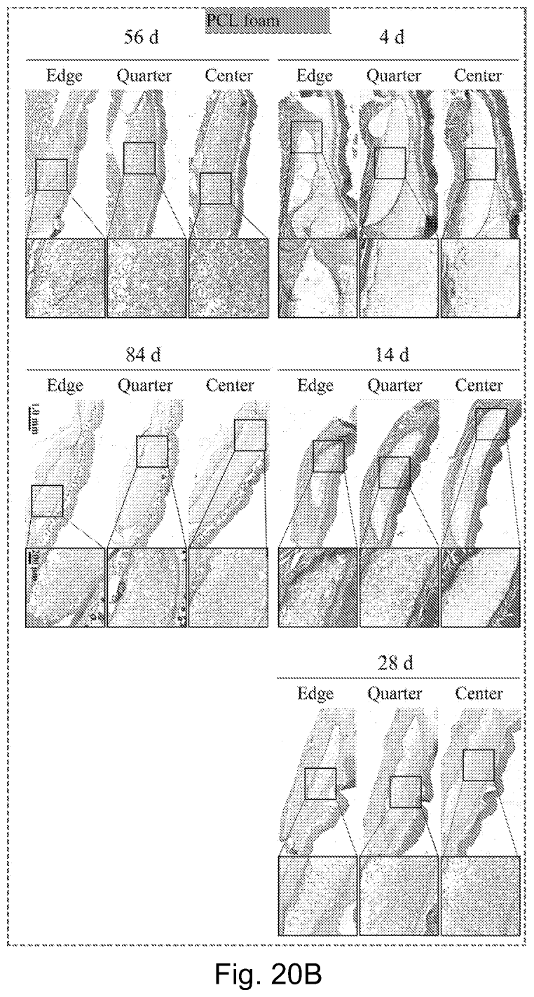

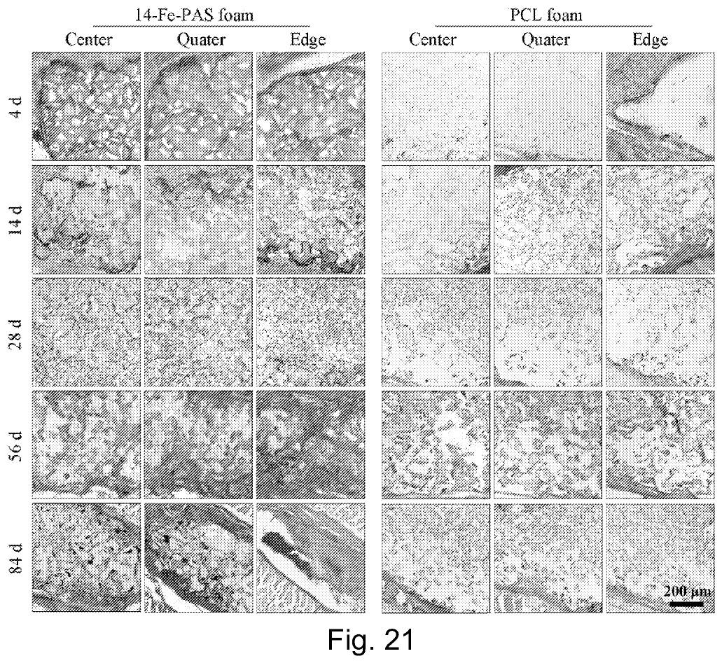

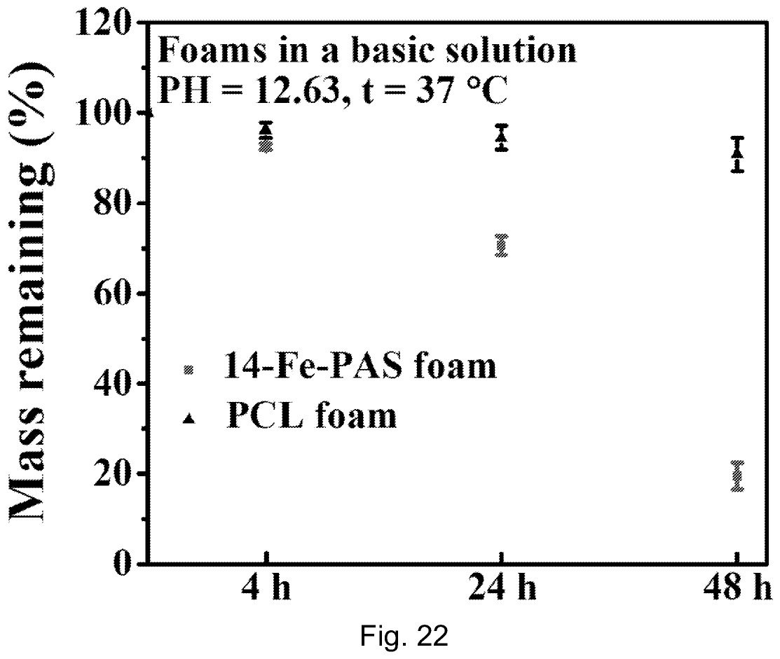

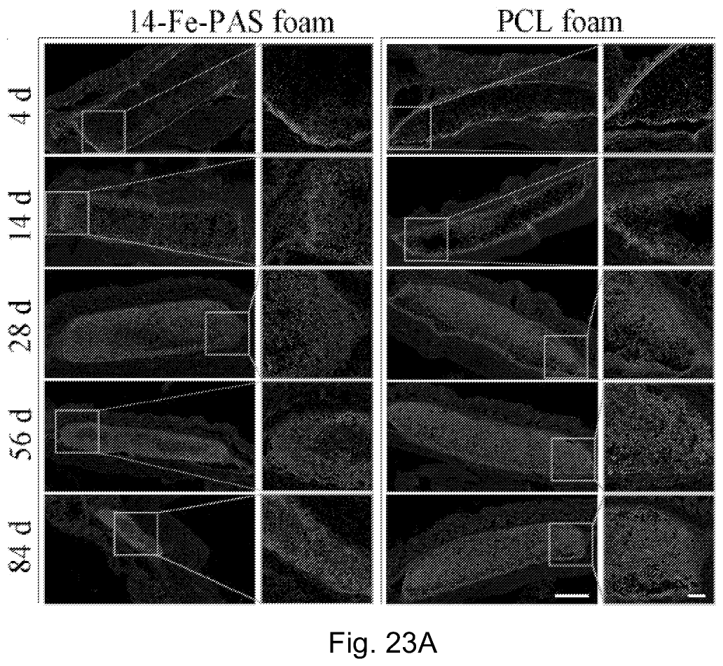

Biodegradability and Biocompatibility in vivo. Using 14-Fe-PAS elastomer as an example, its biocompatibility was evaluated via a subcutaneous (s.c.) implantation model in mice with poly(ε-caprolactone) (PCL, Mn=80,000 Da) as a control. The elastomer foams were prepared by a salt leaching method and had a similar pore structure (FIGS. 17A-17B) with a porosity of ˜62%. One 14-Fe-PAS elastomer foam and one PCL foam of identical size were implanted symmetrically into the back of the same mouse (FIG. 18). All mice survived without malignancy, infections or abscesses at the implantation sites (FIGS. 19A-19B, 20A-20B, 21). For 14-Fe-PAS elastomer foams, the H&E staining revealed cells only at their surfaces with no sign of degradation 4 days after implantation. After 14 days, cells infiltrated deeper and the implants showed visible rounding at the edge, likely because of degradation. On day 28, cells infiltrated the entire implants with fibrovascular tissues and the degradation of the bulk started, as indicated by the reduced presence of polymers in histological images. However, the implants maintained their original shape, indicative of mechanical integrity. Histological analysis suggested that most of the elastomer had degraded by day 84. The control PCL foams also showed cells infiltration after 14 days of implantation. The bulk of the PCL foams started a limited degradation afterward. PCL implants retained their shape with little dimensional change after 84 days. 14-Fe-PAS elastomer degraded faster than PCL in vivo and exhibited a 4.67 times higher degradation rate than PCL in vitro (FIG. 22).

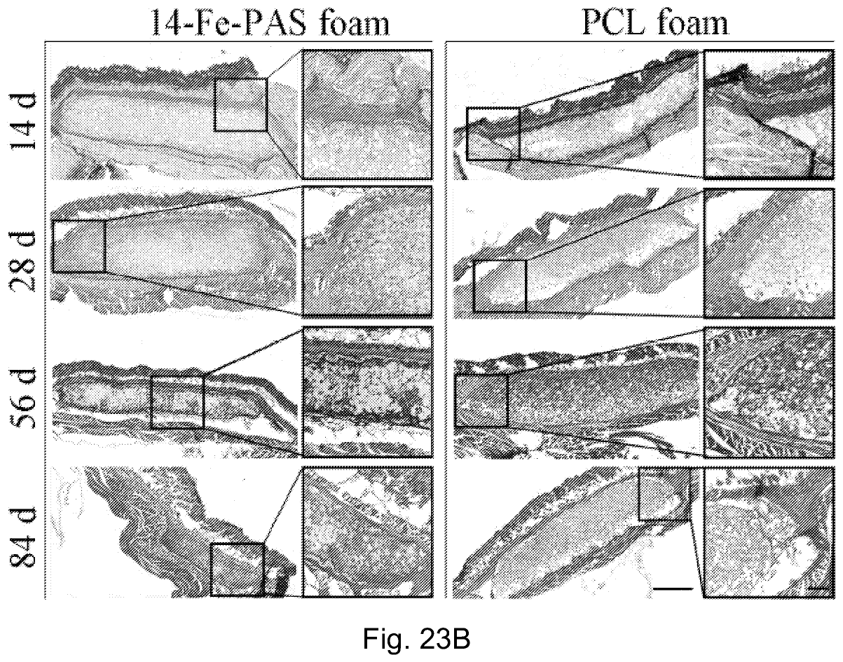

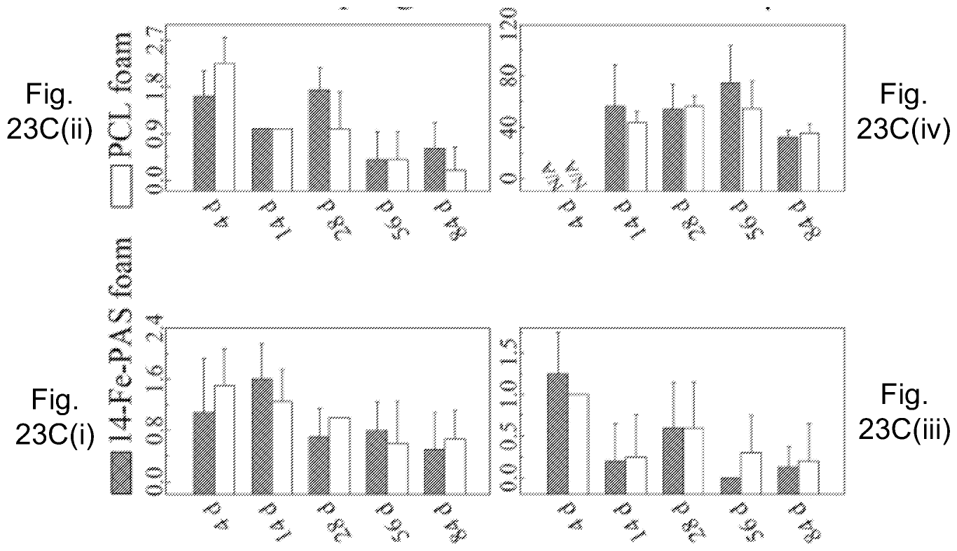

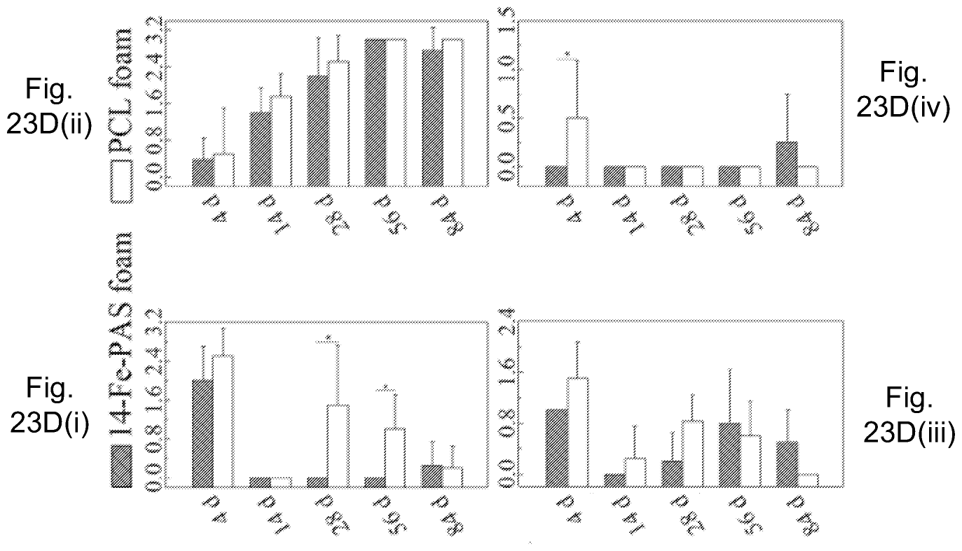

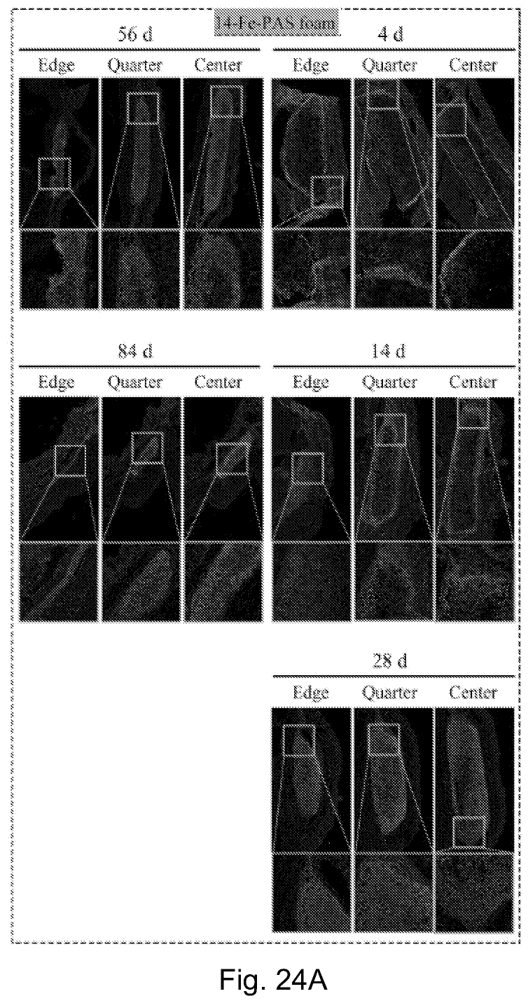

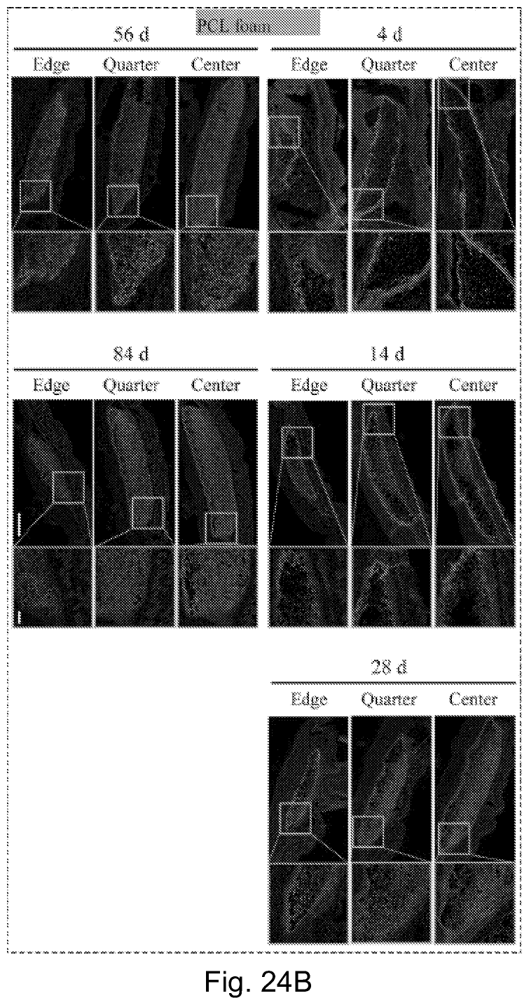

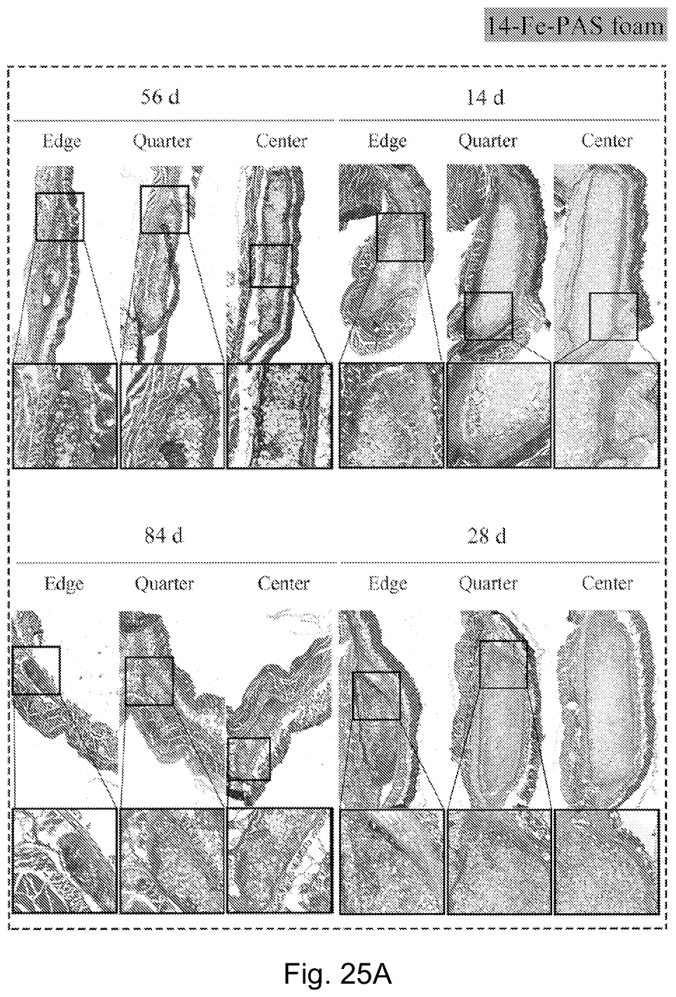

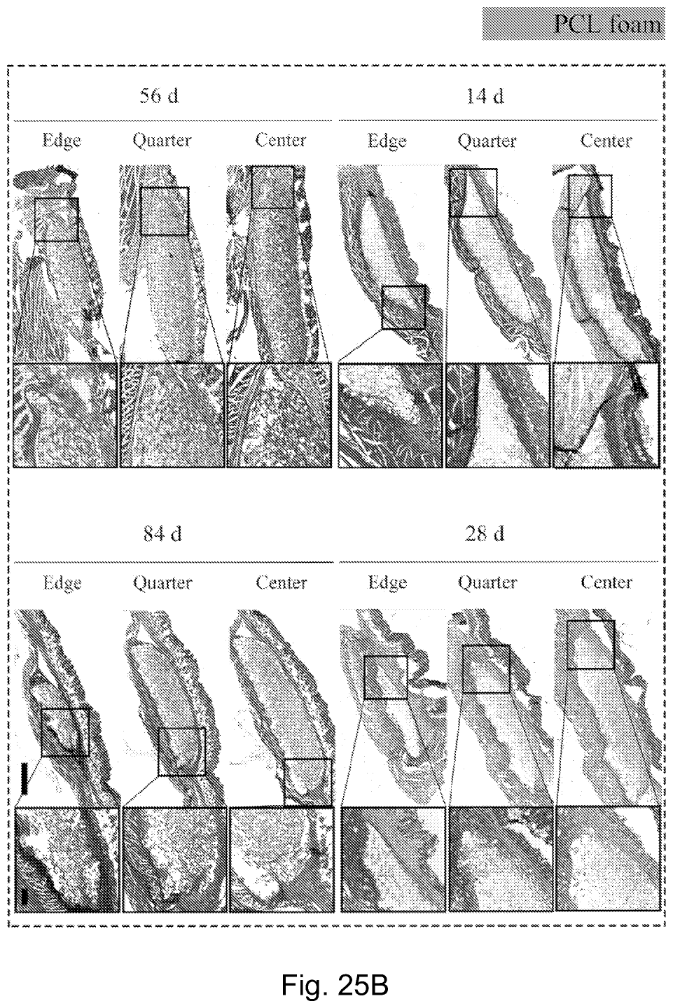

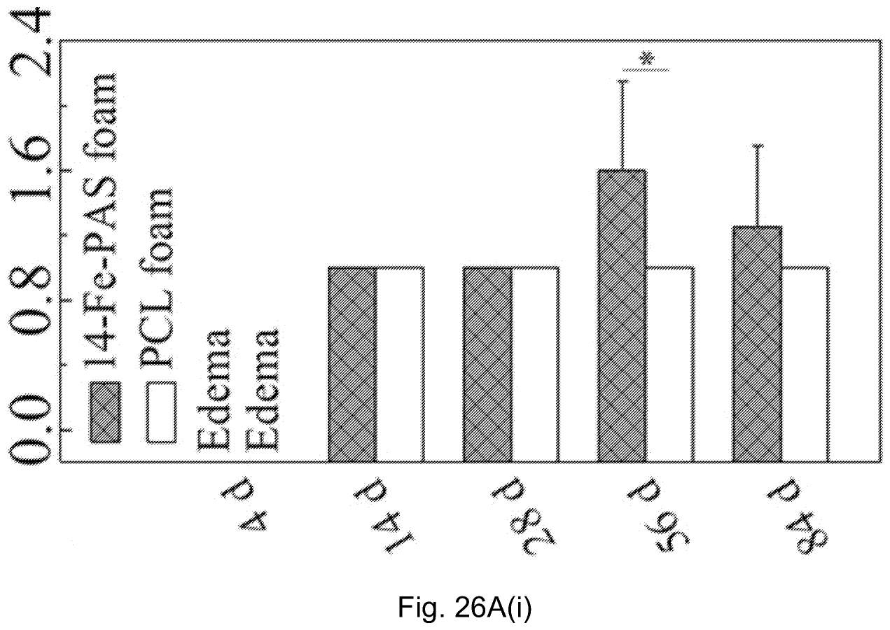

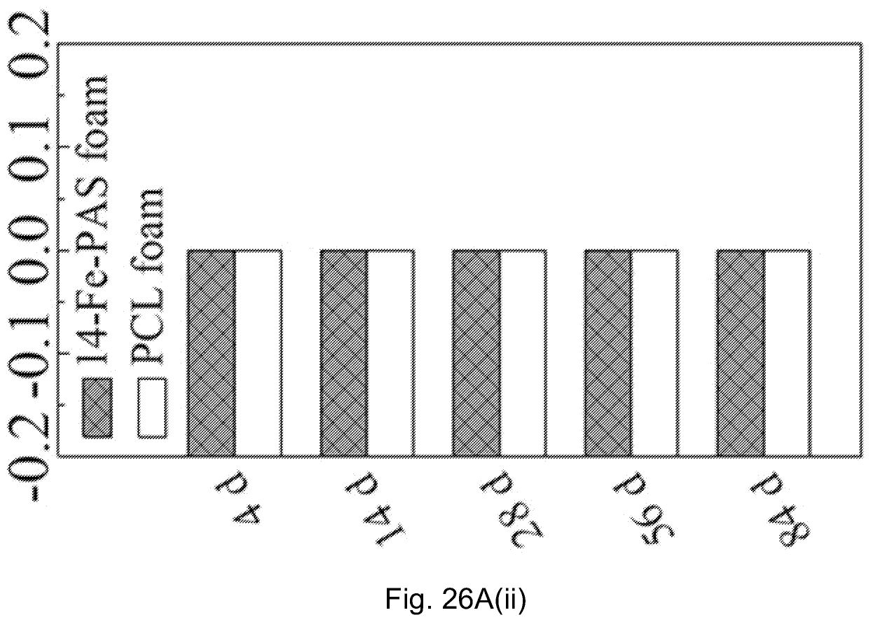

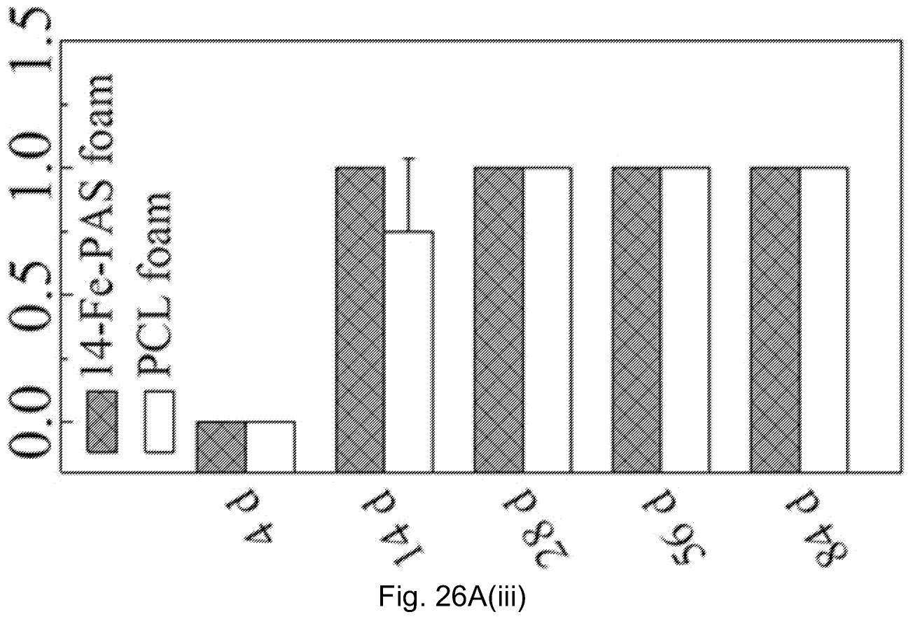

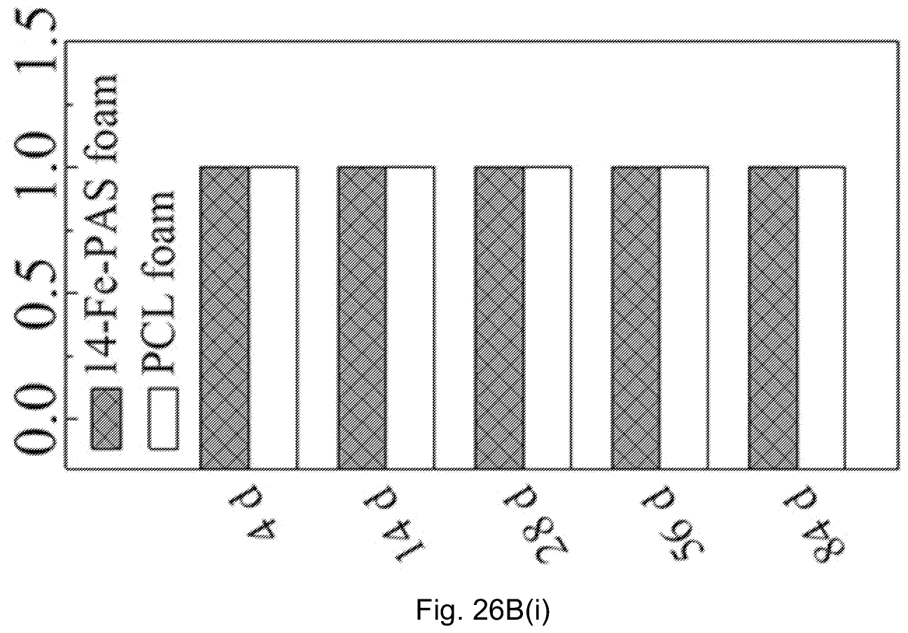

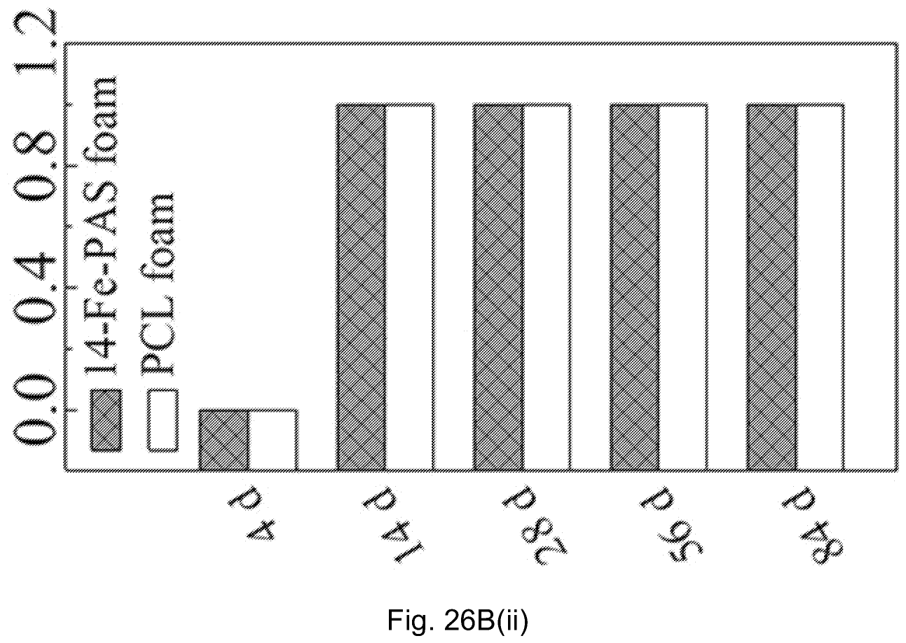

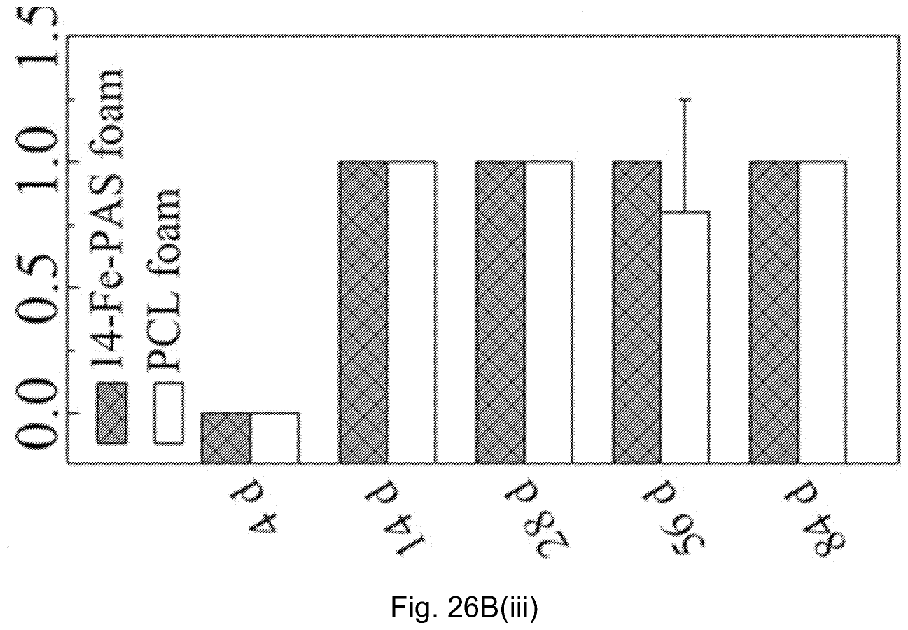

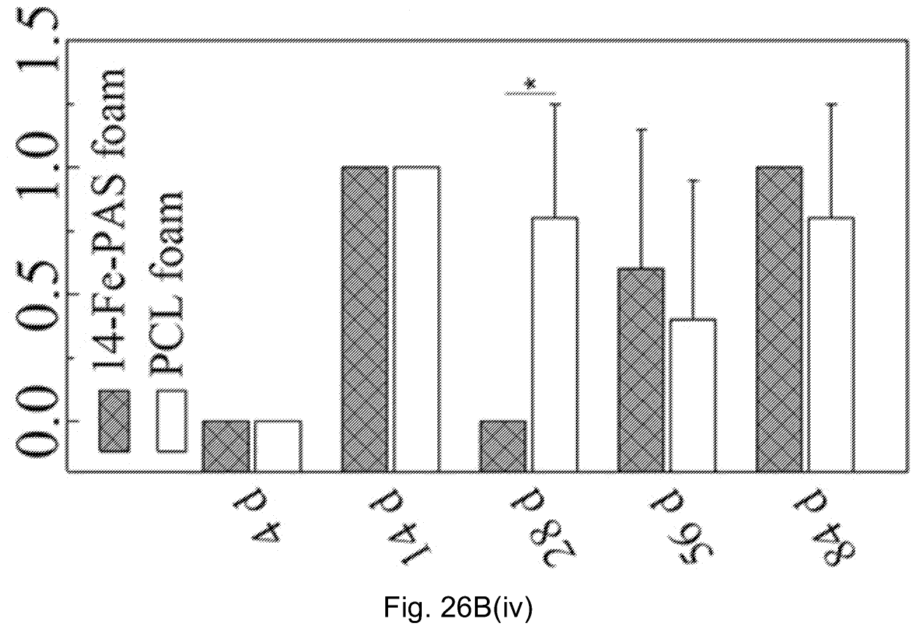

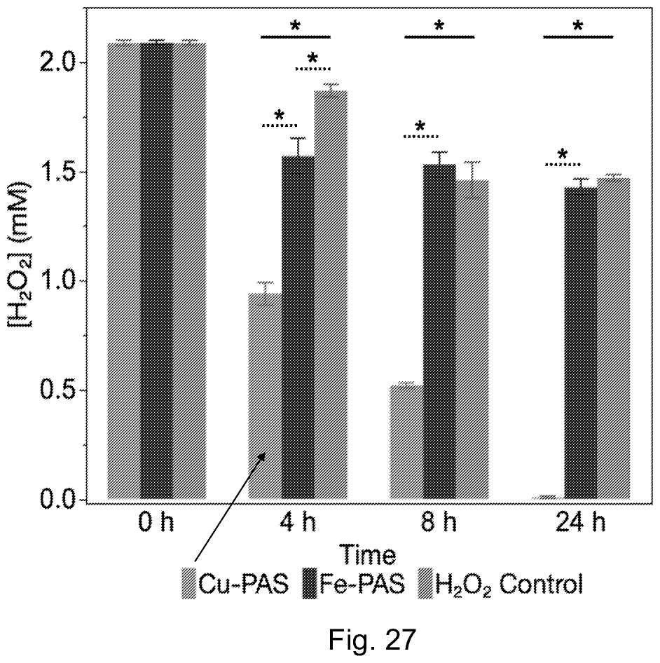

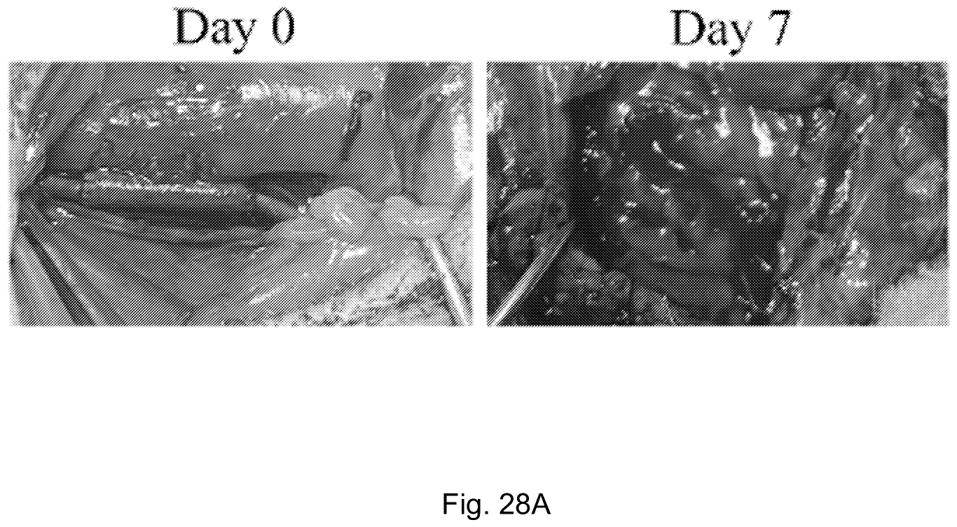

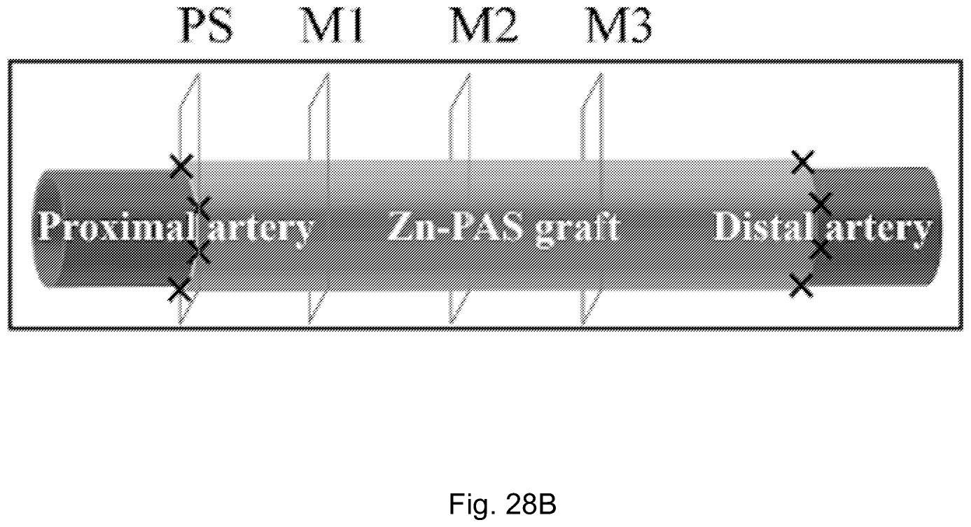

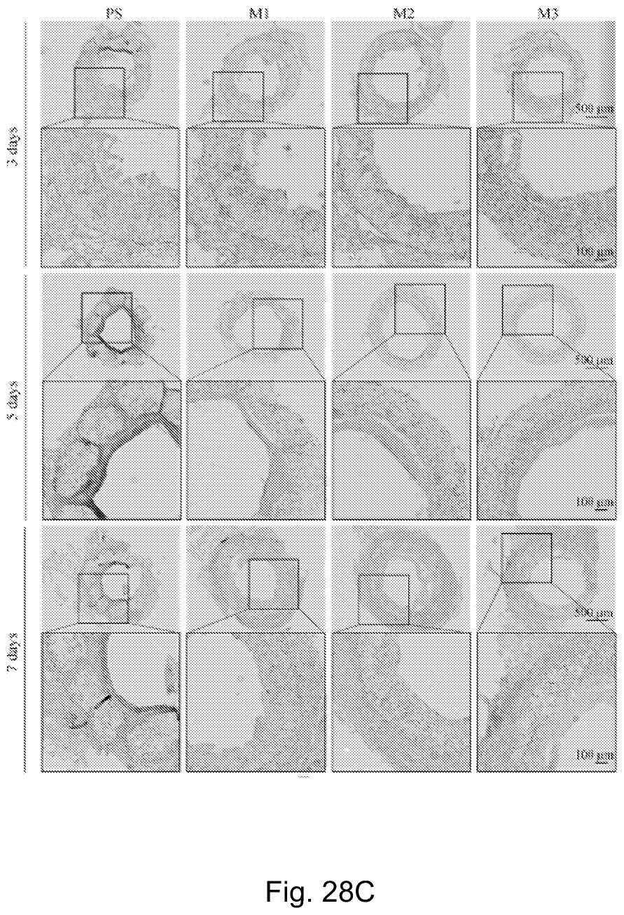

Tissues around both 14-Fe-PAS elastomer and PCL implants showed mild adverse responses such as inflammation and fibrosis (FIGS. 19A-19B, 23A-23B, 23C(i)-23C(iv), 23D(i)-23D(iv), 20A-20B, 21, 24, 25A-25B, 26A(i)-26A(iii), 26B(i)-26B(iv)). Inflammatory cells recruited in the surface areas of all implants at day 4 because of a nonspecific inflammatory response to the implants, and then migrated into and proliferated inside the implants. Throughout the observation window, the number of granulocytes in tissues surrounding 14-Fe-PAS elastomer and PCL implants showed no difference, while there were a significantly higher number of granulocytes inside the PCL implants than those inside 14-Fe-PAS elastomer at 28 and 56 days (FIGS. 19B, 20A-20B, 21, 23C(i), 23D(i)). Macrophages, key mediators of inflammation and wound healing, showed a slightly higher presence in the surrounding tissues of PCL than in 14-Fe-PAS elastomer at day 4 (FIGS. 23B, 23C(ii), 23D(ii)). There were larger numbers of macrophages inside of the PCL implants than 14-Fe-PAS elastomer at all time points. Fibroblasts play an important role in wound healing after implantation. The number of fibroblasts in tissues surrounding 14-Fe-PAS elastomer at day 56 was significantly higher than that of PCL, while the infiltrating fibroblasts inside the implants were at a similar level in both groups (FIGS. 26A(i), 26B(i)). The lymphoplasmacytic (LC-PC) inflammation, as a type of chronic inflammation consisting of lymphocytes and plasma cells (terminally differentiated B lymphocytes), was also scored but no difference showed in 14-Fe-PAS elastomer and PCL. Overall, the PCL group showed slightly stronger inflammation than the 14-Fe-PAS elastomer group (the former even showed muscle degeneration or necrosis in half of the implants at day 4, see FIG. 23D(iv)). 14-Fe-PAS elastomer degraded faster than PCL, presenting a higher concentration of degradation products in the microenvironment of the implant site (note: our intention was to match the degradation rate). Thus, the inflammatory responses to PCL would likely be stronger if the degradation of PCL matches that of 14-Fe-PAS elastomer. Collagen deposition is part of the wound healing response to an implant. A fibrous capsule surrounded the 14-Fe-PAS elastomer and PCL foam from day 14 onward (FIGS. 23B, 25A-25B). The capsule thickness was similar for both materials at all time points. Moreover, the fibrillar connective tissues in the surrounding tissue (FIG. 26A(iii)) and collagen within the implants (FIG. 26B(iii)) showed no difference in 14-Fe-PAS elastomer and PCL. Overall, the inflammatory response to 14-Fe-PAS elastomer was milder than to PCL in the subcutaneous environment.

This example demonstrated the principle of using metal chelation bonds to crosslink elastomers with a wide range of mechanical properties controlled by the type of metal ions, metal to ligand ratios, and ligand density in the polymers. The biocompatibility of the elastomers matched that of PCL, opening promising new avenues for elastomer development for improved soft tissue reconstruction and regeneration. In particular, the high degree of elastic recoil would be advantageous in tissues experiencing large deformations, such as ligaments, blood vessels, skin, lung, kidney, and heart.

Materials and methods. Synthesis of HPA monomer. The Schiff-base ligand HPA (2-[[(2-Hydroxyphenyl)methylene]amino]-1,3-propanediol, FIG. 1A(i)) was synthesized by a simple one-pot reaction. Briefly, 20 mmol of 2-amino-1,3-propanediol (Serinol, Sigma-Aldrich) was added to a methanolic solution (200 mL) of salicylaldehyde (20 mmol, Sigma-Aldrich). 20 mmol of triethylamine was added dropwise to the above mixture and then the mixture was stirred for 5 hours at room temperature to obtain a yellow solution. The solvents of the solution were removed using a Buchi Rotavapor (R-300, Cole-Parmer®). The viscous product was then dissolved in 15 mL of ethyl acetate and kept at −20° C. overnight for crystallization. The crystals of the product were collected by vacuum filtration and then dried at 30 mTorr and 30° C. for 24 hours to obtain a yield of 91.2%.

Synthesis of PAS polymers. PAS polymers with different densities of ligands (salicylaldimine side groups) were synthesized by polycondensation of 1, 3-propanediol (VWR International), HPA and sebacic acid (Sigma-Aldrich) and adjusting the molar percentage of HPA in the diols (FIG. 1A(ii)). For 6-PAS (HPA: 1,3-propanediol: sebacic acid=15 mol %: 85 mol %: 100 mol %), 17 mmol of 1,3-propanediol was first reacted with 20 mmol sebacic acid at 120° C. under argon for 24 hours. The reaction mixture was kept at 10 mTorr and 120° C. for 24 hours with magnetic stirring and then 3 mmol of HPA was added. The mixture was further reacted at 120° C. under argon for 16 hours and then kept with magnetic stirring at 10 mTorr and 120° C. for 12 hours. The reaction solution was decanted into a centrifuge tube, cooled down to room temperature and then placed at 4° C. for further use. The yield of 6-PAS was 94.6 wt %. The 9-PAS and 14-PAS were similarly synthesized using 20 mol % or 25 mol % of HPA.

Characterization of HPA monomer and PAS polymer. The HPA was dissolved in methanol in a concentration of 100 ppm and the molecular weight was identified by gas chromatography-mass spectrometry (GC-MS, JEOL GCMate). The UV-visible spectra of HPA and Cu(HPA)₂ solutions were collected using the SpectraMax M3 microplate reader (Molecular Devices, Sunnyvale, CA). The acetone and ethanol were used as the solvents for HPA and copper (II) chloride anhydrous (VWR International), respectively. The ¹H-NMR (500 Hz, Bruker AV500) spectroscopic technique was used to examine the chemical structure of HPA, 6-PAS, 9-PAS and 14-PAS, as well as the metal chelation between HPA and copper acetate. The acetone-d₆ was used as the solvent. A Q1000 modulated differential scanning calorimeter (MDSC) was used for DSC measurement of the 6-PAS, 9-PAS and 14-PAS polymers. The molecular weight of the intermediate reaction products of 1,3-propanediol and sebacic acid, of which the 1,3-propanediol: sebacic acid=0.85:1, 0.80:1 or 0.75:1, as well as the 6-PAS, 9-PAS and 14-PAS was determined by gel permeation chromatography using Malvern Panalytical OMNISEC GPC system (Malvern Instruments Ltd, UK), equipped with triple detectors, including refractive index, right angle and low angle light scattering (RI, RALS and LALS). The column set of T6000M and T3000 and THF were used as stationary and mobile phases, respectively. The polymers were dissolved in HPLC grade THF at 5.0 mg/ml and filtered through a 0.2 μm PTFE syringe filter for the test. The detection was replicated thrice.

Preparation of M-PAS elastomer films, foams and porous tubes. Copper chloride anhydrous (CuCl₂, VWR International) in ethanol, calcium chloride (CaCl₂, Sigma-Aldrich) in H₂O/ethanol (1/5, v/v), zinc acetate dihydrate (Zn(AcO)₂·2H₂O, Macron Fine Chemicals) in methanol, iron chloride hexahydrate (FeCl₃·6H₂O, Sigma-Aldrich) in ethanol, magnesium acetate tetrahydrate (Mg(AcO)₂·4H₂O, Beantown Chemical) in ethanol, or cobalt acetate tetrahydrate (Co(AcO)₂·4H₂O, Alfa Aesar) in H₂O/ethanol (1/3, v/v) was added to 900 mg of the linear 6-, 9- or 14-PAS polymer dissolved in acetone (100 mg/mL) to get the M-PAS solution, respectively. The molar ratio of the ligand of PAS to metal ions was 2. The ligand density of PAS polymer was calculated based on the ¹H-NMR spectra. The M-PAS elastomer films were prepared by casting the mixtures of PAS and metal ions to silicone molds (width×length×thickness=22×32×1 mm) that were placed on PFA film-wrapped copper plates and, followed by vacuum drying at 60° C. overnight. The films were cured at 30 mTorr and 150° C. for 8 hours and then cooled to room temperature overnight before the test. The 9-Fe-PAS elastomer films with different ligand/metal molar ratios were prepared by changing the molar ratio of ligand:Fe³⁺ metal from 2, 3, 4, 5 to 6. Preparation of 9-Fe-PAS elastomer film with ligand/Fe³⁺ of 1 failed because gelation happened immediately after adding Fe³⁺ to 9-PAS solution and it was impossible to get a homogenous film.

The 9-Cu-PAS and 14-Fe-PAS elastomer foams were prepared by using a previously published salt-template leaching method, with NaCl particulates (32˜53 μm) as porogen. NaCl particulate was evenly spread into silicon molds and kept in 37° C. Hybridization Incubator (Robbins Scientific, Model 1000) for 90 minutes for salt fusion. The salt templates were then dried at 80° C. overnight and then the 9-PAS/Cu²⁺ or 14-PAS/Fe³⁺ mixture solution was added dropwise onto the salt templates. The samples were dried in air for 12 hours, followed by vacuum drying at 60° C. overnight. The samples were cured at 30 mTorr and 150° C. for 8 hours and then cooled to room temperature to yield 9-Cu-PAS or 14-Fe-PAS composites (polymer:NaCl=1:3, w/w). The NaCl particulate was removed by immersing samples in deionized water for 48 hours with the replacement of the water every 6 hours. The porous scaffolds were then freeze-dried. The 9-Cu-PAS elastomer porous tube was prepared similarly, with the NaCl particulate sizing 25˜32 μm as porogen, a stainless steel rod (0.8 mm in outer diameter) as a mandrel and a Teflon® tube (1.58 mm in inner diameter, 20 mm in length) as a mold.

Characterization of M-PAS elastomer films, foams and porous tubes.

The KBr pellets of newly prepared 9-PAS polymer and 9-Fe-PAS elastomer films were used for Fourier-transformed infrared (FTIR) spectrum analysis (Bruker Vertex V80V Vacuum FTIR system, PTIK Instruments) to confirm the metal chelation crosslinking between ligand of 9-PAS and Fe³⁺ metal. The UV-visible spectra of 9-PAS and 9-Fe-PAS elastomer solutions with molar ratios of ligand/Fe³⁺ ranging from 1 to 6 were collected using the SpectraMax M3 microplate reader. The hydrophilicity of the M-PAS elastomer films was evaluated by a contact angle measurement. The water-in-air contact angle was measured by the sessile drop method with a Rame-Hart 500 contact angle goniometer (Rame-Hart Inc., NJ) at room temperature. The pore structures of the 9-Cu-PAS elastomer porous tubes, 14-Fe-PAS foams and PCL foams were checked with a scanning electron microscope (Tescan Mira3 FESEM, Brno, Czech Republic). The cross-sections of the porous tubes or foams were sputter-coated with gold-palladium for 30 s in a Denton Vacuum Desk V (Denton Vacuum Inc.) before observation. The porosity of the 14-Fe-PAS elastomer and PCL foams scaffolds was calculated according to Archimedes' principle (n=4) by the following formula: porosity=Vₚₒᵣₑ/V=((W₂−W₁)/ρ/V)×100%, where Vₚₒᵣₑ is the volume of the pore, V is the total volume of the foams, W₁ is the dry weight of the foams, W₂ is the weight of foams with water and p is the density of water. The porosity and pore size distribution of the 9-Cu-PAS elastomer porous tube was measured using X-ray micro-computed tomography (micro-CT) as previously published. The elastomer tube was scanned using an Xradia Zeiss VersaXRM-520 micro-CT (Carl ZEISS AG, Germany) and the three-dimensional (3D) images were reconstructed using an Avizo lite 9.7.0 reconstruction software (Thermo Fisher Scientific, MA).

Mechanical tests. The Dog-bone samples were punched from the crosslinked M-PAS films using a dog-bone cutter with the neck dimensions of 1.6×10.5 mm. Tensile and hysteresis tests were performed at room temperature according to the standard method (ASTM D412) using a Universal Testing System (Instron 5943, Instron Engineering Corp., MA) equipped with a 50 N loading cell. The elongation rate was set at 20 mm/min for tensile tests and 30 mm/min for the cyclic stress-strain test. Values of the strain at fracture (%), ultimate tensile strength (UTS), Young's modulus (E) and toughness (mJ) were presented as the mean±standard deviation according to data of ≥4 samples. The Young's modulus was calculated from the low-strain region (<20% strain) of the stress-strain curve. The toughness was obtained by calculating the area under the Force-Tensile displacement curve, where the domain ends at Break (Cursor). The hysteresis tests were repeated four times to obtain an average cyclic loading number for each sample.

Degradation and metal extraction tests. An in vitro degradation test was performed under the basic condition for the 14-M-PAS films. Disk-shaped samples (Φ6 mm×H1 mm) were weighed and placed in the PBS solution with 60 mM NaOH (pH=12.63, 5 ml PBS per sample) and then incubated on a rotating shaker at 37° C. On designated time points (4, 24 and 48 hours), samples were retrieved, washed 5 times with deionized water, and then lyophilized for 24 hours. The mass of the remaining samples was measured using a microbalance. The degree of degradation was determined by dry weight change. Three replicates were performed and the values averaged.

For the Fe³⁺ extraction from 14-Fe-PAS elastomer gel, 100 mg 14-PAS was first dissolved in 0.5 mL acetone then the Fe³⁺ was added dropwise into the 14-PAS with ligand/Fe³⁺ ratio of 2. The PAS solution gelled immediately under agitation at room temperature. An EDTA Disodium salt (Promega™ Corp., WI) aqueous solution with a concentration of 100 mg/mL was then added into the 14-Fe-PAS elastomer gel in an EDTA/Fe³⁺ molar ratio of 1. For the metal ions extraction from 6-M-PAS elastomer films, the EDTA extraction solution was prepared by dissolving 10 mM EDTA Disodium salt in DMF/H₂O mixed solution (DMF:H₂O=1:1, v/v). The 6-M-PAS elastomer stripes (5 mm in length, 2 mm in width and 1 mm in thickness) were weighed and placed in the EDTA solution (1.5 ml per sample) and then incubated on a rotating shaker at 70° C. After 48 hours, samples were retrieved, washed, lyophilized and weighed. The metal extraction degree was evaluated by dry weight change. Three replicates were performed and the values averaged.

In vitro cytocompatibility study. The in vitro cytotoxicity assay was performed on the 14-Cu-PAS elastomer coating using Human umbilical vein endothelial cells (HUVECs, C2519A, Lonza) according to the manufacturer's instructions. The HUVECs (passage 5) were sub-cultured using endothelial cell growth medium that contained 2% FBS and VEGF (EGM-2 BulletKit, CC-3156 & CC-4147, Lonza). The cells were harvested using conventional trypsin/EDTA after reaching confluence and re-suspended in EGM-2 to prepare a cell suspension solution of 5×10⁵ cells per mL for cell seeding. 1% w/v of 14-Cu-PAS elastomer in acetone was prepared and 20 μL of the solution was evenly spread on each of the coverslips (12 mm diameter). 20 μL of 1% w/v acetone solution of poly(D, L-Lactide-co-Glycolide) (PLGA, 50:50, ester terminated, M_{w} 7,000-17,000, Sigma-Aldrich) was coated on each of the coverslips to prepare the controls. The 14-Cu-PAS elastomer and PLGA coatings with a thickness of approximately 200 nm were formed on the coverslips after the coverslips were air-dried for 24 hours and further cured in a vacuum oven at 30 mTorr and 150° C. for 8 hours. The coated coverslips were placed into 24-well cell culture plates with the coatings orientated upward and then sterilized by UV radiation for 30 min. Each coverslip was soaked in ethanol for 2 h to remove any unreacted monomers, Cu²⁺ or residual solvents. The ethanol was then replaced by PBS and then EGM-2 medium. 20 μL medium which contained 5×10³ HUVECs cells was added dropwise onto the coverslips and the cells were allowed to attach for 3 hours before 1 mL of EGM-2 medium was added to each well of the cell culture plates. The cells were incubated at 37° C. with 5% CO₂. The medium exchange was carried out every 48 hours. A Vybrant® MTT Cell Proliferation Assay Kit (Invitrogen, CA) was used to measure the cell metabolic activity of the HUVECs after incubated for 1, 3 and 6 days. The absorbance was recorded using a SpectraMax M3 microplate reader. Live/dead assay was performed after cells incubated for 6 days using a LIVE/DEAD® Viability/Cytotoxicity Kit (Invitrogen, CA). The fluorescence microscopy images were recorded using a Nikon ECLIPSE Ti fluorescence microscope (Nikon Instruments Inc., NY). At day 6, phase-contrast images were also taken for both the Cu-PAS and PLGA wells on a Zeiss Axiovert 200 microscope equipped with a Dage 240 digital camera. All experiments were performed in triplicate.

In vivo biocompatibility study. All work with live animals was approved by the Cornell University Institutional Animal Care and Use Committee. Ethylene oxide-sterilized 14-Fe-PAS and poly(caprolactone) (PCL, Mn=80,000 Da, Sigma-Aldrich, MO) foams (Φ6 mm×H1 mm) were implanted in 25 female BALB/cJ mice (Jackson Laboratory) with an average age of 8-9 weeks. Under deep isoflurane-02 general anesthesia, the samples were subcutaneously implanted in the back of the mice by blunt dissection. After implantation for 4, 14, 28, 56 and 84 days, the animals were sacrificed and tissue samples (˜15×15 mm) surrounding the implants were harvested with the intact implants.

Tissues were fixed in 4% paraformaldehyde for 1.5 hours, and then soaked in 30% sucrose for 48 hours and embedded in Shandon™ Cryomatrix™ embedding resin (Thermo Scientific™). Serial cross-sections at the center, quarter and edge of each implant (8 μm thick, longitudinal axial cut) were stained with hematoxylin and eosin (H & E) and Masson's trichrome staining (MTS) to examine host responses such as inflammation, collagen deposition or any adverse effects. The macrophages distribution in and around the implants was also detected by immunofluorescence (IF) staining of CD68, as a pan macrophage marker, to explore the activities of macrophages with implant degradation. All reagents for H & E and MTS staining were obtained from Electron Microscopy Sciences, PA. For IF staining, the cross-sections were fixed in 2% (w/v) paraformaldehyde for 5 minutes, then incubated in 0.2% (v/v) Triton X-100 permeabilization solution for 10 minutes and 5% (v/v) goat serum blocking solution for 1 hour. Then the cross-sections were incubated with the primary antibodies of rat monoclonal anti-CD68 (5 μg/mL, Invitrogen, CA) for 1 hour at 37° C., followed by incubation with the goat anti-rat Alexa Fluor® 647 (1:200 dilution, Abcam, UK) for 1 hour at 37° C. Nuclei were counterstained with DAPI (4′,6-Diamidino-2-Phenylindole, Dihydrochloride). All immunofluorescent imaging was performed on a Nikon Eclipse Ti2-E inverted microscope, and image analysis performed with NIS Elements software (Tokyo, Japan). H & E and MTS stained sections were assessed by a board-certified veterinary pathologist, blinded to the identity of the polymer implant. The semi-quantitative scoring metric for inflammation around the implant: 0 means<10 cells per 400× field, 1 means 10-40 cells per 400× field, 2 means 40-80 cells per 400× field, 3 means>80 cells per 400× field. The scoring metric for inflammation within the implant: 0 means<2 cells per 400× field, 1 means 2-10 cells per 400× field, 2 means 10-25 cells per 400× field, 3 means>25 cells per 400× field. In most cases, the scores were based on the slides sectioned at center regions of implants. Slides sectioned at the center, quarter and edge regions were all checked by the pathologist. When notably different than the central sections, scores of the quarter and edge sections were added in and averaged.

Statistical analysis. All data were reported as the mean±standard deviation (SD). One-way ANOVA statistical analysis was performed to evaluate the significance of the experimental data, followed by a Tukey's post hoc test for pairwise comparison. All statistical analyses were executed using Kyplot 2.0 beta 15; a difference was considered significant when the p-value was less than 0.05 and the data were indicated with (*) for p<0.05, (**) for p<0.01 and (***) for p<0.001, respectively.

### Example 2. Decomposition of 11202 by Chelation Crosslinked Elastomers

The following is an example of methods and chelation crosslinked polymers of the present disclosure.

Materials and methods. Porous M-PAS elastomer scaffolds were prepared according to Example 1. PAS polymer of 9% ligand density were crosslinked via formation of coordination bond with Fe³⁺ or Cu²⁺. Granulated samples were prepared using cryomill. To evaluate the effect of 9-M-PAS elastomer on the self-decomposition of H₂O₂, 2.08 mM H₂O₂ was reacted with 0.3 mg of porous 9-M-PAS elastomer scaffolds at 37° C., mixing with stirring for 4-, 8-, and 24-hours, followed by measurement of concentration. 2.08 mM H₂O₂ was used as a control to monitor changes in concentration due to self-decomposition. Each reaction at each duration contained 3 replicates. Concentrations of H₂O₂ was quantified using horseradish peroxidase (HRP)-catalyzed oxidation of dihydrophenolxazine derivatives (Amplex Red)². A 25× dilution to the H₂O₂ sample solution aliquoted from reactions was mixed with Amplex Red (160 μM) and HRP (0.41 U/mL) in clear bottom black 96-well plates for measurement. Colorimetric readings of oxidation product of Amplex Red, resorufin, was measured with SpectraMax® M3 microplate reader (Molecular Devices). The filter wavelength was set at 570 nm. A set of H₂O₂ standard from 0 to 0.2 mM was used to correlate the optical density measurements with concentration. All of the data are expressed as mean±standard deviation. Statistical analysis was performed using JMP 15 (SAS Institute Inc.). Significance was assessed using one-way ANOVA with Tukey-Kramer HSD multiple comparisons. Differences were considered statistically significant at p<0.05.

Results. Concentrations of H₂O₂ were significantly different across 4-, 8-, and 24-hours reaction durations. 9-Cu-PAS elastomer accelerated the decomposition of H₂O₂ more than 9-Fe-PAS elastomer. By 4 hours of reaction, the concentration of H₂O₂ reacted with 9-Cu-PAS elastomer was 0.94±0.05 mM while with 9-Fe-PAS elastomer was 1.57±0.08 mM. By 24 hours, the concentration of H₂O₂ reacted with 9-Cu-PAS elastomer decreased to 0.01±0.01 mM, while with 9-Fe-PAS elastomer decreased less as 1.42±0.04 mM. Concentrations of H₂O₂ reacted with 9-Cu-PAS elastomer were significantly lower than that reacted with 9-Fe-PAS elastomer at 4-, 8-, and 24-hours, indicated that 9-Cu-PAS elastomer was more effective in accelerating the decomposition of H₂O₂. H₂O₂ concentration of 9-Cu-PAS elastomer at 4-, 8-, and 24-hours were significantly different from that of control. For 9-Fe-PAS elastomer, the difference was only significant at 4-hours of reaction.

### Example 3. Vascular Grafts Fabricated from Chelation Crosslinked Elastomers

The following is an example of a use of chelation crosslinked polymers of the present disclosure.

Method of 11-Zn-PAS grafts preparation. An 11-Zn-PAS elastomer was prepared by a previously described method of the disclosure, where the metal was zinc (Zn) and the ligand density of the PAS polymer was 11%. 11-Zn-PAS vascular grafts were fabricated by extrusion method. Ground NaCl particulates (32-53 μm in size) were first homogeneously mixed into 11-Zn-PAS acetone solution (50%, w/v) with polymer:NaCl=1:3 (w/w). Then, the 11-Zn-PAS/NaCl mixture were transferred into a 1 mL syringe. The mixture was then manually extruded into a tubular shape with a stainless-steel shaft (1.2 mm in diameter) as a mandrel and a PTFE tube (1.98 mm in diameter and 15 mm in length) as an outer sheath. The PTFE outer sheath was then removed. NaCl particulates of different sizes (23-32 μm: 32-53 μm: 53-72 μm=1:1:1, w/w/w) were mixed and attached onto the surface of the 11-Zn-PAS-salt tubes to form a thin salt layer. The 11-Zn-PAS-salt tubes were vacuum dried overnight at room temperature and then heated at 30 mTorr and 150° C. for 8 hours and cooled to room m temperature. The NaCl particulates were removed by immersing the 11-Zn-PAS-salt tubes in deionized water for 48 hours with water replacement every 6 hours. The porous grafts were then freeze-dried. Prior to in vivo implantation, the grafts were sterilized with ethylene oxide (Andersen Products), treated with gas plasma (Harrick Plasma Generator) for 5 mins in room air, and soaked into 180 units/mL (in saline solution) heparin overnight.

Method of grafts implantation. All procedures were approved by the Institutional Animal Care and Use Committee (IACUC) at the Cornell University following NIH guidelines for the care and use of laboratory animals. Male CD® (Sprague Dawley) IGS rats (strain code: 001, body weight=300-325 g, Charles River Laboratories, Boston, MA) were used for vascular graft common carotid artery interposition.

Rats were anesthetized by isoflurane inhalation (3% for induction, then 1.5% for maintenance). An incision at the midline of the neck was made and the left side muscles were retracted to expose the left common carotid artery. Blood flow of the common carotid artery was blocked with double microvascular clamps (Fine Science Tools, USA). A vascular graft was end-to-end anastomosed to the common carotid artery with 10-0 polyamide monofilament sutures (AROSurgical, Newport Beach, CA) by interrupted stitches. After the anastomosis, the microvascular clamps were removed from the common carotid artery to recover the blood flow. The surgical incision was closed with 4-0 absorbable sutures (Ethicon). No anticoagulation or antiplatelet treatments were administrated pre- and post-operatively. Analgesic (Buprenex, 0.03 mg/kg) were given once before the surgery and every 8 h for 48 h after the surgery.

Method of grafts retrieval and histological analysis. 3, 5, and 7 days after the surgery, rats were anesthetized, and the implanted vascular grafts were exposed through a midline incision made on the neck and dissected out from surrounding tissues for analysis.

The explanted vascular grafts were rinsed with heparinized saline solution, fixed in 4% paraformaldehyde (PFA, Electron Microscopy Sciences, USA) at 4° C. for 1 h, and soaked in 30% sucrose solution at 4° C. for 48 h. The vascular grafts were then embedded vertically into the Shandon™ Cryomatrix™ embedding resin (Thermo Scientific™), snap-frozen at −80° C., and serially cryosectioned at 5 μm thickness. The sample cross-sections were stained with hematoxylin and eosin (H & E) to examine host responses. All reagents for H &E staining were obtained from Electron Microscopy Sciences, PA, USA. All histological images were captured with an inverted microscope (Eclipse Ti2, Nikon, Japan) in brightfield.

Results. All rats survived without malignancy, infection, or abscess at implantation sites. All 11-Zn-PAS grafts were patent. At day 3, cells infiltrated the entire grafts. On day 7, there was more thorough cell infiltration and there were no visual signs of degradation of the grafts. The grafts maintained their’ original shape with no dilation, indicative of mechanical integrity. The 11-Zn-PAS grafts showed good host integration. Tissue around the 11-Zn-PAS grafts showed mild adverse responses such as inflammation. Inflammatory cells appeared in the implants at day 3 because of a nonspecific inflammatory response to the implants. The gradient suggests a surface to interior migration of the cells, as expected. At day 7, there were more host cells both in the luminal and abluminal side of the graft.

This is a study of the early phase of host interaction with the graft. All three grafts are patent with no visible signs of thrombosis, necrosis, or other severe adverse events. No graft material degradation was apparent at this early phase.

Although the present disclosure has been described with respect to one or more particular examples, it will be understood that other examples of the present disclosure may be made without departing from the scope of the present disclosure.

1. A chelation crosslinked polyester comprising:
a polyester backbone comprising one or more ester group(s), wherein the polyester backbone comprises one or more chelation crosslinking group(s) within and/or pendant from the polyester backbone; and
one or more cation(s),
wherein at least a portion of the cation(s) is/are bonded to at least a portion of the chelation crosslinking group(s) via one or more chelation crosslinking bond(s), thereby crosslinking the polyester backbone.

2. The chelation crosslinked polyester of claim 1, wherein at least one of or all of the chelation crosslinking group(s) is/are pendant from the polyester backbone.

3. The chelation crosslinked polyester of claim 1, wherein 0.01 mol % to 50 mol % of the ester group(s) comprise a chelation crosslinking group.

4. The chelation crosslinked polyester of claim 1, wherein one or more or all of the ester group(s) is/are aliphatic ester group(s).

5. The chelation crosslinked polyester of claim 1, wherein the polyester backbone has a molecular weight (M_{w} and/or Mₙ) of 1,000 to 10,000,000 g/mol and/or a polydispersity index of 1 to 5.

6. The chelation crosslinked polyester of claim 1, wherein the polyester backbone comprises the following structure: 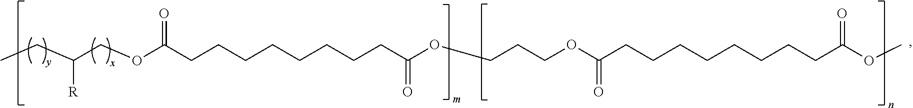
wherein R is a pendant chelation crosslinking group,
m is 0.01 to 100, and
n is 0 to 99.99.

7. The chelation crosslinked polyester of claim 1, wherein the chelation crosslinking group(s) is/are chosen from bidentate groups, tridentate groups, tetradendate groups, pentadentate groups, and combinations thereof.

8. The chelation crosslinked polyester of claim 1, wherein the chelation crosslinking group(s) is/are chosen from imine groups, carboxylate groups, aromatic heterocycle groups, amine groups, hydroxyl groups, ether groups, polyether groups, and crown ether groups, and combinations thereof.

9. The chelation crosslinked polyester of claim 1, wherein the chelation crosslinking group(s) is/are chosen from salicylaldimine groups, 2-vanillin groups, 2,3-dihydroxybenzaldehyde groups, 2,4-pyridinedicarbonyl dichloride groups, 2-[[3,4-bis[(triethylsilyl)oxy]phenyl]methyl]-oxirane groups, and [2,2′-Bipyridine]-5,5′-dicarbonyl dichloride groups, and combinations thereof.

10. The chelation crosslinked polyester of claim 1, wherein the cation(s) is/are chosen from Group(II) cations, transition metals, and combinations thereof.

11. The chelation crosslinked polyester of claim 1, wherein the cation(s) is/are present at 0.01% to 50% by weight, based on the total weight of the polyester and the cation(s).

12. The chelation crosslinked polyester of claim 1, wherein the mole ratio of the chelation crosslinking group(s) to the cation(s) is 1:1 to 6:1.

13. The chelation crosslinked polyester of claim 1, wherein at least a portion of the cation(s) is/are bonded to at least a portion of the chelation crosslinking group(s) via ionic bonds, coordinate covalent bonds, or a combination thereof.

14. The chelation crosslinked polyester of claim 1, wherein the crosslinking is reversible.

15. The chelation crosslinked polyester of claim 1, wherein the polyester backbone further comprises one or more functional group(s) enabling one or more inter- and/or intra-chain bond(s) other than the chelation crosslinking bond(s).

16. The chelation crosslinked polyester of claim 1, wherein the polyester backbone exhibits a glass transition temperature (T_{g}) below room temperature and is semicrystalline.

17. The chelation crosslinked polyester of claim 1, wherein the chelation crosslinked polyester exhibits one or more or all of the following:
biocompatibility;
biodegradability;
a porosity of 60% or greater;
a hysteresis of 100% or less;
a contact angle of 53° to 83°;
a strain of break of from 130% to 520%;
a Young's modulus of from 0.5 MPa to 4 MPa;
an ultimate tensile strength (UTS) of from 1485 kPa to 2300 kPa;
a water content of 0% to 50% by weight, based on the total weight of the polyester backbone and water.

18. The chelation crosslinked polyester of claim 1, wherein the chelation crosslinked polyester comprises a polyester copolymer backbone.

19. The chelation crosslinked polyester of claim 18, wherein the polyester copolymer backbone comprises one or more other block(s) chosen from one or more other hydrophilic block(s), one or more other hydrophobic block(s), and combinations thereof.

20. The chelation crosslinked polyester of claim 19, wherein the other hydrophilic block(s) is/are chosen from polyethylene glycol (PEG) blocks, polylactic acid (PLA) blocks, poly(acrylic acid) (PAA) blocks, and combinations thereof; and/or the other hydrophobic block(s) chosen from polyethylene terephthalate (PET) blocks, poly(caprolactone) (PCL) blocks, poly(methyl methacrylate) (PMMA) blocks, and combinations thereof.

21. A composition comprising one or more chelation crosslinked polyester(s) of claim 1.

22. The composition of claim 21, wherein, the composition is a biomedical composition,
a pharmaceutical composition, a chewing gum base, or a sealant.

23. The composition of claim 21, wherein the composition is a fiber, a film, a monolith, tube, or a foam.

24. A fiber comprising one or more chelation crosslinked polyester(s) of claim 1.

25. The fiber of claim 24, wherein the fiber comprises a blend of the chelation crosslinked polyester(s) with one or more other polymer(s) and/or one or more other polymeric material(s).

26. The fiber of claim 25, wherein the other polymer(s) and/or the other polymeric material(s) are chosen from polylactic acids (PLAs), polyglycolic acids (PGAs), PLGAs, poly(caprolactone)s (PCLs), polyethylene glycols (PEGs), polyethylene terephthalates (PETs), polypropylenes, polyethylenes, nylons, polystyrenes, poly(glycerol sebacate) (PGS), poly(methyl methacrylate) (PMMA), poly(acrylic acid) (PAA), and combinations thereof.

27. A material comprising a plurality of the fibers of claim 24, wherein the material is a fabric, and wherein, optionally, the fabric is a weave or braid of the fibers.

28. The material of claim 27, wherein the material further comprises one or more other fiber(s).

29. The material of claim 28, wherein the other fiber(s) are chosen from polylactic acid (PLA) fibers, (PCL) fibers, polyethylene glycol (PEG) fibers, PLGA, poly(lactide-co-caprolactone) (PLCL), poly(glycerol sebacate) (PGS), poly(methyl methacrylate) (PMMA), poly(acrylic acid) (PAA) fibers, and combinations thereof.

30. A tissue graft comprising one or more chelation crosslinked polyester(s) of claim 1.

31. The tissue graft of claim 30, wherein the tissue graft is a soft tissue graft.

32. The tissue graft of claim 31, wherein the soft tissue graft is a vascular graft.

33. The tissue graft of claim 32, wherein the vascular graft is an arterial graft.

34. The tissue graft of claim 33, wherein the arterial graft comprises a lumen diameter of 6 mm or less.

35. An article of manufacture comprising one or more chelation crosslinked polyester(s) of claim 1.

36. The article of manufacture of claim 35, wherein the article of manufacture is chosen from consumer goods, tires, gloves, gaskets, washers, toys, chewing gum, hoses, and balloons.

37. A chelation crosslinked polyester comprising:
a polyester backbone comprising one or more ester group(s), wherein the polyester backbone comprises one or more chelation crosslinking group(s) within and/or pendant from the polyester backbone and the polyester backbone comprises the following structure: 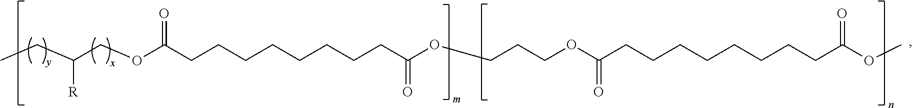
wherein R is a pendant chelation crosslinking group,
m is 0.01 to 100, and
n is 0 to 99.99; and
one or more cation(s),
wherein at least a portion of the cation(s) is/are bonded to at least a portion of the chelation crosslinking group(s) via one or more chelation crosslinking bond(s), thereby crosslinking the polyester backbone.

38. A chelation crosslinked polyester comprising:
a polyester backbone comprising one or more ester group(s), wherein the polyester backbone comprises one or more chelation crosslinking group(s) chosen from salicylaldimine groups, 2-vanillin groups, 2,3-dihydroxybenzaldehyde groups, 2,4-pyridinedicarbonyl dichloride groups, 2-[[3,4-bis[(triethylsilyl)oxy]phenyl]methyl]-oxirane groups, and [2,2′-Bipyridine]-5,5′-dicarbonyl dichloride groups, and combinations thereof within and/or pendant from the polyester backbone; and
one or more cation(s),
wherein at least a portion of the cation(s) is/are bonded to at least a portion of the chelation crosslinking group(s) via one or more chelation crosslinking bond(s), thereby crosslinking the polyester backbone.
